(19) United States
(12) Patent Application Publication
(10) Pub. No.: US 2018/0071303 A1
(43) Pub. Date: Mar. 15, 2018

(54) SYK INHIBITORS

(71) Applicant: Gilead Sciences, Inc., Foster City, CA (US)

(72) Inventors: Esteban M. Abella, Emerald Hills, CA (US); Arati V. Rao, Palo Alto, CA (US); Antonio Mario Querido Marcondes, Millbrae, CA (US)

(21) Appl. No.: 15/705,159

(22) Filed: Sep. 14, 2017

Related U.S. Application Data
(60) Provisional application No. 62/394,573, filed on Sep. 14, 2016.

(51) Int. Cl. A61K 31/5377; A61K 31/497

(52) U.S. Cl. CPC A61K 31/5377; A61K 2300/00; A61K 31/497

(57) ABSTRACT

The application provides methods reducing the side effects of chemotherapy and radiotherapy, including, hematopoietic toxicity, anemia, myelosuppression, pancytopenia, thrombocytopenia, neutropenia, lymphopenia, leukopenia, stomatitis and alopecia. The application provides a method for increasing the number of, neutrophil counts and platelet counts in a patient in need thereof, comprising administering an effective of an inhibitor of spleen tyrosine kinase (SYKi). The present application provides methods for treating myelosuppresive disorders by the administration of a SYKi. In certain embodiments, the myelosuppression is induced by the administration of one or more myelosuppressive agents, for example, anti-cancer drugs. The present application provides methods for treating AML/ALL in a patient with 11q23/MLL abnormalities comprising the step of administering an effective amount of a SYKi to said patient.

### CROSS-REFERENCE TO RELATED APPLICATION

This application claims the benefit under 35 U.S.C. § 119(e) of U.S. Provisional Application No. 62/394,573, filed Sep. 14, 2016, which is hereby incorporated by reference in its entirety.

### FIELD

The present disclosure relates to methods of treatment of diseases and disorders using compounds and compositions that inhibit Spleen Tyrosine Kinase (SYK) activity.

### BACKGROUND

Protein kinases, the largest family of human enzymes, encompass well over 500 proteins. Spleen Tyrosine Kinase (SYK) is a member of the family of tyrosine kinases, and is a regulator of early B-cell development as well as mature B-cell activation, signaling, and survival. SYK has roles in immunoreceptor- and integrin-mediated signaling in a variety of cell types, including B-cells, macrophages, monocytes, mast cells, eosinophils, basophils, neutrophils, dendritic cells, T-cells, natural killer cells, platelets, and osteoclasts. The inhibition of Syk activity can be useful for the treatment cancers and inflammatory diseases. U.S. Pat. Nos. 8,455,493, 8,440,667, 9,376,441, 9,416,111, 9,353,066 and 9,376,418 disclose SYK inhibitors. Several SYK inhibitors are in advanced stages of clinical trials. Fostamatinib is a SYK inhibitor currently undergoing phase III clinical trials for chronic immune thrombocytopenic purpura (chronic ITP). (NCT02076412; Clinicaltrials.gov). Fostamatinib, however, have adverse side effects including hypertension, neutropenia and transaminitis. (Nijjar J. S. et. al., Rheumatology 2013, 1556-1562).

Cytotoxic chemotherapy drugs can produce side effects including lowering the level of cells in the bone marrow, resulting in abnormally low number of cells in the blood, a condition called myelosuppression. The effects of myelosuppression include anemia (low red blood cell counts), neutropenia (low neutrophils counts), leucopenia (low white blood cell counts), and thrombocytopenia (low platelet counts). (http://www.biomodels. com/animal-models/cancer-supportive-care/myelosuppression; Carey P J, Drug Safety, 2003: 691-706.). Among the drugs causing significant myelosuppression effects are cytotoxic antibiotics and antibiotic derivatives, other cytotoxic drugs, antimetabolites, alkaloid-type anti-tumor agents, alkylating agents, and heavy metal complexes (for example, platinum complexes). A treatment for the side effects of these drugs would be a highly welcome addition to the field of cancer treatment. (U.S. Pat. Nos. 5,035,878, 7,338,938). In addition, there is a need to advance therapies that can reduce the side effects or improve the efficacy associated with chemotherapy and radiotherapy.

### SUMMARY

The application provides methods reducing the side effects of chemotherapy and radiotherapy, including, hematopoietic toxicity, anemia, myelosuppression, pancytopenia, thrombocytopenia, neutropenia, lymphopenia, leukopenia, stomatitis and alopecia. The application provides methods for increasing the number of, neutrophil counts and platelet counts in a patient in need thereof and reducing the leukemic burden or tumor burden, comprising administering an effective amount of an inhibitor of spleen tyrosine kinase (SYKi). The present application provides methods for treating myelosuppresive disorders by the administration of an inhibitor of spleen tyrosine kinase (SYKi). In certain embodiments, myelosuppression is induced by the administration of one or more myelosuppressive agents, for example, anti-cancer drugs.

The present application provides methods for increasing bone marrow production, neutrophil count or platelet count in a patient diagnosed with myelodysplastic syndrome (MDS) or preleukemia, or acute leukemia including AML and ALL comprising the step of administering an effective amount of an inhibitor of spleen tyrosine kinase (SYKi). In certain embodiments, the SYKi is administered to a patient receiving bone marrow transplant.

The present application provides methods for treating leukemia, including AML and ALL, in a patient with 11q23/MLL abnormalities, comprising the step of administering an effective amount of an inhibitor of spleen tyrosine kinase (SYKi) to said patient.

### DESCRIPTION OF THE FIGURES

FIG. 1 shows neutrophil counts in patients receiving chemotherapy and 200 or 400 mg twice daily of entospletinib (also referred to herein as Compound 1, ENTO or GS-9973). The figure shows daily levels for individual patients.

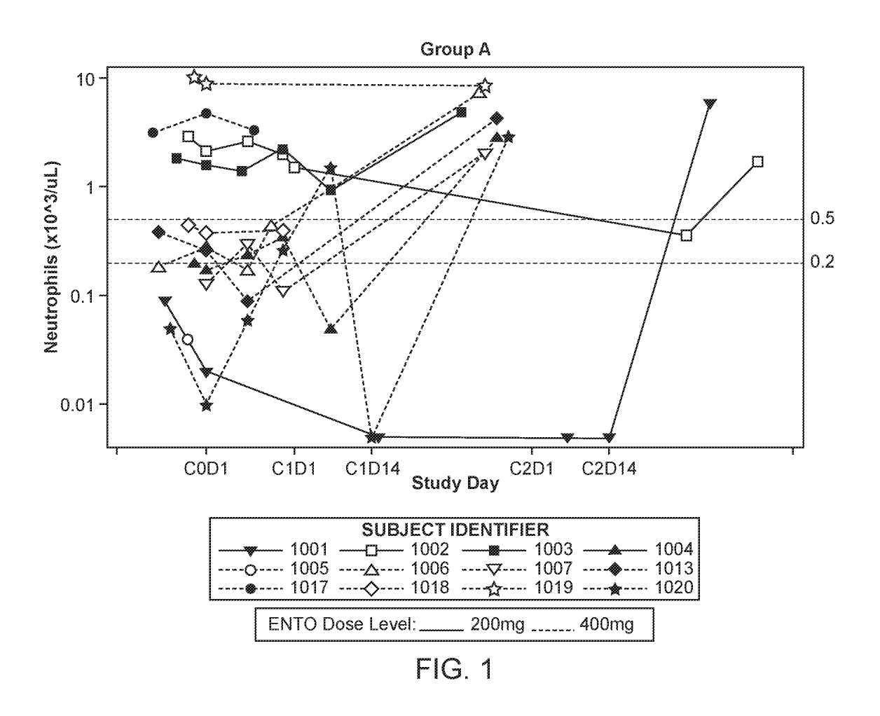

FIG. 2 shows platelet counts in patients receiving chemotherapy and 200 mg or 400 mg doses of entospletinib (also referred to herein as Compound 1, ENTO or GS-9973). The figure shows daily levels for individual patients.

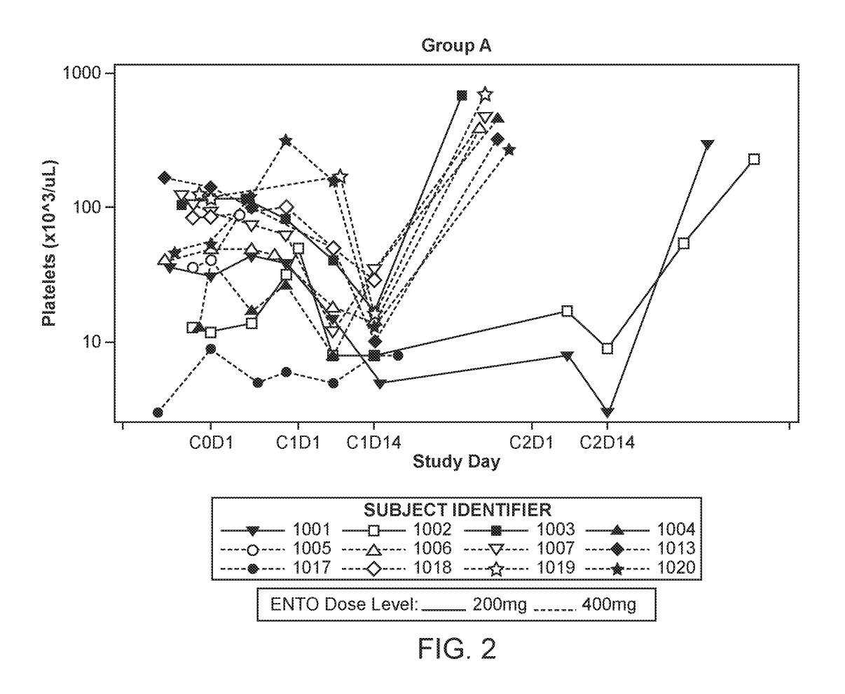

FIG. 3 shows platelet counts in patients receiving chemotherapy and 200 mg or 400 mg doses of entospletinib (also referred to herein as Compound 1, ENTO or GS-9973). The figure shows levels for individual patients.

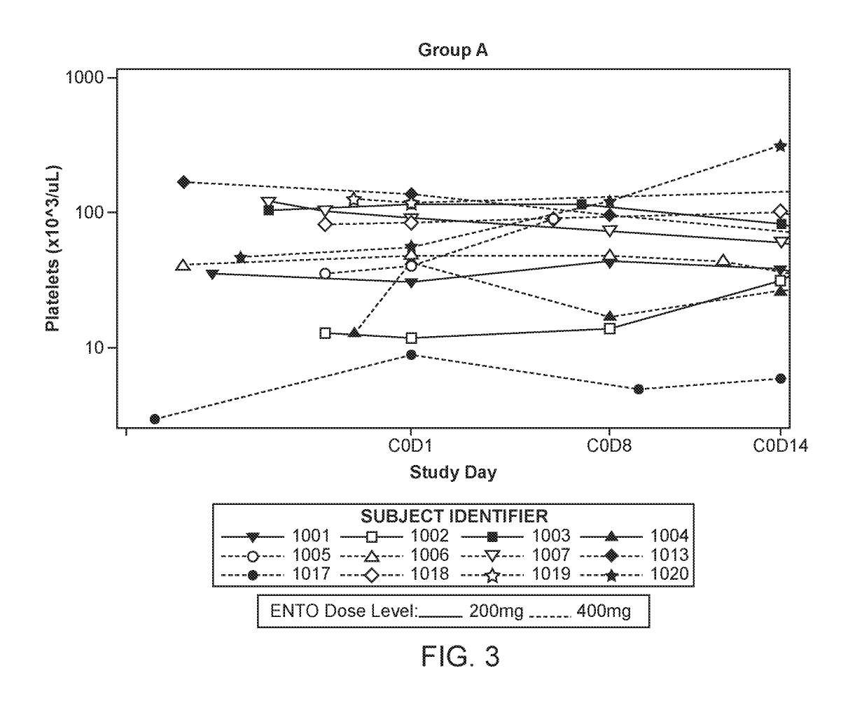

FIG. 4 shows circulating blast levels in patients receiving chemotherapy and 200 or 400 mg doses of entospletinib (also referred to herein as Compound 1, ENTO or GS-9973). The figure shows daily levels for individual patients.

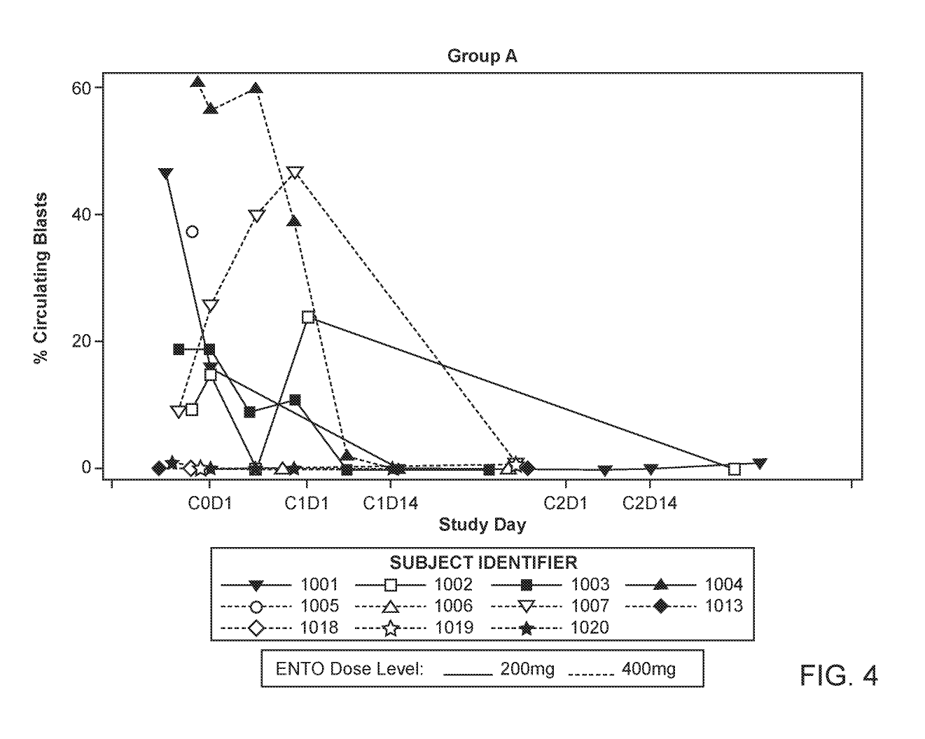

FIG. 5 shows comparison of inhibitory activity of entospletinib (also referred to herein as Compound 1, ENTO or GS-9973) and R406 against a series of kinases. (Adapted from Currie et. al., J. Med. Chem., 2014, 57 (9), 3856-3873).

### DETAILED DESCRIPTION

As used herein, when any variable occurs more than one time in a chemical formula, its definition on each occurrence is independent of its definition at every other occurrence. In accordance with the usual meaning of “a” and “the” in patents, reference, for example, to “a” kinase or “the” kinase is inclusive of one or more kinases.

As used in the present specification, the following words, phrases and symbols are generally intended to have the meanings as set forth below, except to the extent that the context in which they are used indicates otherwise. The following abbreviations and terms have the indicated meanings throughout:

A dash (“-”) that is not between two letters or symbols is used to indicate a point of attachment for a substituent. For example, —CONH₂ is attached through the carbon atom.

By “optional” or “optionally” is meant that the subsequently described event or circumstance may or may not occur, and that the description includes instances where the event or circumstance occurs and instances in which it does not. For example, “optionally substituted alkyl” encompasses both “alkyl” and “substituted alkyl” as defined below. It will be understood by those skilled in the art, with respect to any group containing one or more substituents, that such groups are not intended to introduce any substitution or substitution patterns that are sterically impractical, synthetically non-feasible and/or inherently unstable.

“Alkyl” encompasses straight chain and branched chain having the indicated number of carbon atoms, usually from 1 to 20 carbon atoms, for example 1 to 8 carbon atoms, such as 1 to 6 carbon atoms. For example C₁-C₆ alkyl encompasses both straight and branched chain alkyl of from 1 to 6 carbon atoms. Examples of alkyl groups include methyl, ethyl, propyl, isopropyl, n-butyl, sec-butyl, tert-butyl, pentyl, 2-pentyl, isopentyl, neopentyl, hexyl, 2-hexyl, 3-hexyl, 3-methylpentyl, and the like. Alkylene is another subset of alkyl, referring to the same residues as alkyl, but having two points of attachment. Alkylene groups will usually have from 2 to 20 carbon atoms, for example 2 to 8 carbon atoms, such as from 2 to 6 carbon atoms. For example, C₀ alkylene indicates a covalent bond and C₁ alkylene is a methylene group. When an alkyl residue having a specific number of carbons is named, all geometric isomers having that number of carbons are intended to be encompassed; thus, for example, “butyl” is meant to include n-butyl, sec-butyl, isobutyl and t-butyl; “propyl” includes n-propyl and isopropyl. “Lower alkyl” refers to alkyl groups having 1 to 4 carbons.

“Alkenyl” indicates an unsaturated branched or straight-chain alkyl group having at least one carbon-carbon double bond derived by the removal of one molecule of hydrogen from adjacent carbon atoms of the parent alkyl. The group may be in either the cis or trans configuration about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl; and the like. In some embodiments, an alkenyl group has from 2 to 20 carbon atoms and in other embodiments, from 2 to 6 carbon atoms.

“Cycloalkyl” indicates a saturated hydrocarbon ring group, having the specified number of carbon atoms, usually from 3 to 7 ring carbon atoms. Examples of cycloalkyl groups include cyclopropyl, cyclobutyl, cyclopentyl, and cyclohexyl as well as bridged and caged saturated ring groups such as norbornane.

By “alkoxy” is meant an alkyl group of the indicated number of carbon atoms attached through an oxygen bridge such as, for example, methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, 3-methylpentoxy, and the like. Alkoxy groups will usually have from 1 to 6 carbon atoms attached through the oxygen bridge. “Lower alkoxy” refers to alkoxy groups having 1 to 4 carbons.

“Aminocarbonyl” encompasses a group of the formula —(C═O)NRₐR_{b} where Rₐ and R_{b} are independently chosen from hydrogen and the optional substituents for “substituted amino” described below.

“Acyl” refers to the groups (alkyl)-C(O)—; (cycloalkyl)-C(O)—; (aryl)-C(O)—; (heteroaryl)-C(O)—; and (heterocycloalkyl)-C(O)—, wherein the group is attached to the parent structure through the carbonyl functionality and wherein alkyl, cycloalkyl, aryl, heteroaryl, and heterocycloalkyl are as described herein. Acyl groups have the indicated number of carbon atoms, with the carbon of the keto group being included in the numbered carbon atoms. For example a C₂ acyl group is an acetyl group having the formula CH₃(C═O)—.

By “alkoxycarbonyl” is meant an ester group of the formula (alkoxy)(C═O)-attached through the carbonyl carbon wherein the alkoxy group has the indicated number of carbon atoms. Thus a C₁-C₆alkoxycarbonyl group is an alkoxy group having from 1 to 6 carbon atoms attached through its oxygen to a carbonyl linker.

By “amino” is meant the group —NH₂.

“Aryl” encompasses 5- and 6-membered carbocyclic aromatic rings, for example, benzene: bicyclic ring systems wherein at least one ring is carbocyclic and aromatic, for example, naphthalene, indane, and tetralin; and tricyclic ring systems wherein at least one ring is carbocyclic and aromatic, for example, fluorene. For example, aryl includes 5- and 6-membered carbocyclic aromatic rings fused to a 5- to 7-membered heterocycloalkyl ring containing 1 or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap in any way with heteroaryl, separately defined below. Hence, if one or more carbocyclic aromatic rings is fused with a heterocycloalkyl aromatic ring, the resulting ring system is heteroaryl, not aryl, as defined herein.

The term “aryloxy” refers to the group —O-aryl.

The term “halo” includes fluoro, chloro, bromo, and iodo, and the term “halogen” includes fluorine, chlorine, bromine, and iodine.

“Heteroaryl” encompasses 5- to 7-membered aromatic, monocyclic rings containing one or more, for example, from 1 to 4, or In some embodiments, from 1 to 3, heteroatoms chosen from N, O, and S, with the remaining ring atoms being carbon; and bicyclic heterocycloalkyl rings containing one or more, for example, from 1 to 4, or In some embodiments, from 1 to 3, heteroatoms chosen from N, O, and S, with the remaining ring atoms being carbon and wherein at least one heteroatom is present in an aromatic ring. For example, heteroaryl includes a 5- to 7-membered heterocycloalkyl, aromatic ring fused to a 5- to 7-membered cycloalkyl ring. For such fused, bicyclic heteroaryl ring systems wherein only one of the rings contains one or more heteroatoms, the point of attachment may be at the heteroaromatic ring or the cycloalkyl ring. When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. Examples of heteroaryl groups include, but are not limited to, (as numbered from the linkage position assigned priority 1), 2-pyridyl, 3-pyridyl, 4-pyridyl, 2,3-pyrazinyl, 3,4-pyrazinyl, 2,4-pyrimidinyl, 3,5-pyrimidinyl, 2,3-pyrazolinyl, 2,4-imidazolinyl, isoxazolinyl, oxazolinyl, thiazolinyl, thiadiazolinyl, tetrazolyl, thienyl, benzothiophenyl, furanyl, benzofuranyl, benzimidazolinyl, indolinyl, pyridizinyl, triazolyl, quinolinyl, pyrazolyl, and 5,6,7,8-tetrahydroisoquinoline. Bivalent radicals derived from univalent heteroaryl radicals whose names end in “-yl” by removal of one hydrogen atom from the atom with the free valence are named by adding “-idene” to the name of the corresponding univalent radical, e.g., a pyridyl group with two points of attachment is a pyridylidene. Heteroaryl does not encompass or overlap with aryl as defined above. Substituted heteroaryl also includes ring systems substituted with one or more oxide (—O⁻) substituents, such as pyridinyl N-oxides.

The term “heteroaryloxy” refers to the group —O-heteroaryl.

By “heterocycloalkyl” is meant a single aliphatic ring, usually with 3 to 7 ring atoms, containing at least 2 carbon atoms in addition to 1-3 heteroatoms independently selected from oxygen, sulfur, and nitrogen, as well as combinations comprising at least one of the foregoing heteroatoms. Suitable heterocycloalkyl groups include, for example (as numbered from the linkage position assigned priority 1), 2-pyrrolinyl, 2,4-imidazolidinyl, 2,3-pyrazolidinyl, 2-piperidyl, 3-piperidyl, 4-piperidyl, and 2,5-piperazinyl. Morpholinyl groups are also contemplated, including 2-morpholinyl and 3-morpholinyl (numbered wherein the oxygen is assigned priority 1). Substituted heterocycloalkyl also includes ring systems substituted with one or more oxo moieties, such as piperidinyl N-oxide, morpholinyl-N-oxide, 1-oxo-1-thiomorpholinyl and 1,1-dioxo-1-thiomorpholinyl.

“Heterocycloalkyl” also includes bicyclic ring systems wherein neither of the rings is aromatic and wherein at least one of the rings in the bicyclic ring system contains at least 2 carbon atoms in addition to 1-3 heteroatoms independently chosen from oxygen, sulfur, and nitrogen.

The term “heterocycloalkyloxy” refers to the group —O-heterocycloalkyl.

The term “nitro” refers to the group —NO₂.

The term “phosphono” refers to the group —PO₃H₂.

“Thiocarbonyl” refers to the group —C(═O)SH.

The term “optionally substituted thiocarbonyl” includes the groups —C(═O)S-(optionally substituted (C₁-C₆)alkyl), —C(═O)S-(optionally substituted aryl), —C(═O)S-(optionally substituted heteroaryl), and —C(═O)S-(optionally substituted heterocycloalkyl).

The term “sulfanyl” includes the groups —S-(optionally substituted (C₁-C₆)alkyl), —S-(optionally substituted aryl), —S-(optionally substituted heteroaryl), and —S-(optionally substituted heterocycloalkyl). Hence, sulfanyl includes the group C₁-C₆ alkylsulfanyl.

The term “sulfinyl” includes the groups —S(O)H, —S(O)-(optionally substituted (C₁-C₆)alkyl), —S(O)-optionally substituted aryl), —S(O)-optionally substituted heteroaryl), —S(O)-(optionally substituted heterocycloalkyl), and —S(O)-(optionally substituted amino).

The term “sulfonyl” includes the groups —S(O₂) H, —S(O₂)-(optionally substituted (C₁-C₆)alkyl), —S(O₂)-optionally substituted aryl), —S(O₂)-optionally substituted heteroaryl), —S(O₂)-(optionally substituted heterocycloalkyl), —S(O₂)-(optionally substituted alkoxy), —S(O₂)-optionally substituted aryloxy), —S(O₂)-optionally substituted heteroaryloxy), —S(O₂)-(optionally substituted heterocyclyloxy), and —S(O₂)-(optionally substituted amino).

The term “substituted,” as used herein, means that any one or more hydrogens on the designated atom or group is replaced with a selection from the indicated group, provided that the designated atom's normal valence is not exceeded. When a substituent is oxo (i.e., ═O) then 2 hydrogens on the atom are replaced. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation as an agent having at least practical utility. Unless otherwise specified, substituents are named into the core structure. For example, it is to be understood that when (cycloalkyl)alkyl is listed as a possible substituent, the point of attachment of this substituent to the core structure is in the alkyl portion.

The terms “substituted” alkyl, cycloalkyl, aryl, heterocycloalkyl, and heteroaryl (including without limitation dihydrobenzoxazinyl, dihydroquinoxalinyl, dihydrobenzodiazolyl, dihydroindolyl, pyrimidinyl, quinolinyl, indazolyl, indolyl, benzoimidazolyl, benzothioxolyl, benzotriazolyl, quinoxalinyl, quinazolinyl, morpholinyl, azetidinyl, pyrrolidinyl, oxanyl, pyridinyl, oxazolyl, piperazinyl, and pyridazinyl group), unless otherwise expressly defined, refer respectively to alkyl, cycloalkyl, aryl, heterocycloalkyl, and heteroaryl (including without limitation dihydrobenzoxazinyl, dihydroquinoxalinyl, dihydrobenzodiazolyl, dihydroindolyl, pyrimidinyl, quinolinyl, indazolyl, indolyl, benzoimidazoyl, benzothioxolyl, benzotriazolyl, quinoxalinyl, quinazolinyl, morpholinyl, azetidinyl, pyrrolidinyl, oxanyl, pyridinyl, oxazolyl, piperazinyl, and pyridazinyl group) wherein one or more (such as up to 5, for example, up to 3) hydrogen atoms are replaced by a substituent independently chosen from —R^{a}, —OR^{b}, —O(C₁-C₂ alkyl)O— (e.g., methylenedioxy-), —SR^{b}, guanidine, guanidine wherein one or more of the guanidine hydrogens are replaced with a lower-alkyl group, —NR^{b}R^{c}, halo, cyano, oxo (as a substituent for heterocycloalkyl), nitro, —COR^{b}, —CO₂R^{b}, —CONR^{b}R^{c}, —OCOR^{b}, —OCO₂R^{a}, —OCONR^{b}R^{c}, —NR^{c}COR^{b}, —NR^{c}CO₂R^{a}, —NR^{c}CONR^{b}R^{c}, —SOR^{a}, —SO₂R^{a}, —SO₂NR^{b}R^{c}, and —NR^{c}SO₂R^{a},

where R^{a} is chosen from optionally substituted C₁-C₆ alkyl, optionally substituted cycloalkyl, optionally substituted aryl, optionally substituted heterocycloalkyl, and optionally substituted heteroaryl;

R^{b} is chosen from H, optionally substituted C₁-C₆ alkyl, optionally substituted aryl, and optionally substituted heteroaryl; and

R^{c} is chosen from hydrogen and optionally substituted C₁-C₄ alkyl; or

R^{b} and R^{c}, and the nitrogen to which they are attached, form an optionally substituted heterocycloalkyl group; and

where each optionally substituted group is unsubstituted or independently substituted with one or more, such as one, two, or three, substituents independently selected from C₁-C₄ alkyl, C₃-C₆ cycloalkyl, aryl, heteroaryl, aryl-C₁-C₄ alkyl-, heteroaryl-C₁-C₄ alkyl-, C₁-C₄ haloalkyl-, —OC₁-C₄ alkyl, —OC₁-C₄ alkylphenyl, —C₁-C₄ alkyl-OH, —C₁-C₄ alkyl-O—C₁-C₄ alkyl, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄ haloalkyl).

The term “substituted acyl” refers to the groups (substituted alkyl)-C(O)—; (substituted cycloalkyl)-C(O)—, (substituted aryl)-C(O)—, (substituted heteroaryl)-C(O)—, and (substituted heterocycloalkyl)-C(O)—, wherein the group is attached to the parent structure through the carbonyl functionality and wherein substituted alkyl, cycloalkyl, aryl, heteroaryl, and heterocycloalkyl are as described herein.

The term “substituted alkoxy” refers to alkoxy wherein the alkyl constituent is substituted (i.e., —O-(substituted alkyl)) wherein “substituted alkyl” is as described herein.

The term “substituted alkoxy carbonyl” refers to the group (substituted alkyl)-O—C(O)— wherein the group is attached to the parent structure through the carbonyl functionality and wherein “substituted alkyl” is as described herein.

The term “substituted aryloxy” refers to aryloxy wherein the aryl constituent is substituted (i.e., —O-(substituted aryl)) wherein “substituted aryl” is as described herein.

The term “substituted heteroaryloxy” refers to heteroaryloxy wherein the aryl constituent is substituted (i.e., —O-(substituted heteroaryl)) wherein “substituted heteroaryl” is as described herein.

The term “substituted cycloalkyloxy” refers to cycloalkyloxy wherein the cycloalkyl constituent is substituted (i.e., —O-(substituted cycloalkyl)) wherein “substituted cycloalkyl” is as described herein.

The term “substituted heterocycloalkyloxy” refers to heterocycloalkyloxy wherein the alkyl constituent is substituted (i.e., —O-(substituted heterocycloalkyl)) wherein “substituted heterocycloalkyl” is as described herein.

The term “substituted amino” refers to the group —NH—R^{d} or —NR^{d}R^{d} where each R^{d} is independently chosen from: hydroxy, optionally substituted alkyl, optionally substituted cycloalkyl, optionally substituted acyl, aminocarbonyl, optionally substituted aryl, optionally substituted heteroaryl, optionally substituted heterocycloalkyl, alkoxycarbonyl, sulfinyl and sulfonyl, provided that only one R^{d} may be hydroxyl, and wherein substituted alkyl, cycloalkyl, aryl, heterocycloalkyl, and heteroaryl refer respectively to alkyl, cycloalkyl, aryl, heterocycloalkyl, and heteroaryl wherein one or more (such as up to 5, for example, up to 3) hydrogen atoms are replaced by a substituent independently selected from —R^{a}, —OR^{b}, —O(C₁-C₂ alkyl)O— (e.g., methylenedioxy-), —SR^{b}, guanidine, guanidine wherein one or more of the guanidine hydrogens are replaced with a lower-alkyl group, —NR^{b}R^{c}, halo, cyano, nitro, —COR^{b}, —CO₂R^{b}, —CONR^{b}R^{c}, —OCOR^{b}, —OCO₂R^{a}, —OCONR^{b}R^{c}, —NR^{c}COR^{b}, —NR^{c}CO₂R^{a}, —NR^{c}CONR^{b}R^{c}, —SOR^{a}, —SO₂R^{a}, —SO₂NR^{b}R^{c} and —NR^{c}SO₂R^{a},

where R^{a} is optionally substituted C₁-C₆ alkyl, optionally substituted aryl, or optionally substituted heteroaryl;

R^{b} is hydrogen, optionally substituted C₁-C₆ alkyl, optionally substituted aryl, or optionally substituted heteroaryl; and

R^{c} is hydrogen and optionally substituted C₁-C₄ alkyl; or

R^{b} and R^{c}, and the nitrogen to which they are attached, form an optionally substituted heterocycloalkyl group; and

where each optionally substituted group is unsubstituted or independently substituted with one or more, such as one, two, or three, substituents independently selected from C₁-C₄ alkyl, aryl, heteroaryl, aryl-C₁-C₄ alkyl-, heteroaryl-C₁-C₄ alkyl-, C₁-C₄ haloalkyl-, —OC₁-C₄ alkyl, —OC₁-C₄ alkylphenyl, —C₁-C₄ alkyl-OH, —OC₁-C₄ haloalkyl, halo, —OH, —NH₂, —C₁-C₄ alkyl-NH₂, —N(C₁-C₄ alkyl)(C₁-C₄ alkyl), —NH(C₁-C₄ alkyl), —N(C₁-C₄ alkyl)(C₁-C₄ alkylphenyl), —NH(C₁-C₄ alkylphenyl), cyano, nitro, oxo (as a substitutent for heteroaryl), —CO₂H, —C(O)OC₁-C₄ alkyl, —CON(C₁-C₄ alkyl)(C₁-C₄ alkyl), —CONH(C₁-C₄ alkyl), —CONH₂, —NHC(O)(C₁-C₄ alkyl), —NHC(O)(phenyl), —N(C₁-C₄ alkyl)C(O)(C₁-C₄ alkyl), —N(C₁-C₄ alkyl)C(O)(phenyl), —C(O)C₁-C₄ alkyl, —C(O)C₁-C₄ phenyl, —C(O)C₁-C₄ haloalkyl, —OC(O)C₁-C₄ alkyl, —SO₂(C₁-C₄ alkyl), —SO₂(phenyl), —SO₂(C₁-C₄ haloalkyl), —SO₂NH₂, —SO₂NH(C₁-C₄ alkyl), —SO₂NH(phenyl), —NHSO₂(C₁-C₄ alkyl), —NHSO₂(phenyl), and —NHSO₂(C₁-C₄haloalkyl); and

wherein optionally substituted acyl, aminocarbonyl, alkoxycarbonyl, sulfinyl and sulfonyl are as defined herein.

The term “substituted amino” also refers to N-oxides of the groups —NHR^{d}, and NR^{d}R^{d} each as described above. N-oxides can be prepared by treatment of the corresponding amino group with, for example, hydrogen peroxide or m-chloroperoxybenzoic acid. The person skilled in the art is familiar with reaction conditions for carrying out the N-oxidation.

Provided herein are methods reducing the side effects of chemotherapy and radiotherapy, including, hematopoietic toxicity, anemia, myelosuppression, pancytopenia, thrombocytopenia, neutropenia, lymphopenia, leukopenia, stomatitis and alopecia, comprising the step of administering an effective amount of an inhibitor of spleen tyrosine kinase (SYKi) to a patient in need thereof. The application provides a method for increasing the neutrophil counts and platelet counts in a patient in need thereof, comprising administering an effective of an inhibitor of spleen tyrosine kinase (SYKi).

Also provided are methods for treating and alleviating myelosuppresive disorders by the administration of an inhibitor of spleen tyrosine kinase (SYKi). In certain embodiments, myelosuppression is induced by the administration of one or more myelosuppressive agents, for example, anti-cancer drugs. Myelosuppression includes neutropenia, pancytopenia, thrombocytopenia, leukopenia and anemia. In certain embodiments, the methods are provided for treating a patient suffering from hemolytic anemia, aplastic anemia or pure red cell anemia comprising the step of administering an effective amount of a SYKi. The application further provides methods for reducing the production of excessive reactive oxygen species in patients suffering from proliferative diseases, including cancer, and in particular, hematopoietic malignancies. In certain embodiments, methods are provided for protecting and stimulating proliferation of transplanted cells in the bone marrow of a human recipient of a bone marrow transplant, wherein prior to the bone marrow transplant, the recipient has received a myelosuppressing amount of an anti-cancer drug, said process comprising administering to the recipient an effective amount of a SYKi.

In certain embodiments, the application provides methods for treating chemotherapy or radiotherapy induced myelosuppression by the administration of a SYKi. In certain embodiments, the application provides a method of increasing the platelet count in a patient receiving chemotherapy or radiotherapy comprising the step of administering an effective amount of a SYKi. In certain embodiments, the application provides a method of increasing the neutrophil count in a patient receiving chemotherapy or radiotherapy by administering an effective amount of a SYKi.

In certain embodiments, the SYKi is administered as pre-treatment to chemotherapy or radiotherapy, wherein a SYKi is administered one day, two days, three days, four days, five days, six days, one week, two weeks, three weeks, one month or more than one month prior to the initiation of chemotherapy or radiotherapy.

In certain embodiments, the application provides methods of treatment for myelosuppression induced by chemotherapy and radiotherapy, and enhance the effectiveness of chemotherapy and decrease leukemic burden in patients undergoing chemotherapy. For example, chemotherapy or radiotherapy can reduce the neutrophil or platelet counts in a patient by 10%, 20%, 30%, 40%, 50%, 60%, 70%, 80% or 90% following the initiation of chemotherapy or radiotherapy. Provided herein are methods wherein the neutrophil or platelet count of a patient is increased following the completion of chemotherapy. For example, the neutrophil or platelet count can be returned to within 10% or 20% of the counts prior to the initiation of chemotherapy or radiotherapy comprising the administration of an effective amount of a SYKi, in particular, Compound 1 (also referred to as entospletinib, ENTO or GS-9973):

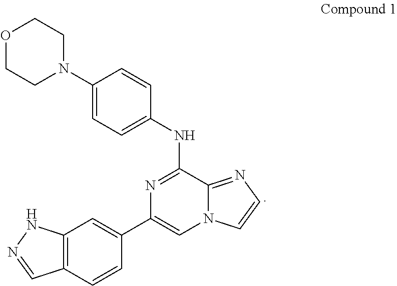 [SMILES: c1cn2cc(-c3ccc4cn[nH]c4c3)nc(Nc3ccc(N4CCOCC4)cc3)c2n1]

An inhibitor of spleen tyrosine kinase (SYKi) can be administered while the patient is undergoing treatment with one or more chemotherapy agents, such as DNA damaging agents, antibiotic agents, antimitotic agents, steroids glucocorticoids and combinations thereof. DNA alkylating agents can be selected from, for example, actinomycin, amsacrine, busulfan, carboplatin, chlorambucil, cisplatin, cyclophosphamide, Cytoxan, dactinomycin, daunorubicin, doxorubicin, epirubicin, iphosphamide, melphalan, merchlorehtamine, mitomycin, mitoxantrone, nitrosourea, procarbazine, taxol, taxotere, teniposide, etoposide and triethylenethiophosphoramide. The antibiotic chemotherapy agents can be selected from dactinomycin (actinomycin D), daunorubicin, doxorubicin (adriamycin), idarubicin, anthracyclines, mitoxantrone, bleomycins, plicamycin (mithramycin) and mitomycin. The antimitotic agents can be selected from vinca alkaloids and taxanes, nocodazole, epothilones, navelbine and epidipodophyllotoxin. Vinca alkaloids can be selected from vinblastine and vincristine, or derivatives thereof. Taxanes can be selected from paclitaxel and docetaxel, or derivatives thereof. In addition, the SYKi can be administered to a patient in combination with a second agent, for example, enasidenib, a dinaciclib compound (U.S. Patent Publication No. 2016/0193334), MK-3475, volasterib, midostaurin, gilteritinb, quizartinib, guadecitabine, sapacitabine, vosaroxin, vyxeos, ozogamicin, talirlne or an IDH2 inhibitor.

In certain embodiments, the application provides methods of treatment for myelosupprssion in a patient diagnosed with or receiving treatment for acute lymphocytic leukemia (ALL), acute myeloid leukemia (AML), chronic lymphocytic leukemia (CLL), small lymphocytic lymphoma (SLL), myeloproliferative leukemia (MPL), myelodysplastic syndrome (MDS), myeloproliferative disease (MPD), chronic myeloid leukemia (CML), multiple myeloma (MM), non-Hodgkin's lymphoma (NHL), mantle cell lymphoma (MCL), follicular lymphoma, Waldestrom's macroglobulinemia (WM), T-cell lymphoma, B-cell lymphoma, diffuse large B-cell lymphoma (DLBCL), pancreatic cancer, bladder cancer, colorectal cancer, breast cancer, prostate cancer, renal cancer, hepatocellular cancer, lung cancer, ovarian cancer, cervical cancer, gastric cancer, esophageal cancer, head and neck cancer, melanoma, neuroendocrine cancer, CNS cancer, brain cancer, bone cancer, soft tissue sarcoma, non-small cell lung cancer, small-cell lung cancer and colon cancer. In certain embodiments, the patient is diagnosed with acute ALL, AML, CML, CLL, MPD, MM, NHL, MCL or DLBCL.

In certain embodiments, the application provides methods of treatment for myelosupprssion in a patient diagnosed with or receiving treatment for solid tumor including but is not limited to prostate cancer, pancreatic cancer, bladder cancer, colorectal cancer, breast cancer, renal cancer, hepatocellular cancer, lung cancer, ovarian cancer, cervical cancer, rectum cancer, liver cancer, kidney cancer, stomach cancer, skin cancer, gastric cancer, esophageal cancer, head and neck cancer, melanoma, neuroendocrine cancers, CNS cancers (e.g., neuroblastoma), brain tumors (e.g., glioma, anaplastic oligodendroglioma, adult glioblastoma multiforme, and adult anaplastic astrocytoma), bone cancer, or soft tissue sarcoma. In some embodiments, the solid tumor is non-small cell lung cancer, small-cell lung cancer, colon cancer, CNS cancer, melanoma, ovarian cancer, renal cancer, pancreatic cancer, prostate cancer, or breast cancer.

In certain embodiments, a SYKi is administered to a patient receiving one or more anti-cancer agents selected from enzalutamide, abiraterone, abiraterone acetate, apalutamide, galeterone, olaparib, niraparib, veliparib, rucaparib, flutamide, nilutamide, bicalutamide, ketonazole, orteronel, finasteride, dutasteride, bexlosteride, izonsteride, turosteride, episteride, dexamethasone, prednisone, leuprolide, goserelin, triptorelin, histrelin, estrogen, cyproterone acetate, spironolactone, flutamide, hydroxyflutamide, docetaxel, cabazitaxel, sipuleucel-T, ODM-201, VT-464 and EPI-506, wherein the patient is suffering from myleosuppression.

In certain embodiments, a SYKi is administered to a patient receiving one or more anti-cancer agents that inhibit or modulate the activities of Bruton's tyrosine kinase, spleen tyrosine kinase, apoptosis signal-regulating kinase, Janus kinase, lysyl oxidase, lysyl oxidase-like proteins, matrix metallopeptidase, bromodomain-containing protein, adenosine A2B receptor, isocitrate dehydrogenase, serine/threonine kinase TPL2, discoidin domain receptor, serine/threonine-protein kinases, IKK, MEK, EGFR, histone deacetylase, protein kinase C, or any combination thereof. In certain embodiments, the therapeutic agent may be selected from a PI3K (including PI3Kγ, PI3Kδ, PI3Kβ, PI3Kα, and/or pan-PI3K) inhibitor, a JAK (Janus kinase, including JAK1, JAK2, and/or JAK3) inhibitor, a SYK (spleen tyrosine kinase) inhibitor, a BTK (Bruton's tyrosine kinase) inhibitor, an A2B (adenosine A2B receptor) inhibitor, an ACK (activated CDC kinase, including ACK1) inhibitor, an ASK (apoptosis signal-regulating kinase, including ASK1) inhibitor, Aurora kinase, a BRD (bromodomain-containing protein, including BRD4) inhibitor, a Bcl (B-cell CLL/lymphoma, including Bcl-1 and/or Bcl-2) inhibitor, a CAK (CDK-activating kinase) inhibitor, a CaMK (calmodulin-dependent protein kinases) inhibitor, a CDK (cyclin-dependent kinases, including CDK1, 2, 3, 4, and/or 6) inhibitor, a CK (casein kinase, including CK1 and/or CK2) inhibitor, a DDR (discoidin domain receptor, including DDR1 and/or DDR2) inhibitor, a EGFR inhibitor, a FXR (famesoid x receptor) inhibitor, a FAK (focal adhesion kinase) inhibitor, a GSK (glycogen synthase kinase) inhibitor, a HDAC (histone deacetylase) inhibitor, an IDO (indoleamine 2,3-dioxygenase) inhibitor, an IDH (isocitrate dehydrogenase, including IDH1) inhibitor, an IKK (1-Kappa-B kinase) inhibitor, a KDM5 (lysine demethylase) inhibitor, a LCK (lymphocyte-specific protein tyrosine kinase) inhibitor, a LOX (lysyl oxidase) inhibitor, a LOXL (lysyl oxidase like protein, including LOXL1, LOXL2, LOXL3, LOXL4, and/or LOXL5) inhibitor, a MTH (mut T homolog) inhibitor, a MEK (mitogen-activated protein kinase kinase) inhibitor, a matrix metalloprotease (MMP, including MMP2 and/or MMP9) inhibitor, a mitogen-activated protein kinases (MAPK) inhibitor, a PD-1 (programmed cell death protein 1) inhibitor, a PD-L1 (programmed death-ligand 1) inhibitor, a PDGF (platelet-derived growth factor) inhibitor, a phosphorylase kinase (PK) inhibitor, a PLK (polo-like kinase, including PLK1, 2, 3) inhibitor, a protein kinase (PK, including protein kinase A, B, C) inhibitor, a STK (serine/threonine kinase) inhibitor, a STAT (signal transduction and transcription) inhibitor, a serine/threonine-protein kinase inhibitor, a TBK (tank-binding kinase) inhibitor, a TLR (toll-like receptor modulators, including TLR-1, TLR-2, TLR-3, TLR-4, TLR-5, TLR-6, TLR-7, TLR-8, TLR-9, TLR-10, TLR-11, TLR-12, and/or TLR-13) inhibitor, a TK (tyrosine kinase) inhibitor, a TPL2 (serine/threonine kinase) inhibitor, a NEK9 inhibitor, an Abl inhibitor, a p38 kinase inhibitor, a PYK inhibitor, a PYK inhibitor, a c-Kit inhibitor, a NPM-ALK inhibitor, a Flt-3 inhibitor, a c-Met inhibitor, a KDR inhibitor, a TIE-2 inhibitor, a VEGFR inhibitor, a SRC inhibitor, a HCK inhibitor, a LYN inhibitor, a FYN inhibitor, a YES inhibitor, a chemotherapeutic agent, an immunotherapeutic agent, a radiotherapeutic agent, an anti-neoplastic agent, an anti-cancer agent, an anti-proliferation agent, an anti-fibrotic agent, an anti-angiogenic agent, wherein the patient is suffering from myleosuppression.

In certain embodiments, the SYKi is administered in combination with one or more additional drugs selected from corticosteroids, glucocorticoids, mineralocorticoids, hydrocortisone, dexamethasone, cortisone, prednisone, prednisolone, methylprednisolone, dexamethasone, betamethasone, triamcinolone, beclometasone, fludrocortisone, fludrocortisone acetate, deoxycorticosterone, deoxycorticosterone acetate, or aldosterone. In one embodiment, the additional drug is prednisone.

AML with 11q23/MLL abnormalities accounts for 2.8% of unselected AML and is closely associated with monocytic differentiation, and has a dismal prognosis. (Blood. 2003; 102:2395-2402). In certain embodiments, the application provides methods of treatment for treating leukemia in patients with 11q23/MLL abnormalities comprising the step of administering a SYKi, in particular Compound 1 to a patient in need thereof. In certain embodiments, the patient with 11q23/MLL abnormality is diagnosed with AML or ALL. In certain embodiments, the application provides a method for treating AML in patients with 11q23/MLL abnormalities comprising the step of administering a SYKi, in particular Compound 1:

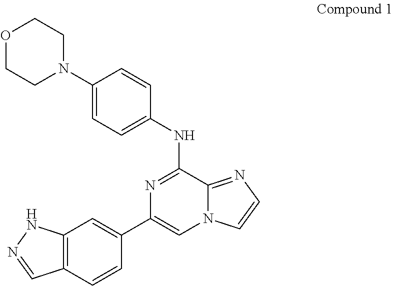 [SMILES: c1cn2cc(-c3ccc4cn[nH]c4c3)nc(Nc3ccc(N4CCOCC4)cc3)c2n1]

to a patient in need thereof. In certain embodiments, the application provides a method for testing a patient with cancer for 11q23/MLL abnormality and administering a SYKi for patients diagnosed positive or the mutation. In certain embodiments, the cancer is leukemia or lymphoma. The 11q23/MLL rearrangement can be accomplished by routine diagnostic methodology, for example the methodology described in U.S. Pat. No. 6,121,419.

In certain embodiments, the application provides methods for protecting hematopoietic cells in a patient in need thereof, comprising the step of administering a SYKi. In certain embodiments, the application provides method for treating acquired bone marrow failure in a patient in need thereof, comprising the step of administering a SYKi compound, in particular a selective SYKi, for example, Compound 1. In certain embodiments, the bone marrow failure is associated with aplastic anemia.

### Inhibitors of Spleen Tyrosine Kinases (SYKi)

In certain embodiments the following SYKi compounds can be used in the methods described herein.

The following SYKi compounds of Formula (I) are disclosed in U.S. Pat. No. 9,120,811, the contents of which are incorporated by reference herein:

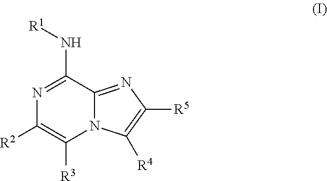

and pharmaceutically acceptable salts thereof, wherein
R¹ is phenyl substituted with one or two groups selected from
halo,
hydroxy,
carboxy,
cycloalkyl optionally substituted with one or two groups chosen from hydroxy, lower alkoxy, and lower alkyl,
heterocycloalkyl optionally substituted with one or two groups chosen from hydroxy, lower alkoxy, lower alkyl, lower alkyl substituted with hydroxy, optionally substituted amino, and oxo,
heteroaryl,
amino optionally substituted with one or two groups chosen from lower alkyl, lower alkyl substituted with halo, lower alkyl substituted with hydroxy, and lower alkyl substituted with lower alkoxy,
—C(O)NR⁶R⁷ wherein R⁶ and R⁷ are independently selected from hydrogen, lower alkyl, lower alkyl substituted with hydroxy, lower alkyl substituted with optionally substituted amino, cycloalkyl, aryl, heteroaryl, and heterocycloalkyl, or R⁶ and R⁷ together with the nitrogen to which they are bound form a 3- to 7-membered heterocycloalkyl ring optionally substituted with one or two groups chosen from hydroxy, lower alkyl, and lower alkyl substituted with hydroxy,
—S(O)₂NR⁶R⁷ wherein R⁶ and R⁷ are independently selected from hydrogen, lower alkyl, lower alkyl substituted with hydroxy, lower alkyl substituted with optionally substituted amino, cycloalkyl, aryl, heteroaryl, and heterocycloalkyl, or R⁶ and R⁷ together with the nitrogen to which they are bound form a 3- to 7-membered heterocycloalkyl ring optionally substituted with one or two groups chosen from hydroxy, lower alkyl, and lower alkyl substituted with hydroxy, provided that at least one of R₆ and R⁷ is not hydrogen,
lower alkoxy optionally substituted with one or two groups chosen from hydroxy, lower alkoxy, optionally substituted amino, carboxy, aminocarbonyl, and heterocycloalkyl,
heteroaryloxy, and
lower alkyl optionally substituted with one or two groups chosen from hydroxy, lower alkoxy, halo, trifluoromethyl, optionally substituted amino, and heterocycloalkyl optionally substituted with lower alkyl; or

### R¹ is

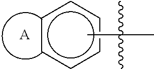

wherein A is chosen from aryl, cycloalkyl and heterocycloalkyl groups, each of which groups having from 5 to 7 ring atoms including the atoms shared with the 6 membered aromatic ring and each of which groups being optionally substituted;
R² is chosen from optionally substituted aryl and optionally substituted heteroaryl;
R³ is hydrogen;
R⁴ is hydrogen; and
R⁵ is hydrogen.

The following compounds are provided for use in the methods and compositions described herein (see, U.S. Pat. No. 9,120,811) N-(3,4-dimethoxyphenyl)-6-(3-methylphenyl)imidazo[1,2-a]pyrazin-8-amine; N-(3,4-dimethoxyphenyl)-6-(3-nitrophenyl)imidazo[1,2-a]pyrazin-8-amine; N-(3,4-dimethoxyphenyl)-6-{3-[(ethylamino)methyl]phenyl}imidazo[1,2-a]pyrazin-8-amine; N-(3,4-dimethoxyphenyl)-6-[3-(trifluoromethyl)phenyl]imidazo[1,2-a]pyrazin-8-amine; N-(3,4-dimethoxyphenyl)-6-(3-methoxyphenyl)imidazo[1,2-a]pyrazin-8-amine; N-(3,4-dimethoxyphenyl)-6-(pyridin-4-yl)imidazo[1,2-a]pyrazin-8-amine; N-(3,4-dimethoxyphenyl)-6-(pyridin-3-yl)imidazo[1,2-a]pyrazin-8-amine; N-(3,4-dimethoxyphenyl)-6-phenylimidazo[1,2-a]pyrazin-8-amine; 3-{8-[(3,4-dimethoxyphenyl)amino]imidazo[1,2-a]pyrazin-6-yl}benzonitrile; N-(3,4-dimethoxyphenyl)-6-(4-fluorophenyl)imidazo[1,2-a]pyrazin-8-amine; 4-{8-[(3,4-dimethoxyphenyl)amino]imidazo[1,2-a]pyrazin-6-yl}benzene-1-sulfonamide; N-(3,4-dimethoxyphenyl)-6-{4-[(ethylamino)methyl]phenyl}imidazo[1,2-a]pyrazin-8-amine; 6-(4-chlorophenyl)-N-(3,4-dimethoxyphenyl)imidazo[1,2-a]pyrazin-8-amine; 6-(3-chlorophenyl)-N-(4-ethoxy-3-methoxyphenyl)imidazo[1,2-a]pyrazin-8-amine; N-(3,4-dimethoxyphenyl)-6-(4-methanesulfonylphenyl)imidazo[1,2-a]pyrazin-8-amine; 4-{8-[(3,4-dimethoxyphenyl)amino]imidazo[1,2-a]pyrazin-6-yl}benzonitrile; N-(3,4-dimethoxyphenyl)-6-(4-methylphenyl)imidazo[1,2-a]pyrazin-8-amine; N-(4-ethoxy-3-methoxyphenyl)-6-(3-methylphenyl)imidazo[1,2-a]pyrazin-8-amine; N-(4-ethoxy-3-methoxyphenyl)-6-(3-fluorophenyl)imidazo[1,2-a]pyrazin-8-amine; 6-(3,4-difluorophenyl)-N-(4-ethoxy-3-methoxyphenyl)imidazo[1,2-a]pyrazin-8-amine; 6-(4-chloro-3-methylphenyl)-N-(4-ethoxy-3-methoxyphenyl)imidazo[1,2-a]pyrazin-8-amine; 3-{8-[(4-ethoxy-3-methoxyphenyl)amino]imidazo[1,2-a]pyrazin-6-yl}benzene-1-sulfonamide; N-(4-ethoxy-3-methoxyphenyl)-6-(3-methanesulfonylphenyl)imidazo[1,2-a]pyrazin-8-amine; N-(4-ethoxy-3-methoxyphenyl)-6-(4-fluoro-3-methylphenyl)imidazo[1,2-a]pyrazin-8-amine; N-(4-ethoxy-3-methoxyphenyl)-6-(3-fluoro-4-methylphenyl)imidazo[1,2-a]pyrazin-8-amine; 6-(3-chloro-4-methylphenyl)-N-(4-ethoxy-3-methoxyphenyl)imidazo[1,2-a]pyrazin-8-amine; N-(4-ethoxy-3-methoxyphenyl)-6-(2-fluoropyridin-4-yl)imidazo[1,2-a]pyrazin-8-amine; N-(4-ethoxy-3-methoxyphenyl)-6-(5-methylpyridin-3-yl)imidazo[1,2-a]pyrazin-8-amine; 6-(5-chloropyridin-3-yl)-N-(4-ethoxy-3-methoxyphenyl)imidazo[1,2-a]pyrazin-8-amine; N-(4-ethoxy-3-methoxyphenyl)-6-(pyrimidin-5-yl)imidazo[1,2-a]pyrazin-8-amine; 1-{4-[(4-{8-[(4-ethoxy-3-methoxyphenyl)amino]imidazo[1,2-a]pyrazin-6-yl}phenyl)methyl]piperazin-1-yl}ethan-1-one; 1-{4-[(3-{8-[(3,4-dimethoxyphenyl)amino]imidazo[1,2-a]pyrazin-6-yl}phenyl)methyl]piperazin-1-yl}ethan-1-one; N-(3,4-dimethoxyphenyl)-6-[3-(piperazin-1-ylmethyl)phenyl]imidazo[1,2-a]pyrazin-8-amine; N-(3,4-dimethoxyphenyl)-6-[4-(piperazin-1-ylmethyl)phenyl]imidazo[1,2-a]pyrazin-8-amine; 6-(2,3-dihydro-1,4-benzodioxin-6-yl)-N-(3,4-dimethoxyphenyl)imidazo[1,2-a]pyrazin-8-amine; N-(3-{8-[(3,4-dimethoxyphenyl)amino]imidazo[1,2-a]pyrazin-6-yl}phenyl)acetamide; 6-(3-aminophenyl)-N-(3,4-dimethoxyphenyl)imidazo[1,2-a]pyrazin-8-amine; N-(4-{8-[(3,4-dimethoxyphenyl)amino]imidazo[1,2-a]pyrazin-6-yl}phenyl)acetamide; N-(3,4-dimethoxyphenyl)-6-(thiophen-3-yl)imidazo[1,2-a]pyrazin-8-amine; N-(3,4-dimethoxyphenyl)-6-(1H-indazol-5-yl)imidazo[1,2-a]pyrazin-8-amine; N-(3,4-dimethoxyphenyl)-6-[4-(1H-imidazol-2-yl)phenyl]imidazo[1,2-a]pyrazin-8-amine; N-(3,4-dimethoxyphenyl)-6-(quinolin-6-yl)imidazo[1,2-a]pyrazin-8-amine; N-(3,4-dimethoxyphenyl)-6-(1H-indazol-6-yl)imidazo[1,2-a]pyrazin-8-amine; 6-{8-[(3,4-dimethoxyphenyl)amino]imidazo[1,2-a]pyrazin-6-yl}-3,4-dihydro-2H-1,4-benzoxazin-3-one; 6-(1,3-benzothiazol-5-yl)-N-(3,4-dimethoxyphenyl)imidazo[1,2-a]pyrazin-8-amine; 6-(1,3-benzothiazol-6-yl)-N-(3,4-dimethoxyphenyl)imidazo[1,2-a]pyrazin-8-amine; 6-{8-[(3,4-dimethoxyphenyl)amino]imidazo[1,2-a]pyrazin-6-yl}quinazolin-2-amine; N-(3,4-dimethoxyphenyl)-6-(thiophen-2-yl)imidazo[1,2-a]pyrazin-8-amine; 3-amino-5-{8-[(3,4-dimethoxyphenyl)amino]imidazo[1,2-a]pyrazin-6-yl}-1-methyl-1,2-dihydropyridin-2-one; 6-{8-[(3,4-dimethoxyphenyl)amino]imidazo[1,2-a]pyrazin-6-yl}quinolin-2-amine; 6-(4-aminophenyl)-N-(3,4-dimethoxyphenyl)imidazo[1,2-a]pyrazin-8-amine; 6-(1H-1,3-benzodiazol-5-yl)-N-(3,4-dimethoxyphenyl)imidazo[1,2-a]pyrazin-8-amine; N-(3,4-dimethoxyphenyl)-6-[3-(1H-imidazol-5-yl)phenyl]imidazo[1,2-a]pyrazin-8-amine; 7-{8-[(3,4-dimethoxyphenyl)amino]imidazo[1,2-a]pyrazin-6-yl}-3,4-dihydro-2H-1,4-benzoxazin-3-one; N-(4-ethoxy-3-methoxyphenyl)-6-(1H-indazol-5-yl)imidazo[1,2-a]pyrazin-8-amine; N-(3,4-dimethoxyphenyl)-6-[4-(1H-imidazol-5-yl)phenyl]imidazo[1,2-a]pyrazin-8-amine; N-(4-ethoxy-3-methoxyphenyl)-6-(1H-indazol-6-yl)imidazo[1,2-a]pyrazin-8-amine; 6-(1,3-benzothiazol-6-yl)-N-(4-ethoxy-3-methoxyphenyl)imidazo[1,2-a]pyrazin-8-amine; N-(3,4-dimethoxyphenyl)-6-[3-(1,3-thiazol-2-yl)phenyl]imidazo[1,2-a]pyrazin-8-amine; N-(3,4-dimethoxyphenyl)-6-(1-methyl-1H-1,3-benzodiazol-5-yl)imidazo[1,2-a]pyrazin-8-amine; 5-{8-[(3,4-dimethoxyphenyl)amino]imidazo[1,2-a]pyrazin-6-yl}-1,2-dihydropyridin-2-one; 6-(1,3-benzothiazol-5-yl)-N-(4-ethoxy-3-methoxyphenyl)imidazo[1,2-a]pyrazin-8-amine; N-(3,4-dimethoxyphenyl)-6-[4-(1,3-oxazol-2-yl)phenyl]imidazo[1,2-a]pyrazin-8-amine; (3-{[6-(1H-indazol-6-yl)imidazo[1,2-a]pyrazin-8-yl]amino}phenyl)methanol; 5-{8-[(3,4-dimethoxyphenyl)amino]imidazo[1,2-a]pyrazin-6-yl}pyridin-2-amine; N-(3,4-dimethoxyphenyl)-6-[3-(1,3-oxazol-2-yl)phenyl]imidazo[1,2-a]pyrazin-8-amine; N-[6-(1H-indazol-6-yl)imidazo[1,2-a]pyrazin-8-yl]-3,4-dihydro-2H-1,4-benzoxazin-6-amine; 1-(4-{[6-(1H-indazol-6-yl)imidazo[1,2-a]pyrazin-8-yl]amino}phenyl)ethanol; N-(3,4-dimethoxyphenyl)-6-[4-(1,3-thiazol-2-yl)phenyl]imidazo[1,2-a]pyrazin-8-amine; (5-{[6-(1H-indazol-6-yl)imidazo[1,2-a]pyrazin-8-yl]amino}-2-methoxyphenyl)methanol; N-(3,4-dimethoxyphenyl)-6-(1H-indol-6-yl)imidazo[1,2-a]pyrazin-8-amine; N-(3,4-dimethoxyphenyl)-6-(1-methyl-1H-1,3-benzodiazol-6-yl)imidazo[1,2-a]pyrazin-8-amine; N-(4-ethoxy-3-methoxyphenyl)-6-(1-methyl-1H-indazol-5-yl)imidazo[1,2-a]pyrazin-8-amine; N-(4-ethoxy-3-methoxyphenyl)-6-(1-methyl-1H-indazol-6-yl)imidazo[1,2-a]pyrazin-8-amine; N-(3,4-dimethoxyphenyl)-6-(1-methyl-1H-indazol-5-yl)imidazo[1,2-a]pyrazin-8-amine; 6-(1H-1,2,3-benzotriazol-6-yl)-N-(3,4-dimethoxyphenyl)imidazo[1,2-a]pyrazin-8-amine; N-(3,4-dimethoxyphenyl)-6-{1H-imidazo[4,5-b]pyridin-6-yl}imidazo[1,2-a]pyrazin-8-amine; 6-(1,3-benzoxazol-5-yl)-N-(3,4-dimethoxyphenyl)imidazo[1,2-a]pyrazin-8-amine; 6-(1,3-benzoxazol-6-yl)-N-(3,4-dimethoxyphenyl)imidazo[1,2-a]pyrazin-8-amine; 6-{8-[(3,4-dimethoxyphenyl)amino]imidazo[1,2-a]pyrazin-6-yl}-4-methyl-3,4-dihydro-2H-1,4-benzoxazin-3-one; N-(3,4-dimethoxyphenyl)-6-(i-methyl-1H-indazol-6-yl)imidazo[1,2-a]pyrazin-8-amine; N-(3,4-dimethoxyphenyl)-6-(1H-indol-5-yl)imidazo[1,2-a]pyrazin-8-amine; 6-{8-[(3,4-dimethoxyphenyl)amino]imidazo[1,2-a]pyrazin-6-yl}quinolin-3-amine; 2-(4-{[6-(1H-indazol-6-yl)imidazo[1,2-a]pyrazin-8-yl]amino}phenyl)propan-2-ol; 5-{8-[(3,4-dimethoxyphenyl)amino]imidazo[1,2-a]pyrazin-6-yl}-1H-indazol-3-amine; 6-{8-[(3,4-dimethoxyphenyl)amino]imidazo[1,2-a]pyrazin-6-yl}-1H-1,3-benzodiazol-2-amine; 6-{8-[(3,4-dimethoxyphenyl)amino]imidazo[1,2-a]pyrazin-6-yl}-2H,3H,4H-pyrido[3,2-b][1,4]oxazin-3-one; 6-{8-[(3,4-dimethoxyphenyl)amino]imidazo[1,2-a]pyrazin-6-yl}-2-methyl-3,4-dihydro-2H-1,4-benzoxazin-3-one; 6-{8-[(3,4-dimethoxyphenyl)amino]imidazo[1,2-a]pyrazin-6-yl}-2,2-dimethyl-3,4-dihydro-2H-1,4-benzoxazin-3-one; 7-{8-[(3,4-dimethoxyphenyl)amino]imidazo[1,2-a]pyrazin-6-yl}quinolin-2-ol; 2-(4-{[6-(1H-indazol-6-yl)imidazo[1,2-a]pyrazin-8-yl]amino}phenyl)-2-methylpropan-1-ol; 6-{8-[(3,4-dimethoxyphenyl)amino]imidazo[1,2-a]pyrazin-6-yl}-1H-indazol-3-amine; (4-{[6-(1H-indazol-6-yl)imidazo[1,2-a]pyrazin-8-yl]amino}-2-methoxyphenyl)methanol; 6-(2,3-dihydro-1H-indol-6-yl)-N-(3,4-dimethoxyphenyl)imidazo[1,2-a]pyrazin-8-amine; N-[6-(3-amino-1H-indazol-5-yl)imidazo[1,2-a]pyrazin-8-yl]-3,4-dihydro-2H-1,4-benzoxazin-6-amine; N-{4-[3-(dimethylamino)propoxy]-3-methoxyphenyl}-6-(1H-indazol-6-yl)imidazo[1,2-a]pyrazin-8-amine; 3-(4-{[6-(1H-indazol-6-yl)imidazo[1,2-a]pyrazin-8-yl]amino}-2-methoxyphenoxy)propan-1-ol; 6-(1H-indazol-6-yl)-N-[4-methoxy-3-(pyrrolidin-1-yl)phenyl]imidazo[1,2-a]pyrazin-8-amine; 5-{[6-(1H-indazol-6-yl)imidazo[1,2-a]pyrazin-8-yl]amino}-2,3-dihydro-1H-indol-2-one; 7-{8-[(3,4-dimethoxyphenyl)amino]imidazo[1,2-a]pyrazin-6-yl}quinoxalin-2-ol; 7-{8-[(3,4-dimethoxyphenyl)amino]imidazo[1,2-a]pyrazin-6-yl}-1H,2H,3H-pyrido[2,3-b][1,4]oxazin-2-one; N-[6-(1H-indazol-6-yl)imidazo[1,2-a]pyrazin-8-yl]-4-methyl-3,4-dihydro-2H-1,4-benzoxazin-7-amine; N-(2-fluoro-4-methoxyphenyl)-6-(1H-indazol-6-yl)imidazo[1,2-a]pyrazin-8-amine; 6-(1H-indazol-6-yl)-N-[3-methoxy-4-(pyrrolidin-1-yl)phenyl]imidazo[1,2-a]pyrazin-8-amine; N-[6-(1H-indazol-6-yl)imidazo[1,2-a]pyrazin-8-yl]-2,3,4,5-tetrahydro-1,5-benzoxazepin-7-amine; 1-(4-{[6-(3-amino-1H-indazol-6-yl)imidazo[1,2-a]pyrazin-8-yl]amino}phenyl)ethan-1-ol; 6-(3,4-dihydro-2H-1,4-benzoxazin-6-yl)-N-(3,4-dimethoxyphenyl)imidazo[1,2-a]pyrazin-8-amine; N-[6-(1H-indazol-6-yl)imidazo[1,2-a]pyrazin-8-yl]-4-methyl-3,4-dihydro-2H-1,4-benzoxazin-6-amine; 6-{[6-(1H-indazol-6-yl)imidazo[1,2-a]pyrazin-8-yl]amino}-2,3-dihydro-1H-indol-2-one; N-(3,4-dimethoxyphenyl)-6-{1H-pyrrolo[3,2-b]pyridin-6-yl}imidazo[1,2-a]pyrazin-8-amine; N-[6-(1H-indazol-6-yl)imidazo[1,2-a]pyrazin-8-yl]-3,4-dihydro-2H-1,4-benzoxazin-7-amine; 6-{8-[(4-ethoxy-3-methoxyphenyl)amino]imidazo[1,2-a]pyrazin-6-yl}-1H-indazol-3-amine; N-[6-(2-aminoquinazolin-6-yl)imidazo[1,2-a]pyrazin-8-yl]-4-methyl-3,4-dihydro-2H-1,4-benzoxazin-6-amine; 2-methyl-2-(4-{[6-(1-methyl-1H-1,3-benzodiazol-5-yl)imidazo[1,2-a]pyrazin-8-yl]amino}phenyl)propan-1-ol; 6-(3,4-dihydro-2H-1,4-benzoxazin-6-yl)-N-(4-ethoxy-3 methoxyphenyl)imidazo[1,2-a]pyrazin-8-amine; N-[6-(2,3-dihydro-1H-indol-6-yl)imidazo[1,2-a]pyrazin-8-yl]-3,4-dihydro-2H-1,4-benzoxazin-6-amine; (2-methoxy-5-{[6-(1-methyl-1H-1,3-benzodiazol-6-yl)imidazo[1,2-a]pyrazin-8-yl]amino}phenyl)methanol; 6-(1H-indazol-6-yl)-N-{4-[2-methyl-1-(morpholin-4-yl)propan-2-yl]phenyl}imidazo[1,2-a]pyrazin-8-amine; N-[6-(1H-indol-6-yl)imidazo[1,2-a]pyrazin-8-yl]-3,4-dihydro-2H-1,4-benzoxazin-6-amine; 7-{8-[(4-methyl-3,4-dihydro-2H-1,4-benzoxazin-7-yl)amino]imidazo[1,2-a]pyrazin-6-yl}quinoxalin-2-ol; 1-(4-{[6-(1,3-benzothiazol-5-yl)imidazo[1,2-a]pyrazin-8-yl]amino}phenyl)ethan-1-ol; 6-(1H-1,2,3-benzotnazol-6-yl)-N-[3-methoxy-4-(propan-2-yloxy)phenyl]imidazo[1,2-a]pyrazin-8-amine; 5-(8-{[3-methoxy-4-(pyrrolidin-1-yl)phenyl]amino}imidazo[1,2-a]pyrazin-6-yl)-1H-indazol-3-amine; 2-(4-{[6-(2-aminoquinazolin-6-yl)imidazo[1,2-a]pyrazin-8-yl]amino}phenyl)propan-2-ol; 6-(1H-indazol-6-yl)-N-[3-methoxy-4-(morpholin-4-yl)phenyl]imidazo[1,2-a]pyrazin-8-amine; 2-(4-{[6-(1,3-benzothiazol-5-yl)imidazo[1,2-a]pyrazin-8-yl]amino}phenyl)propan-2-ol; 6-(8-{[4-(2-hydroxypropan-2-yl)phenyl]amino}imidazo[1,2-a]pyrazin-6-yl)-3,4-dihydro-2H-1,4-benzoxazin-3-one; 6-(1H-indazol-6-yl)-N-(3-methoxyphenyl)imidazo[1,2-a]pyrazin-8-amine; 6-(1H-indazol-6-yl)-N-(4-methoxyphenyl)imidazo[1,2-a]pyrazin-8-amine; 2-(4-{[6-(1-methyl-H-1,3-benzodiazol-5-yl)imidazo[1,2-a]pyrazin-8-yl]amino}phenyl)propan-2-ol; 2-(4-{[6-(1-methyl-1H-1,3-benzodiazol-6-yl)imidazo[1,2-a]pyrazin-8-yl]amino}phenyl)propan-2-ol; 1-(4-{[6-(1H-indazol-6-yl)imidazo[1,2-a]pyrazin-8-yl]amino}-2-methoxyphenyl)piperidin-4-ol; 6-(1H-indazol-6-yl)-N-[4-(pyrrolidin-1-yl)phenyl]imidazo[1,2-a]pyrazin-8-amine; 1-(4-{[6-(1H-indazol-6-yl)imidazo[1,2-a]pyrazin-8-yl]amino}phenyl)pyrrolidin-3-ol; 2-(4-{[6-(4-methyl-3,4-dihydro-2H-1,4-benzoxazin-7-yl)imidazo[1,2-a]pyrazin-8-yl]amino}phenyl)propan-2-ol; 2-(4-{[6-(3,4-dihydro-2H-1,4-benzoxazin-6-yl)imidazo[1,2-a]pyrazin-8-yl]amino}phenyl)propan-2-ol; 2-(4-{[6-(1,4-dimethyl-1,2,3,4-tetrahydroquinoxalin-6-yl)imidazo[1,2-a]pyrazin-8-yl]amino}phenyl)propan-2-ol; 1-(4-{[6-(1H-indazol-6-yl)imidazo[1,2-a]pyrazin-8-yl]amino}-2-methoxyphenyl)azetidin-3-ol; 2-(4-{[6-(1H-indol-6-yl)imidazo[1,2-a]pyrazin-8-yl]amino}phenyl)propan-2-ol 2-(4-{[6-(3,4-dihydro-2H-1,4-benzoxazin-7-yl)imidazo[1,2-a]pyrazin-8-yl]amino}phenyl)propan-2-ol; 2-(4-{[6-(4-methyl-3,4-dihydro-2H-1,4-benzoxazin-6-yl)imidazo[1,2-a]pyrazin-8-yl]amino}phenyl)propan-2-ol; 2-(4-{[6-(2,3-dimethyl-2H-indazol-5-yl)imidazo[1,2-a]pyrazin-8-yl]amino}phenyl)propan-2-ol; 2-(4-{[6-(3-methyl-1H-indazol-5-yl)imidazo[1,2-a]pyrazin-8-yl]amino}phenyl)propan-2-ol; N-[3-methoxy-4-(morpholin-4-yl)phenyl]-6-(1-methyl-1H-1,3-benzodiazol-6-yl)imidazo[1,2-a]pyrazin-8-amine; 6-(8-{[3-methoxy-4-(morpholin-4-yl)phenyl]amino}imidazo[1,2-a]pyrazin-6-yl)quinazolin-2-amine; 1-(4-{[6-(1H-indazol-6-yl)imidazo[1,2-a]pyrazin-8-yl]amino}phenyl)azetidin-3-ol; 1-(4-{[6-(1H-indazol-6-yl)imidazo[1,2-a]pyrazin-8-yl]amino}-2-methoxyphenyl)pyrrolidin-3-ol; 6-(3,4-dihydro-2H-1,4-benzoxazin-6-yl)-N-[3-methoxy-4-(morpholin-4-yl)phenyl]imidazo[1,2-a]pyrazin-8-amine; 6-(1H-indazol-6-yl)-N-[4-(2-methoxypropan-2-yl)phenyl]imidazo[1,2-a]pyrazin-8-amine; N-(4-ethoxy-3-methoxyphenyl)-6-(1H-indol-6-yl)imidazo[1,2-a]pyrazin-8-amine; N-[3-methoxy-4-(morpholin-4-yl)phenyl]-6-(1-methyl-1H-1,3-benzodiazol-5-yl)imidazo[1,2-a]pyrazin-8-amine; N-[4-(4-ethylpiperazin-1-yl)-3-methoxyphenyl]-6-(1H-indazol-6-yl)imidazo[1,2-a]pyrazin-8-amine; and 1-(4-{[6-(1H-indazol-6-yl)imidazo[1,2-a]pyrazin-8-yl]amino}phenyl)-3-methylpiperidin-3-ol.

In one embodiment, the SYKi is Compound 1 (also referred to as entospletinib, ENTO or GS-9973) having the formula:

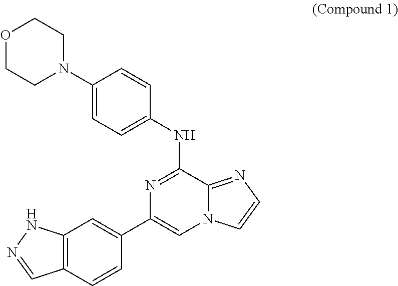 [SMILES: c1cn2cc(-c3ccc4cn[nH]c4c3)nc(Nc3ccc(N4CCOCC4)cc3)c2n1]

or a pharmaceutically acceptable salt, pharmaceutically acceptable co-crystal, pharmaceutically acceptable ester, stereoisomer, mixture of stereoisomers, or tautomer thereof. In one embodiment, the SYKi is a bis-mesylate salt of the above compound. SYKi disclosed in U.S. Pat. No. 8,455,493, U.S. Patent Publication Nos. 2015/0038504, 2015/0038505, 2015/0150881, the contents of which are incorporated by reference herein.

The following compounds are provided for use in the methods and compositions described herein (see, U.S. Pat. No. 9,290,505, the contents of which are incorporated by reference herein): 6-(6-amino-5-methylpyrazin-2-yl)-N-(4-(4-(oxetan-3-yl)piperazin-1-yl)phenyl)imidazo[1,2-a]pyrazin-8-amine; 6-(6-aminopyrazin-2-yl)-N-(4-(4-(oxetan-3-yl)piperazin-1-yl)phenyl)imidazo[1,2-a]pyrazin-8-amine; (R)-(4-(4-((6-(6-aminopyrazin-2-yl)imidazo[1,2-a]pyrazin-8-yl)amino)phenyl)morpholin-2-yl)methanol; 6-(6-aminopyrazin-2-yl)-5-methyl-N-(4-(4-(oxetan-3-yl)piperazin-1-yl)phenyl)imidazo[1,2-a]pyrazin-8-amine; 2-(5-((6-(6-aminopyrazin-2-yl)imidazo[1,2-a]pyrazin-8-yl)amino)-2-(4-(oxetan-3-yl)piperazin-1-yl)phenoxy)ethanol; and 2-((4-(4-((6-(6-aminopyrazin-2-yl)imidazo[1,2-a]pyrazin-8-yl)amino)phenyl)piperazin-1-yl)methyl)propane-1,3-diol; and 2-(5-((6-(6-amino-5-methylpyrazin-2-yl)imidazo[1,2-a]pyrazin-8-yl)amino)-2-(4-(oxetan-3-yl)piperazin-1-yl)phenoxy)ethanol.

In one embodiment, the SYKi is a compound selected from:

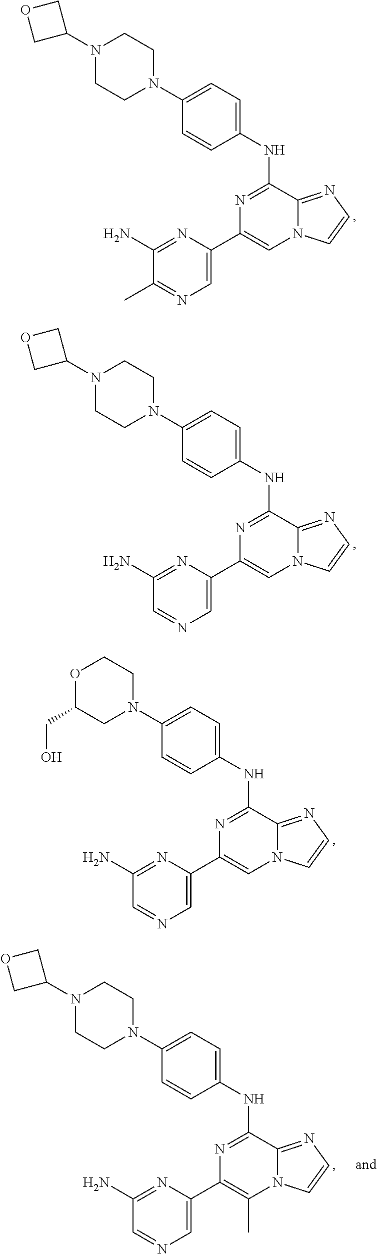 [SMILES: Cc1ncc(-c2cn3ccnc3c(Nc3ccc(N4CCN(C5COC5)CC4)cc3)n2)nc1N ; Nc1cncc(-c2cn3ccnc3c(Nc3ccc(N4CCN(C5COC5)CC4)cc3)n2)n1 ; Nc1cncc(-c2cn3ccnc3c(Nc3ccc(N4CCO[C@@H](CO)C4)cc3)n2)n1 ; Cc1c(-c2cncc(N)n2)nc(Nc2ccc(N3CCN(C4COC4)CC3)cc2)c2nccn12]

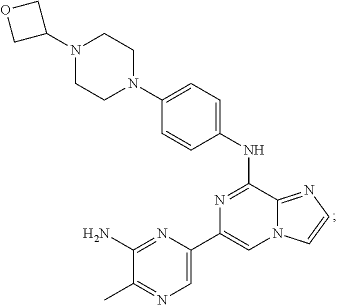 [SMILES: Cc1ncc(-c2cn3ccnc3c(Nc3ccc(N4CCN(C5COC5)CC4)cc3)n2)nc1N]

or a pharmaceutically acceptable salt, ester or derivative thereof.

The following compounds are provided for use in the methods and compositions described herein (see, U.S. Patent Application No. 2011/0152273): 6-((1R,2S)-2-Aminocyclohexylamino)-7-fluoro-4-(1-methyl-1H-pyrazol-4-yl)-1H-pyrrolo[3,4-c]pyridin-3(2H)-one; 6-((1S,2R)-2-Aminocyclohexylamino)-7-fluoro-4-(1-methyl-1H-pyrazol-4-yl)-1H-pyrrolo[3,4-c]pyridin-3(2H)-one; 6-((1R,2S)-2-Aminocyclohexylamino)-4-(1-(difluoromethyl)-1H-pyrazol-4-yl)-7-fluoro-1H-pyrrolo[3,4-c]pyridin-3(2H)-one; cis-6-(2-aminocyclohexylamino)-7-fluoro-4-(1-methyl-1H-pyrazol-4-yl)-1H-pyrrolo[3,4-c]pyridin-3(2H)-one; 6-((3R,4R)-3-Aminotetrahydro-2H-pyran-4-ylamino)-4-(1-(difluoromethyl)-1H-pyrazol-4-yl)-7-fluoro-1H-pyrrolo[3,4-c]pyridin-3(2H)-one; and 6-((3R,4R)-3-aminotetrahydro-2H-pyran-4-ylamino)-7-fluoro-4-(3-methylisothiazol-5-yl)-1H-pyrrolo[3,4-c]pyridin-3(2H)-one, or a pharmaceutically acceptable salt thereof.

The following compounds are provided for use in the methods and compositions described herein (see, U.S. Pat. No. 9,376,441, the entire content of which are incorporated by reference herein): (R)-4-((R)-1((6-(3,4-dimethoxyphenyl)pyrazolo[1,5-a]pyrazin-4-yl)oxy)ethyl)pyrrolidin-2-one; (R)-4-((R)-1-((6-(3,4-dimethoxyphenyl)-3-methylpyrazolo[1,5-a]pyrazin-4-yl)oxy)ethyl) pyrrolidin-2-one; (R)-4-((R)-1-((3-methyl-6-(4-morpholinophenyl)pyrazolo[1,5-a]pyrazin-4-yl)oxy)ethyl)pyrrolidin-2-one; (R)-4-((R)-1-((3-chloro-6-(3,4dimethoxyphenyl)pyrazolo[1,5-a]pyrazin-4-yl)oxy)ethyl)pyrrolidin-2-one; (R)-4-((R)-1-((6-(3,4-dimethoxyphenyl)-2-methylpyrazolo[1,5-a]pyrazin-4-yl)oxy)ethyl)pyrrolidin-2-one; (R)-4-((R)-1-((6-(1-(tert-butyl)-1H-pyrazol-4-yl)-3-methylpyrazolo[1,5-a]pyrazin-4-yl)oxy)ethyl)pyrrolidin-2-one; (R)-4-((R)-1-((6-(3,4-dimethoxyphenyl)pyrazolo[1,5-a]pyrazin-4-yl)oxy)ethyl)pyrrolidin-2-one; (R)-4-((R)-1((6-(3,4-dimethoxyphenyl)-3-methylpyrazolo[1,5-a]pyrazin-4-yl)oxy)ethyl)pyrrolidin-2-one; (R)-4-((R)-1-((6-(1-(tert-butyl)-1H-pyrazol-4-yl)-3-methylpyrazolo[1,5-a]pyrazin-4-yl)oxy)ethyl)pyrrolidin-2-one; (R)4-((R)-1-((3-methyl-6-(4-morpholinophenyl)pyrazolo[1,5-a]pyrazin-4-yl)oxy)ethyl)pyrrolidin-2-one; (R)-4-((R)-1-((3-chloro-6-(3,4-dimethoxyphenyl)pyrazolo[1,5-a]pyrazin-4-yl)oxy)ethyl)pyrrolidin-2-one; (R)-4-((R)-1-((6-(3,4-dimethoxyphenyl)-2-methylpyrazolo[1,5-a]pyrazin-4-yl)oxy)ethyl)pyrrolidin-2-one; (R)-4-((R)-1-(3-methyl-6-(4-morpholinophenyl)pyrazolo[1,5-a]pyrazin-4-yl)oxy)ethyl)pyrrolidin-2-one; (R)-4-((R)-1-(6-(1-tert-butyl-1H-pyrazol-4-yl)-3-methylpyrazolo[1,5-a]pyrazin-4-yloxy)ethyl)-1-((R)-1-(4-methoxyphenyl)ethyl)pyrrolidin-2-one; (R)-4-((R)-1-(3-chloro-6-(3,4dimethoxyphenyl)pyrazolo[1,5-a]pyrazin-4-yloxy)ethyl)-1-((R)-1-(4-methoxyphenyl)ethyl)pyrrolidin-2-one; (R)-4-((R)-1-(3-chloro-6-(3,4-dimethoxyphenyl)pyrazolo[1,5-a]pyrazin-4-yloxy)ethyl)pyrrolidin-2-one; (R)-4-((R)-1-(6-(3,4-dimethoxyphenyl)-2-methylpyrazolo[1,5-a]pyrazin-4-yloxy)ethyl)-1-((R)-1-(4-methoxyphenyl)pyrrolidin-2-one; (R)-4-((R)-1-(6-(3,4-dimethoxyphenyl)-2-methylprazolo[1,5-a]pyrazin-4-yloxy)ethyl)pyrrolidin-2-one; (R)-4-((R)-1-(3-bromo-6-(3,4-dimethoxyphenyl)pyrazolo[1,5-a]pyrazin-4-yloxy)ethyl)-1-((R)-1-(4-methoxyphenyl)ethyl)pyrrolidin-2-one; (R)-4-((R)-1-(3-bromo-6-(3,4-dimethoxyphenyl)pyrazolo[1,5-a]pyrazin-4-yloxy)ethyl)pyrrolidin-2-one; 4-(4-(4-((R)-1-((R)-5-oxopyrrolidin-3-yl)ethoxy)pyrazolo[1,5-a]pyrazin-6yl)phenyl)piperazine-1-carboxylate; (R)-4-((R)-1-((6-(4-(piperazin-1-yl)phenyl)pyrazolo[1,5-a]pyrazin-4-yl)oxy)ethyl)pyrrolidin-2-one; (R)-4-((R)-1-((6-(4-(4-acetylpiperazin-1-yl)phenyl)pyrazolo[1,5-a]pyrazin-4-yl)oxy)ethyl)pyrrolidin-2-one; (R)-4-((R)-1-((6-(4-(4-(methylsulfonyl)piperazin 1-yl)phenyl)pyrazolo[1,5-a]pyrazin-4-yl)oxy)ethyl)pyrrolidin-2-one; tert-butyl 4-(2-methoxy-4-(4-((R)-1-((R)-5-oxopyrrolidin-3-yl)ethoxy)pyrazolo[1,5-a]pyrazin-6-yl)phenyl)piperazine-1-carboxylate; (R)-4-((R)-1-((6-(3-methoxy-4-(piperazin-1-yl)phenyl)prazolo[1,5-a]pyrazin-4-yl)oxy)ethyl)pyrrolidin-2-one; (R)-4-((R)-1-((6-(4-(4-acetylpiperazin-1-yl)-3-methoxyphenyl)pyrazolo[1,5-a]pyrazin-4-yl)oxy)ethyl)pyrrolidin-2-one; (R)-4-((R)-1-((6-(3-methoxy-4-(4-(methylsulfonyl)piperazin-1-yl)phenyl)pyrazolo[1,5-a]pyrazin-4-yl)oxy)ethyl)pyrrolidin-2-one; tert-butyl 4-(4(3-methyl-4-((R)-1-((R)-5-oxopyrrolidin-3-yl)ethoxy)pyrazolo[1,5-a]pyrazin-6-yl)phenyl)piperazine-1-carboxylate; (R)-4-((R)-1-((3-methyl-6-(4-(piperazin-1-yl)phenyl)pyrazolo[1,5-a]pyrazin-4-yl)oxy)ethyl)pyrrolidin-2-one; (R)-4-((R)-1-((6-(4-(4-acetylpiperazin-1-yl)phenyl)-3methylpyrazolo[1,5-a]pyrazin-4-yl)oxy)ethyl)pyrrolidin-2-one; (R)-4-((R)-1-((3-methyl-6-(4-(4-(methylsulfonyl)piperazin-1-yl)phenyl)pyrazolo[1,5-a]pyrazin-4-yl)oxy)ethyl)pyrrolidin-2-one; tert-butyl 4-(2-methoxy-4-(3-methyl-4((R)-1-((R)-5-oxopyrrolidin-3-yl)ethoxy)pyrazolo[1,5-a]pyrazin-6-yl)phenyl)piperazine-1-carboxylate; (R)-4-((R)-1-((6-(3-methoxy-4-(piperazin-1-yl)phenyl)-3-methylprazolo[1,5-a]pyrazin-4-yl)oxy)ethyl)pyrrolidin-2-one; (R)-4-((R)-1-((6(4(4-acetylpiperazin-1-yl)-3-methoxyphenyl)-3-methylprazolo[1,5-a]pyrazin-4-yl)oxy)ethyl)pyrrolidin-2-one; (R)-4-((R)-1-((6-(3-methoxy-4-(4-(methylsulfonyl)piperazin-1-yl)phenyl)-3-methylpyrazolo[1,5-a]pyrazin-4-yl)oxy)ethyl)pyrrolidin-2-one; (R)-4-((R)-1-((6-(3-methoxy-4-(4-(oxetan-3-yl)piperazin-1-yl)phenyl)-3-methylpyrazolo[1,5-a]pyrazin-4-yl)oxy)ethyl)pyrrolidin-2-one; 6-chloro-4-((R)-1-((R)-1-((R)-1-(4-methoxyphenyl)ethyl)-5-oxopyrrolidin-3-yl)ethoxy)pyrazolo[1,5-a]pyrazine-3-carbonitrile; 6-(3,4-dimethoxyphenyl)-4-((R)-1-((R)-5-oxopyrrolidin-3-yl)ethoxy)pyrazolo[1,5-a]pyrazine-3-carbonitrile; 6-(4-(4-acetylpiperazin-1-yl)phenyl)-4-((R)-1-((R)-5-oxopyrrolidin-3-yl)ethoxy)pyrazolo[1,5-a]pyrazine-3-carbonitrile; 6-(4-(4-(methylsulfonyl)piperazin-1-yl)phenyl)-4-((R)-1-((R)-5-oxopyrrolidin-3-yl)ethoxy)pyrazolo[1,5-a]pyrazine-3-carbonitrile; 6-(4-(4-(oxetan-3-yl)piperazin-1-yl)phenyl)-4-((R)-1-((R)-5-oxopyrrolidin-3-yl)ethoxy)pyrazolo[1,5-]pyrazine-3-carbonitrile; (R)-4-((R)-1-(6-(3,4-dimethoxyphenyl)-3-methyl-3H-imidazo[4,5-c]pyridin-4-yloxy)ethyl)pyrrolidin-2-one; (R)-4-((R)-1-(6-(3,4-dimethoxyphenyl)-3-ethyl-3H-imidazo[4,5-c]pyridin-4-yloxy)ethyl)pyrrolidin-2-one; (R)-4-((S)-1-(6-(3,4-dimethoxyphenyl)-3-methyl-3H-imidazo[4,5-c]pyridin-4-yloxy)ethyl)pyrrolidin-2-one; (R)-4-((R)-1-(3-cyclopropyl-6-(3,4-dimethoxyphenyl)-3H-imidazo[4,5-c]pyridin-4-yloxy)ethyl)pyrrolidin-2-one; (R)-4-((R)-1-(3-(difluoromethyl)-6-(3,4-dimethoxyphenyl)-3H-imidazo[4,5-c]pyridin-4-yloxy)ethyl)pyrrolidin-2-one; (R)-4((R)-1-(3-methyl-6-(3,4,5-trimethoxyphenyl)-3H-imidazo[4,5-c]pyridin-4-yloxy)ethyl)pyrrolidin-2-one; (R)-4-((6-(3,4-dimethoxyphenyl)-3methyl-3H-imidazo[4,5-c]pyridin-4-yloxy)methyl)pyrrolidin-2-one; (R)-4-((R)-2-cyclopropyl-1-(6-(3,4-dimethoxyphenyl)-3-methyl-3H-imidazo[4,5-c]pyridin-4-yloxy)ethyl)pyrrolidin-2-one; (R)-4-((R)-1-(3-methyl-6-(4-morpholinophenyl)-3H-imidazo[4,5-c]pyridin-4-yloxy)ethyl)pyrrolidin-2-one; (R)-4-((R)-1-(6-(3dimethoxyphenyl)-3H-imidazo[4,5-c]pyridin-4-yloxy)ethyl)pyrrolidin-2-one; (R)-4-((R)-1-(6-(3,4-dimethoxyphenyl)-3-isopropyl-3H-imidazo[4,5-c]pyridin-4-yloxy)ethyl)pyrrolidin-2-one; 4-(3-methyl-4-((R)-1-((R)-5-oxopyrrolidin-3-yl)ethoxy)-3H-imidazo[4,5-c]pyridin-6-yl)benzonitrile; (4R)-4-((1R)-1-(6-(3,4-dimethoxyphenyl)-2,3-dimethyl-3a,7a-dihydro-3H-imidazo[4,5-c]pyridin-4-yloxy)ethyl)pyrrolidin-2-one; (R)-4-((R)-1-(6-(3,4-dimethoxyphenyl)-3-(2,2,2-trifluoroethyl)-3H-imidazo[4,5-c]pyridin-4-yloxy)ethyl)pyrrolidin-2-one; (R)-4-((R)-1-(6-(3,4dimethoxyphenyl)-3-(oxetan-3-yl)-3H-imidazo[4,5-c]pyridin-4-yloxy)ethyl)pyrrolidin-2-one; (R)-4-((R)-1-(3-(2,2-difluoroethyl)-6-(3,4-dimethoxyphenyl)-3H-imidazo[4,5-c]pyridin-4-yloxy)ethyl)pyrrolidin-2-one; (R)-4-((R)-1-(6-(3,4-dimethoxyphenyl)-3-(fluoromethyl)-3H-imidazo[4,5-c]pyridin-4-yloxy)ethyl)pyrrolidin-2-one; (R)-4-((R)-1-(6-(3-fluoro-4-methoxyphenyl)-3-methyl-3H-imidazo[4,5-c]pyridin-4-yloxy)ethyl)pyrrolidin-2-one; 2-methoxy-5-(3-methyl-4-((R)-5-oxopyrrolidin-3-yl)ethoxy)-3H-imidzo[4,5-c]pyridin-6-yl)benzonitrile2-methoxy-5-(3-methyl-4-((R)-1-((R)-5-oxopyrrolidin-3-yl)ethoxy)-3H-imidazo[4,5-c]pyridin-6-yl)benzonitrile; (R)-4-((R)-1-(3methyl-6-phenyl-3H-imidazo[4,5-c]pyridin-4-yloxy)ethyl)pyrrolidin-2-one; (R)-4-((R)-1-(3-methyl-6-(3morpholinophenyl)-3H-imidazo[4,5-c]pyridin-4-yloxy)ethyl)pyrrolidin-2-one; (R)-4-((R)-1-(6-(2-tert-butylthiazol-4-yl)-3methyl-3H-imidazo[4,5-c]pyridin-4-yloxy)ethyl)pyrrolidin-2-one; (R)-4-((R)-1-(3-methyl-6-(pyrazolo[1,5-a]pyridin-3-yl)-3H-imidazo[4,5-c]pyridin-4-yloxy)ethyl)pyrrolidin-2-one; (R)-4-((R)-1-(6-(2,2-difluorobenzo[d][1,3]dioxol-5-yl)-3-methyl-3H-imidazo[4,5-c]pyridin-4-yloxy)ethyl)pyrrolidin-2-one; (R)-4((R)-1-(3-methyl-6-(4-methyl-3,4-dihydro-2H-benzo[b][1,4]oxazin-6-yl)-3H-imidazo[4,5-c]pyridin-4-yloxy)ethyl)pyrrolidin-2-one; (R)-4-((R)-1-(6-(3,4-dihydro-2H-benzo[b][1,4]oxazin-6-yl)-3-methyl-3H-imidazo[4,5-c]pyridin-4-yloxy)ethyl)pyrrolidin-2-one; (R)-4-((R)-1-(6-(2-tert-butylthiazol-5-yl)-3methyl-3H-imidazo[4,5-c]pyridin-4-yloxy)ethyl)pyrrolidin-2-one; (R)-4-((R)-1-(6-cyclohexenyl-3-methyl-3H-imidazo[4,5-c]pyridin-4-yloxy)ethyl)pyrrolidin-2-one; -4-(3-methyl-4-((R)-1-((R)-5-oxopyrrolidin-3-yl)ethoxy)-3H-imidazo[4,5-c]-pyridin-6-yl)pyridin-2(1H)-one; 7-(3-methyl-4-((R)-1-((R)-5-oxopyrrolidin-3-yl)ethoxy)-3H-imidazo[4,5-c]pyridin-6-yl)-3,4-dihydrobenzo[f][1,4]oxazepin-5(2H)-one; (R)-4-((R)-1-(3methyl-6-(4-methyl-3,4-dihydro-2H-benzo[b][1,4]oxazin-7-yl)-3H-imidazo[4,5-c]pyridin-4-yloxy)ethyl)pyrrolidin-2-one; (R)-4-((R)-1 ((6-(benzo[d]thiazol-5-yl)-3-methyl-3H-imidazo[4,5-c]pyridin-4-yl)oxy)ethyl)pyrrolidin-2-one; (R)-4-((R)-1-((3-methyl-6-(2-methylbenzo[d]thiazol-5-yl)-3H-imidazo[4,5-c]pyridin-4-yl)oxy)ethyl)pyrrolidin-2-one; (R)-4-((R)-1-((3-methyl-6-methyl-1H-indazol-5-yl)-3H-imidazo[4,5-c]pyridin-4-yl)oxy)ethyl)pyrrolidin-2-one; (R)-4-((R)-1-((3-methyl-6-(1-methyl-1H-indazol-6-yl)-3H-imidazo[4,5-c]pyridin-4-yl)oxy)ethyl)pyrrolidin-2-one; (R)-4-((R)-1-((6-(1,3-dimethyl-1H-indazol-5-yl)-3-H-imidazo[4,5-c]pyridin-4-yl)oxy)ethyl)pyrrolidin-2-one; (R)-4-((R)-1-((3-methyl-6-(4-(4-(methylsulfonyl)piperazin-1-yl)phenyl)-3H-imidazo[4,5-c]pyridin-4-yl)oxy)ethyl)pyrrolidin-2-one; (R)-4-((R)-1-((6-(3,4-dimethoxyphenyl)3-methyl-3H-imidazo[4,5-c]pyridin-4-yl)oxy)propyl)pyrrolidin-2-one; (R)-4-((S)-1-((6-(3,4-dimethoxyphenyl)-3-methyl-3H-imidazo[4,5-c]pyridin-4-yl)oxy)-2,2,2-trifluoroethyl)pyrrolidin-2-one; (R)-4-((R)-1-((3-methyl-6-(4-(4-(oxetan-3-yl)piperazin-1-yl)phenyl)-3H-imidazo[4,5-c]pyridin-4-yl)oxy)ethyl)pyrrolidin-2-one; (R)-4-((R)-1-((6-(4-(4-acetylpiperazin-1-yl)phenyl)-3methyl-3H-imidazo[4,5-c]pyridin-4-yl)oxy)ethyl)pyrrolidin-2-one; (R)-7-((R)-1-((3-(difluoromethyl)-6-(3,4-dimethoxyphenyl)-3H-imidazo[4,5-c]pyridin-4-yl)oxy)ethyl)-5-azaspiro[2,4]heptan-4-one; N,N-dimethyl-4-(3-methyl-4-((R)-1-((R)-5-oxopyrrolidin-3-yl)ethoxy)-3H-imidazo[4,5-c]pyridin-6-yl)benzenesulfonamide; (R)-4-((R)-1-((3-(difluoromethyl)-6-(3,4-dimethoxyphenyl)-2-methyl-3H-imidazo[4,5-c]pyridin-4-yl)oxy)ethyl)pyrrolidin-2-one; (R)-4-((R)-1-((3-cyclopropyl-6-(3,4-dimethoxyphenyl)-2-methyl-3H-imidazo[4,5-c]pyridin-4-yl)oxy)ethyl)pyrrolidin-2-one; and (R)-4-((R)-1-((6-(3,4-dimethoxyphenyl)-3-isopropyl-2-methyl-3-H-imidazo[4,5-c]pyridin-4-yl)oxy)ethyl)pyrrolidin-2-one; (R)-4-((R)-1-((5-(tert-butyl)-1H-pyrazol-4-yl)-1-methyl-1H-benzo[d]imidazol-7-yl)oxy)ethyl)pyrrolidin-2-one; (R)-4((R)-1-((1-methyl-5-(6-(trifluoromethyl)pyridin-2-yl)-1H-benzo[d]imidazol-7-yl)oxy)ethyl)pyrrolidin-2-one; (R)-4-((R)-1-((5-(6-methoxypyridin-2-yl)-1-methyl-1H-benzo[d]imidazol-7-yl)oxy)ethyl)pyrrolidin-2-one; (R)-4-((R)-1-((5-(5,6-dimethoxypyridin-2-yl)-1,2-dimethyl-1H-benzo[d]-imidazol-7-yl)oxy)ethyl)pyrrolidin-2-one; (R)-4-((R)-1-((5-(5,6-dimethoxypyridin-2-yl)-1-methyl-1H-benzo[d]imidazol-7-yl)oxy)ethyl)pyrrolidin-2-one; (R)-4-((R)-1-((5-(3,4-dimethoxyphenyl)-1-methyl-1H-benzo[d]imidazol-7-yl)oxy)ethyl)pyrrolidin-2-one; (R)-4-((R)-1-((5-(6-aminopyridin-2-yl)-1-cyclopropyl-1H-benzo[d]imidazol-7-yl)oxy)ethyl)pyrrolidin-2-one; (R)-4-((R)-1-((1-cyclopropyl-5-(5-(4-morpholinopiperidin-1-yl)pyridin-2-yl)-1H-benzo[d]imidazol-7-yl)oxy)ethyl)pyrrolidin-2-one; (R)-4-((R)-1-(1-cyclopropyl-5-(5-(4-(oxetan-3-yl)piperidin-1-yl)pyridin-2-yl)-1H-benzo[d]imidazol-7-yl)oxy)ethyl)pyrrolidin-2-one; (4R)-4-((1R)-1-((5-(5-(6-oxa-3-azabicyclo[3.1.1]heptan-3-yl)pyridin-2yl)-1-cyclopropyl-1H-benzo[d]imidazol-7-yl)oxy)ethyl)pyrrolidin-2-one; (R)-4-((R)-1-((1-cyclopropyl-5-(1-methyl-1H-pyrazol-4-yl)-1H-benzo[d]imidazol-7-yl)oxy)ethyl)pyrrolidin-2-one; (R)-4-((R)-1-((1-cyclopropyl-5-(1-(oxetan-3-yl)-1H-pyrazol-4-yl)-1H-benzo[d]imidazol-7-yl)oxy)ethyl)pyrrolidin-2-one; (R)-4-((R)-1-((5-(1-(tert-butyl)-1H-pyrazol-4-yl)-1-cyclopropyl-1H-benzo[d]imidazol-7-yl)oxy)ethyl)pyrrolidin-2-one; (R)-4-((R)-1-((1-cyclopropyl-5-(1-(pyridin-4-yl)-1H-pyrazol-4-yl)-1H-benzo[d]imidazol-7-yl)oxy)ethyl)pyrrolidin-2-one; (R)-4-((R)-1-((1 cyclopropyl-5-(1-(1-hydroxy-2-methylpropan-2-yl)-1H-pyrazol-4-yl)-1H-benzo[d]imidazol-7-yl)oxy)ethyl)pyrrolidin-2-one; (R)-4-((R)-1-((1-cyclopropyl-5-(1-(2-hydroxy-2-methylpropyl)-1H-pyrazol-4-yl)-1H-benzo[d]imidazol-7-yl)oxy)ethyl)pyrrolidin-2-one; (R)-4-((R)-1-((1-cyclopropyl-5-(1-(1-(oxetan-3-yl)piperidin-4-yl)-1H-pyrazol-4-yl)-1H-benzo[d]imidazol-7-yl)oxy)ethyl)pyrrolidin-2-one; (R)-4-((R)-1-((1-cyclopropyl-5-(3-fluoro-4-(4-(oxetan-3-yl)piperazin-1-yl)phenyl)-1H-benzo[d]imidazol-7-yl)oxy)ethyl)pyrrolidin-2-one; 5-(1-cyclopropyl-7-((R)-1-((R)-5-oxopyrrolidin-3-yl)ethoxy)-1H-benzo[d]imidazol-5-yl)-2-(4-(oxetan-3-yl)piperazin-1-yl)benzonitrile; (R)-4-((R)-1-((1-cyclopropyl-5-(5-(4-(oxetan-3-yl)piperazin-1yl)pyridin-2-yl)-1H-benzo[d]imidazol-7-yl)oxy)ethyl)pyrrolidin-2-one; (R)-4-((R)-1-((1 cyclopropyl-5-(3,4-dihydro-2H-pyrido[3,2-b][1,4]oxazin-6-yl)-1H-benzo[d]imidazol-7-yl)oxy)ethyl)pyrrolidin-2-one; (R)-4-((R)-1-((1-cyclopropyl-5-(3,4-dihydro-2H-benzo[b][1,4]oxazin-6-yl)-1H-benzo[d]imidazol-7-yl)oxy)ethyl)pyrrolidin-2-one; (R)-4-((R)-1-((1-cyclopropyl-5-(6-(4-(oxetan-3-yl)piperazin-1-yl)pyridazin-3-yl)-1-H-benzo[d]imidazol-7-yl)oxy)ethyl)pyrrolidin-2-one; (R)-4-((R)-1-((1-cyclopropyl-5-(3,3-dimethylindolin-6-yl) 1H-benzo[d]imidazol-7-yl)oxy)ethyl)pyrrolidin-2-one; (R)-4-((R)-1-((1-cyclopropyl-5-(pyrimidin-4-yl)-1H-benzo[d]imidazol-7-yl)oxy)ethyl)pyrrolidin-2-one; (R)-4-((R)-1-((1-cyclopropyl-5-phenyl-1H-henzo[d]imidazol-7-yl)oxy)ethyl)pyrrolidin-2-one; (R)-4-((R)-1-((1-cyclopropyl-5-(pyrazin-2-yl)-1H-benzo[d]imidazol-7-yl)oxy)ethyl)pyrrolidin-2-one; (R)-4-((R)-1-((1-cyclopropyl-5-(pyridin-2-yl)-1H-benzo[d]imidazol-7-yl)oxy)ethyl)pyrrolidin-2-one; (R)-4-((R)-1-((1-cyclopropyl-5-(1-methyl-1,2,3,4-tetrahydroquinoxalin-6-yl)-1H-benzo[d]imidazol-7-yl)oxy)ethyl)pyrrolidin-2-one; (R)-4-((R)-1-((1-cyclopropyl-5-(1-methyl-1-H-indazol-5-yl)-1H-benzo[d]imidazol-7-yl)oxy)ethyl)pyrrolidin-2-one; (R)-4-((R)-1-((1-cyclopropyl-5-(4-(difluoromethoxy)-3-methoxyphenyl)-1H-benzo[d]imidazol-7-yl)oxy)ethyl)pyrrolidin-2-one; (R)-4-((R)-1-((1-cyclopropyl-5-(thiazol-2-yl)-1H-benzo[d]imidazol-7-yl)oxy)ethyl)pyrrolidin-2-one; (R)-4-((R)-1-((1-cyclopropyl-5-(2-(2-hydroxypropan-2-yl)thiazol-5-yl)-1H-benzo[d]imidazol-7-yl)oxy)ethyl)pyrrolidin-2-one; (R)-4-((R)-1-((1-cyclopropyl-5-(2-methylthiazol-4yl)-1H-benzo[d]imidazol-7-yl)oxy)ethyl)pyrrolidin-2-one; (R)-4-((R)-1-((1-cyclopropyl-5-(1-(tetrahydro-2H-pyran-4-yl)-1H pyrazol-5-yl)-1H-benzo[d]imidazol-7-yl)oxy)ethyl)pyrrolidin-2-one; (R)-4-((R)-1-((1-cyclopropyl-5-(4,5-dimethylthiazol-2-yl)-1H-benzo[d]imidazol-7-yl)oxy)ethyl)pyrrolidin-2-one; (R)-4-((R)-1-((5-(4-(tert-butyl)thiazol-2-yl)-1-cyclopropyl-1H-benzo[d]imidazol-7-yl)oxy)ethyl)pyrrolidin-2-one; (R)-4-((R)-1-((1-cyclopropyl-5-(2-(tetrahydro-2H-pyran-4-yl)thiazol-5-yl)-1H-benzo[d]imidazol-7-yl)oxy)ethyl)pyrrolidin-2-one; (R)-4-((R)-1-((1-cyclopropyl-5-(1,5-dimethyl-1H-pyrazol-3-yl)-1H-benzo[d]imidazol-7-yl)oxy)ethyl)pyrrolidin-2-one; (R)-4-((R)-1-((1-cyclopropyl-5-(1,2-dimethyl-1H-imidazol-4-yl)-1H-benzo[d]imidazol-7-yl)oxy)ethyl)pyrrolidin-2-one; (R)-4-((R)-1-((1-cyclopropyl-5-(5-methyl-1,3,4-thiadiazol-2-yl)-1H-benzo[d]imidazol-7-yl)oxy)ethyl)pyrrolidin-2-one; (R)-4-((R)-1((5-(1-(tert-butyl)-1H-pyrazol-3-yl)-1-cyclopropyl-1H-benzo[d]imidazol-7-yl)oxy)ethyl)pyrrolidin-2-one; (R)-4-((R)-1-((1-cyclopropyl-5-(2-methyl-1H-imidazol-5-yl)-1H-benzo[d]imidazol-7-yl)oxy)ethyl)pyrrolidin-2-one; (R)-4-((R)-1-((1-cyclopropyl-5-(5-methylthiazol-2-yl)-1H-benzo[d]imidazol-7-yl)oxy)ethyl)pyrrolidin-2-one; (R)-4-((R)-1-((1-cyclopropyl-5-(1-methyl-1H-pyrazol-3-yl)-1H-benzo[d]imidazol-7-yl)oxy)ethyl)pyrrolidin-2-one; and (R)-4-((R)-1-((1-cyclopropyl-5-(2-methyl-2H-1,2,3, -triazol-4-yl)-1H-benzo[d]imidazol-7-yl)oxy)ethyl)pyrrolidin-2-one; (R)-4-((R)-1-((5-(1-cyclobutyl-1H-pyrazol-4-yl)benzo[d]thiazol-7-yl)oxy)ethyl)pyrrolidin-2-one; (R)-4-((R)-1-((5-(1-(2,2-difluoroethyl)-1H-pyrazol-4-yl)benzo[d]thiazol-7-yl)oxy)ethyl)pyrrolidin-2-one; (R)-4-((R)-1-((-5-(1-isopropyl-1H-pyrazol-4-yl)benzo[d]thiazol-7-yl)oxy)ethyl)pyrrolidin-2-one; (R)-4-((R)-1-((5-(1-(tert-butyl)-1H-pyrazol-4-yl)benzo[d]thiazol-7-yl)oxy)ethyl)pyrrolidin-2-one; (R)-4-((R)-1-((5-(1-ethyl-1H-pyrazol-3-yl)benzol[d]thiazol-7-yl)oxy)ethyl)pyrrolidin-2-one; (R)-4-((R)-1-((5-(1-(tert-butyl)-1H-pyrazol-4-yl)-2-methylbenzo[d]thiazol-7-yl)oxy)ethyl)pyrrolidin-2-one; (R)-4-((R)-1-((5-(1-isopropyl-1H-pyrazol-3-yl)benzo[d]thiazol-7-yl)oxy)ethyl)pyrrolidin-2-one; (R)-4-((R)-1-((5-(1-(tetrahydro-2H-pyran-4-yl)-1H-pyrazol-4-yl)benzo[d]thiazol-7-yl)oxy)ethyl)pyrrolidin-2-one; (R)-4-((R)-1-((5-(5-morpholinopyridin-2-yl)benzo[d]thiazol-7-yl)oxy)ethyl)pyrrolidin-2-one; tert-butyl 4-(6-(7-((R)-1-((R)-5-oxopyrrolidin-3-yl)ethoxy)benzo[d]thiazol-5-yl)pyridin-3-yl)piperazine-1-carboxylate; (R)-4-((R)-1-((5-(5-(4-(oxetan-3-yl)piperazin-1-yl)pyridin-2-yl)benzo[d]thiazol-7-yl)oxy)ethyl)pyrrolidin-2-one; (R)-4-((R)-1-((5-(5-(4-acetylpiperazin-1-yl)pyridin-2-yl)benzo[d]thiazol-7-yl)oxy)ethyl)pyrrolidin-2-one; (R)-4-((R)-1-((5-(5-(4-(tetrahydro-2H-pyran-4-yl)piperazin-1-yl)pyridin-2-yl)benzo[d]thiazol-7-yl)oxy)ethyl)pyrrolidin-2-one; (R)-4-((R)-1-((5-(5,6-dimethoxypyridin-2-yl)benzo[d]thiazol-7-yl)oxy)ethyl)pyrrolidin-2-one; (R)-4-((R)-1-((5-(4-(4-(oxetan-3-yl)piperazin-1-yl)phenyl)benzo[d]thiazol-7-yl)oxy)ethyl)pyrrolidin-2-one; (R)-4-((R)-1-((5-(4-morpholinophenyl)benzo[d]thiazol-7-yl)oxy)ethyl)pyrrolidin-2-one; (R)-4-((R)-1-((5-(3,4-dimethoxyphenyl)benzo[d]thiazol-7-yl)oxy)ethyl)pyrrolidin-2-one; (R)-4-((R)-1-((5-(3,4-dimethoxyphenyl)-2-methylbenzo[d]thiazol-7-yl)oxy)ethyl)pyrrolidin-2-one; tert-butyl 4-(4-(7-((R)-1-((R)-5-oxopyrrolidin-3-yl)ethoxy)benzo[d]thiazol-5-yl)phenyl)piperazine-1-carboxylate; (R)-4-((R)-1-([4,5′-bibenzo[d]thiazol]-7′-yloxy)ethyl)pyrrolidin-2-one, (S)-4-((S)-1-((5-(2-(tert-butyl)thiazol-5-yl)benzo[d]thiazol-7-yl)oxy)ethyl)pyrrolidin-2-one; and (R)-4-((R)-1-((5-(1methyl-1H-thieno[3,2-c]pyrazol-5-yl)benzo[d]thiazol-7-yl)oxy)ethyl)pyrrolidin-2-one; (R)-4-((R)-1-(6-(benzo[d]thiazol-4-yl)-3-methyl-3H-imidazo[4,5-c]pyridin-4-yl)oxy)ethyl)pyrrolidin-2-one; (R)-4-((R)-1-((6-(benzo[d]thiazol-5-yl)-3-methyl-3H-imidazo[4,5-c]pyridin-4-yl)oxy)ethyl)pyrrolidin-2-one; and (R)-4-((R)-1-((3-methyl-6-(2-methylbenzo[d]thiazol-5-yl)-3H-imidazo[4,5-c-]pyridin-4-yl)oxy)ethyl)pyrrolidin-2-one.

The following compounds are provided for use in the methods and compositions described herein (see, U.S. Pat. No. 9,359,375): 4-((1R,2S)-2-aminocyclohexylamino)-2-(m-tolylamino)benzamide; 4-((1R,2S)-2-aminocyclohexylamino)-2-(1-methyl-1H-indol-4-ylamino)benzamide; 4-((1R,2S)-2-aminocyclohexylamino)-2-(4-fluorophenylamino)benzamide; 4-((1R,2S)-2-aminocyclohexylamino)-2-(3,5-dimethylphenylamino)benzamide; 2-(3-(2H-1,2,3-triazol-2-yl)phenylamino)-4-((1R,2S)-2-aminocyclohexylamino)benzamide; 4-((1R,2S)-2-aminocyclohexylamino)-2-(3-methylisothiazol-5-ylamino)benzamide; 4-((1R,2S)-2-aminocyclohexylamino)-2-(3-phenylisoxazol-5-ylamino)benzamide; 4-((1R,2S)-2-aminocyclohexylamino)-2-(1-methyl-1H-pyrazol-4-ylamino)benzamide; (R)-4-(1-amino-4-methyl-1-oxopentan-2-ylamino)-2-(3-methylisothiazol-5-yl-amino)benzamide; (R)-4-(1-amino-1-oxopropan-2-ylamino)-2-(3-methylisothiazol-5-ylamino)benzamide; (R)-4-(1-amino-3-methyl-1-oxobutan-2-ylamino)-2-(3-methylisothiazol-5-ylamino)benzamide; (R)-4-(1-amino-4-methyl-1-oxopentan-2-ylamino)-2-(5-methylisoxazol-3-ylamino)benzamide; (R)-4-(1-amino-4-methyl-1-oxopentan-2-ylamino)-2-(3-methylisoxazol-5-ylamino)benzamide; (R)-4-(1-amino-1-oxo-3-phenylpropan-2-ylamino)-2-(3-methylisothiazol-5-ylamino)benzamide; (R)-4-(1-amino-3-(benzyloxy)-1-oxopropan-2-ylamino)-2-(3-methylisothiazol-5-ylamino)benzamide; (R)-4-(1-amino-3-hydroxy-1-oxopropan-2-ylamino)-2-(3-methylisothiazol-5-ylamino)benzamide; (R)-4-(1-amino-3-cyclohexyl-1-oxopropan-2-ylamino)-2-(3-methylisothiazol-5-ylamino)benzamide; (R)-4-(2-amino-2-oxo-1-phenylethylamino)-2-(3-methylisothiazol-5-ylamino)benzamide; (R)-4-(1-amino-4-methyl-1-oxopentan-2-ylamino)-2-fluoro-6-(3-methylisothiazol-5-ylamino)benzamide; (R)-4-(i-amino-4-methyl-1-oxopentan-2-ylamino)-5-fluoro-2-(3-methylisothiazol-5-ylamino)benzamide; 4-((1R,2S)-2-aminocyclohexylamino)-5-fluoro-2-(3-methylisothiazol-5-ylamino)benzamide; (R)-4-(2-amino-1-cyclohexyl-2-oxoethylamino)-2-(3-methylisothiazol-5-ylamino)benzamide; (R)-4-(1-amino-4-methyl-1-oxopentan-2-ylamino)-3-fluoro-2-(3-methylisothiazol-5-ylamino)benzamide; (R)-4-(i-amino-1-oxo-3-phenylpropan-2-ylamino)-5-fluoro-2-(3-methylisothiazol-5-ylamino)benzamide; (R)-4-(1-amino-3-cyclopropyl-1-oxopropan-2-ylamino)-5-fluoro-2-(3-methylisothiazol-5-ylamino)benzamide; (R)-4-(1-amino-1-oxobutan-2-ylamino)-5-fluoro-2-(3-methylisothiazol-5-ylamino)benzamide; (R)-4-(1-amino-3-(4-fluorophenyl)-1-oxopropan-2-ylamino)-5-fluoro-2-(3-methylisothiazol-5-ylamino)benzamide; (R)-4-(1-amino-3-(4-hydroxyphenyl)-1-oxopropan-2-ylamino)-5-fluoro-2-(3-methylisothiazol-5-ylamino)benzamide; (R)-4-(2-amino-3-methoxypropylamino)-2-(3-methylisothiazol-5-ylamino)benzamide; (S)-4-(2-aminobutylamino)-2-(3-methylisothiazol-5-ylamino)benzamide; (S)-4-(2-aminopropylamino)-2-(3-methylisothiazol-5-ylamino)benzamide; (S)-4-(2-amino-4-methylpentylamino)-2-(3-methylisothiazol-5-ylamino)benzamide; (R)-4-(1-amino-1-oxobutan-2-ylamino)-2-(3-methylisothiazol-5-ylamino-)benzamide; (R)-4-(1-amino-1-oxobutan-2-ylamino)-2-(3-methylisothiazol-5-ylamino)benzamide; (R)-4-(1-amino-3-cyclopropyl-1-oxopropan-2-ylamino)-2-(3-methylisothiazol-5-ylamino)benzamide; (R)-4-(1-amino-3-(4-methoxyphenyl)-1-oxopropan-2-ylamino)-5-fluoro-2-(3-methylisothiazol-5-ylamino)benzamide; (R)-4-(1-amino-3-(3-fluorophenyl)-1-oxopropan-2-ylamino)-5-fluoro-2-(3-methylisothiazol-5-ylamino)benzamide; (R)-4-(1-amino-1-oxo-3-(pyridin-4-yepropan-2-ylamino)-5-fluoro-2-(3-methylisothiazol-5-ylamino)benzamide; (R)-4-(1-amino-1-oxo-3-(pyridin-3-yl)propan-2-ylamino)-5-fluoro-2-(3-methylisothiazol-5-ylamino)benzamide; (R)-4-(1-amino-3-methoxy-1-oxopropan-2-ylamino)-5-fluoro-2-(3-methylisothiazol-5-ylamino)benzamide; 6-(cis-2-aminocyclohexylamino)-4-(3-toluidino)nicotinamide; 6-(cyclopentylamino)-4-(4-morpholinophenylamino)nicotinamide; 4-(4-carbamoylphenylamino)-6-(cyclopentylamino)nicotinamide; 4-((1R,2S)-2-aminocyclohexylamino)-5-fluoro-2-(3-phenylisoxazol-5-ylamino)benzamide; 4-((1R,2S)-2-aminocyclohexylamino)-2-(3-methylisoxazol-5-ylamino)benzamide; 4-((1R,2S)-2-aminocyclohexylamino)-5-fluoro-2-(3-methylisoxazol-5-ylamino)benzamide; (R)-4-(1-amino-3-(2-fluorophenyl)-1-oxopropan-2-ylamino)-5-fluoro-2-(3-methylisothiazol-5-ylamino)benzamide; 4-(3-(2H-1,2,3-triazol-2-yl)phenylamino)-6-((1R,2S)-2-aminocyclohexylamino)nicotinamide; 6-((1R,2S)-2-aminocyclohexylamino)-4-(3-methylisothiazol-5-ylamino)nicotinamide; (R)-4-(2-amino-1-cyclopropyl-2-oxoethylamino)-5-fluoro-2-(3-methylisothiazol-5-ylamino)benzamide; 2-(3-methylisothiazol-5-ylamino)-4-(2-oxoazepan-3-ylamino)benzamide; (R)-4-(1-amino-3-(1H-indol-3-yl)-1-oxopropan-2-ylamino)-2-(3-methylisothiazol-5-ylamino)benzamide; (R)-4-(1-amino-1-oxo-3-(pyridin-2-yl)propan-2-ylamino)-5-fluoro-2-(3-methylisothiazol-5-ylamino)benzamide; 4-(1-amino-4,4,4-trifluoro-1-oxobutan-2-ylamino)-5-fluoro-2-(3-methylisothiazol-5-ylamino)benzamide; (R)-4-(i-amino-1-oxo-3-(thiophen-2-yl)propan-2-ylamino)-2-(3-methylisothiazol-5-ylamino)benzamide; (R)-4-(1-amino-1-oxo-3-(4-(pyridin-4-yl)phenyl)propan-2-ylamino)-5-fluoro-2-(3-methylisothiazol-5-ylamino)benzamide; (R)-4-(3-(4-(1H-imidazol-1-yl)phenyl)-1-amino-1-oxopropan-2-ylamino)-5-fluoro-2-(3-methylisothiazol-5-ylamino)benzamide; (R)-4-(1-amino-1-oxo-3-(4-(2-oxopyridin-1 (2H)-yl)phenyl)propan-2-ylamino)-5-fluoro-2-(3-methylisothiazol-5-ylamino)benzamide; (R)-4-(1-amino-3-(4-(methylsulfonyl)phenyl)-1-oxopropan-2-ylamino)-5-fluoro-2-(3-methylisothiazol-5-ylamino)benzamide; (R)-4-(1-amino-1-oxo-3-(4-(pyridin-3-yl)phenyl)propan-2-ylamino)-5-fluoro-2-(3-methylisothiazol-5-ylamino)benzamide; (R)-4-(i-amino-3-cyclopropyl-1-oxopropan-2-ylamino)-5-fluoro-2-(3-phenylisoxazol-5-ylamino)benzamide; 6-((1R,2S)-2-aminocyclohexylamino)-4-(pyrazolo[1,5-a]pyridin-3-ylamino)nicotinamide; and 6-((1R,2S)-2-aminocyclohexylamino)-4-(thieno[2,3-b]pyridin-3-ylamino)nicotinamide.

In one embodiment, the SYKi is a compound having the formula:

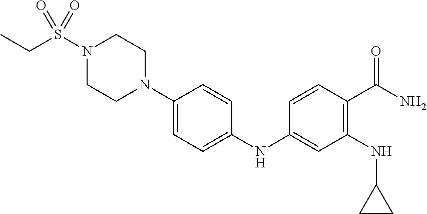 [SMILES: CCS(=O)(=O)N1CCN(c2ccc(Nc3ccc(C(N)=O)c(NC4CC4)c3)cc2)CC1]

or a pharmaceutically acceptable salt, pharmaceutically acceptable co-crystal, pharmaceutically acceptable ester, stereoisomer, mixture of stereoisomers, or tautomer thereof.

The following compounds are provided for use in the methods and compositions described herein (see, U.S. Pat. Nos. 7,435,814 and 8,227,455):

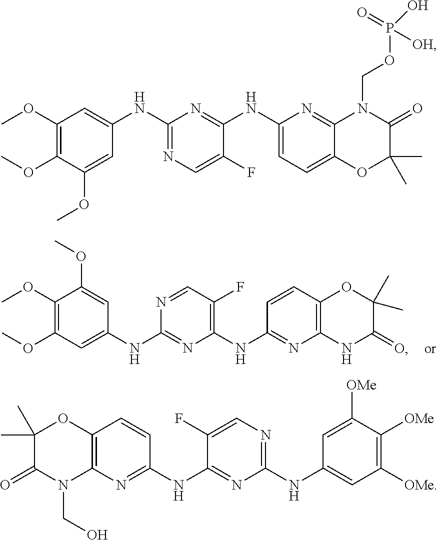

The following compounds are provided for use in the methods and compositions described herein (see, U.S. Pat. No. 8,470,835):

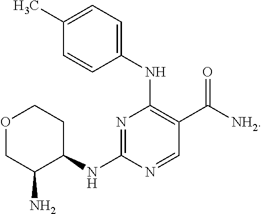 [SMILES: Cc1ccc(Nc2nc(N[C@@H]3CCOC[C@@H]3N)ncc2C(N)=O)cc1]

The following SYKi compound (CC-509) is disclosed in Ferguson et. al. (PLOS ONE, January 2016) and U.S. Pat. No. 8,470,835:

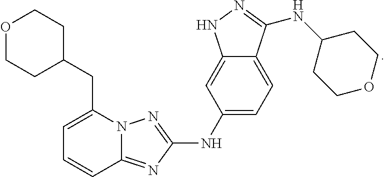 [SMILES: c1cc(CC2CCOCC2)n2nc(Nc3ccc4c(NC5CCOCC5)n[nH]c4c3)nc2c1]

The following pyridopyrazine SYKi compounds are provided for use in the methods and compositions described herein (see, U.S. Patent Publication Nos. 2016/0002221 and 2015/0307491, the content of which are incorporated by reference herein: (2S)-2-[[[7-[4-(1-acetyl-3-azetidinyl)phenyl]pyrido[3,4-b]pyrazin-5-yl]oxy]methyl]-4-morpholinecarboxylic acid 1,1-dimethylethyl ester; (2S)-2-[[[7-[4-(3-azetidinyl)phenyl]pyrido[3,4-b]pyrazin-5-yl]oxy]methyl]-4-morpholinecarboxylic acid 1,1-dimethylethyl ester; N-[[(2S)-2-[[[7-(3,4-dimethoxyphenyl)pyrido[3,4-b]pyrazin-5-yl]oxy]methyl]-4-morpholinyl]sulfonyl]-carbamic acid 1,1-dimethylethyl ester; (2S)-2-[[[7-(3,4-dimethoxyphenyl)pyrido[3,4-b]pyrazin-5-yl]oxy]methyl]4-morpholinesulfonyl isocyanate; 5-[(2S)-2-morpholinylmethoxy]-7-[4-(tetrahydro-2H-pyran-4-yl)phenyl]-pyrido[3,4-b]pyrazine; 1-[4-[5-[[(2S)-4-(methylsulfonyl)-2-morpholinyl]methoxy]pyrido[3,4-b]pyrazin-7-yl]phenyl]-ethanone; (4S)-4-[[[7-[4-(dimethylamino)phenyl]pyrido[3,4-b]pyrazin-5-yl]oxy]methyl]-1-[(1 S)-1-phenylethyl]-2-pyrrolidinone; 7-(3,4-dimethoxyphenyl)-5-[(2S)-2-morpholinylmethoxy]-pyrido[3,4-b]pyrazine; (2S)-2-[[[7-(3,4-dimethoxyphenyl)pyrido[3,4-b]pyrazin-5-yl]oxy]methyl]-4-morpholinecarboxylic acid 1,1-dimethylethyl ester; (2S)-2-[[[7-[4-(dimethylamino)phenyl]pyrido[3,4-b]pyrazin-5-yl]oxy]methyl]-4-morpholineethanamine; 2-[2-[(2S)-2-[[[7-[4-(dimethylamino)phenyl]pyrido[3,4-b]pyrazin-5-yl]oxy]methyl]-4-morpholinyl]ethyl]-1H-isoindole-1,3(2H)-dione; 4-[5-[[(2S)-4-(ethenylsulfonyl)-2-morpholinyl]methoxy]pyrido[3,4-b]pyrazin-7-yl]-N,N-dimethyl-benzenamine; N,N-dimethyl-4-[5-[(2S)-2-morpholinylmethoxy]pyrido[3,4-b]pyrazin-7-yl]-benzenamine; (2S)-2-[[[7-[4-(dimethylamino)phenyl]pyrido[3,4-b]pyrazin-5-yl]oxy]methyl]-4-morpholinecarboxylic acid 1,1-dimethylethyl ester; 3-chloro-1-[(2S)-2-[[[7-[4-(4-morpholinyl)phenyl]pyrido[3,4-b]pyrazin-5-yl]oxy]methyl]-4-morpholinyl]-1-propanone; 5-[(2S)-2-morpholinylmethoxy]-7-[4-(4-morpholinyl)phenyl]-pyrido[3,4-b]pyrazine; (2S)-2-[[[7-[4-(4-morpholinyl)phenyl]pyrido[3,4-b]pyrazin-5-yl]oxy]methyl]-4-morpholinecarboxylic acid 1,1-dimethylethyl ester; 4-[[7-[4-(4-morpholinyl)phenyl]pyrido[3,4-b]pyrazin-5-yl]oxy]-benzoic acid; 4-[[7-[4-(4-morpholinyl)phenyl]pyrido[3,4-b]pyrazin-5-yl]oxy]-benzoic acid methyl ester; 4-[[7-[4-(4-morpholinyl)phenyl]pyrido[3,4-b]pyrazin-5-yl]oxy]-cyclohexanone; (6S)-6-[[[7-[4-(1-hydroxy-1-methylethyl)phenyl]pyrido[3,4-b]pyrazin-5-yl]oxy]methyl]-4-methyl-3-morpholinone; α,α-dimethyl-4-[5-[[(2S)-4-methyl-5-oxo-2-morpholinyl]methoxy]pyrido[3,4-b]pyrazin-7-yl]-benzeneacetonitrile; (6S)-4-methyl-6-[[[7-[4-[1-(methylsulfonyl)-4-piperidinyl]phenyl]pyrido[3,4-b]pyrazin-5-yl]oxy]methyl]-3-morpholinone; (6S)-6-[[[7-(3,4-dimethoxyphenyl)pyrido[3,4-b]pyrazin-5-yl]oxy]methyl]-4-methyl-3-morpholinone; (6S)-4-methyl-6-[[[7-[4-(4-methyl-1-piperazinyl)phenyl]pyrido[3,4-b]pyrazin-5-yl]oxy]methyl]-3-morpholinone; (6S)-4-methyl-6-[[[7-[4-(4-morpholinyl)phenyl]pyrido[3,4-b]pyrazin-5-yl]oxy]methyl]-3-morpholinone; (2S)-2-[[[7-[4-(1-acetyl-3-azetidinyl)phenyl]pyrido[3,4-b]pyrazin-5-yl]oxy]methyl]-4-morpholinecarboxamide; 1-[(2S)-2-[[[7-[4-(1-acetyl-3-azetidinyl)phenyl]pyrido[3,4-b]pyrazin-5-yl]oxy]methyl]-4-morpholinyl]-ethanone; (2S)-2-[[[7-[4-(1-cyanocyclobutyl)phenyl]pyrido[3,4-b]pyrazin-5-yl]oxy]methyl]-4-morpholinesulfonamide; (2S)-2-[[[7-[4-(1-cyanocyclopropyl)phenyl]pyrido[3,4-b]pyrazin-5-yl]oxy]methyl]-4-morpholinesulfonamide; (2S)-2-[[[7-(3,4-dimethoxyphenyl)pyrido[3,4-b]pyrazin-5-yl]oxy]methyl]-4-morpholinesulfonamide; 7-[4-(1-methylethenyl)phenyl]-5-[[(2S)-4-(methylsulfonyl)-2-morpholinyl]methoxy]-pyrido[3,4-b]pyrazine; (4S)-4-[[[7-[4-[1-(methylsulfonyl)-4-piperidinyl]phenyl]pyrido[3,4-b]pyrazin-5-yl]oxy]methyl]-2-pyrrolidinone; (2S)-2-[[[7-(1H-indazol-5-yl)pyrido[3,4-b]pyrazin-5-yl]oxy]methyl]-4-morpholinecarboxamide; (2S)-2-[[[7-[4-(tetrahydro-2H-pyran-4-yl)phenyl]pyrido[3,4-b]pyrazin-5-yl]oxy]methyl]-4-morpholinecarboxamide; α-methyl-4-[5-[[(2S)-4-(methylsulfonyl)-2-morpholinyl]methoxy]pyrido[3,4-b]pyrazin-7-yl]-benzenemethanol; 7-[4-(4-morpholinyl)phenyl]-5-[(3R)-3-pyrrolidinyloxy]-pyrido[3,4-b]pyrazine; (4R)-4-[[[7-[4-(dimethylamino)phenyl]pyrido[3,4-b]pyrazin-5-yl]oxy]methyl]-2-pyrrolidinone; (4S)-4-[[[7-[4-(dimethylamino)-3-methylphenyl]pyrido[3,4-b]pyrazin-5-yl]oxy]methyl]-2-pyrrolidinone; (4S)-4-[[[7-[4-(4-methyl-1-piperazinyl)phenyl]pyrido[3,4-b]pyrazin-5-yl]oxy]methyl]-2-pyrrolidinone; (4S)-4-[[[7-[3-methyl-4-(4-morpholinyl)phenyl]pyrido[3,4-b]pyrazin-5-yl]oxy]methyl]-2-pyrrolidinone; (4S)-4-[[[7-[4-(4-morpholinyl)phenyl]pyrido[3,4-b]pyrazin-5-yl]oxy]methyl]-2-pyrrolidinone; (4S)-4-[[[7-[4-(dimethylamino)phenyl]pyrido[3,4-b]pyrazin-5-yl]oxy]methyl]-2-pyrrolidinone; 1-azetidinyl[(2S)-2-[[[7-[4-(dimethylamino)phenyl]pyrido[3,4-b]pyrazin-5-yl]oxy]methyl]-4-morpholinyl]-methanone; 1-[(2S)-2-[[[7-[4-[methyl(1-methylethyl)amino]phenyl]pyrido[3,4-b]pyrazin-5-yl]oxy]methyl]-4-morpholinyl]-ethanone; (2S)-2-[[[7-(3,4-dihydro-1,1-dimethyl-1H-2-benzopyran-6-yl)pyrido[3,4-b]pyrazin-5-yl]oxy]methyl]-4-morpholinecarboxamide; (2S)-2-[[[7-(1,3-dihydro-1,1-dimethyl-5-isobenzofuranyl)pyrido[3,4-b]pyrazin-5-yl]oxy]methyl]-4-morpholinecarboxamide; (2S)-2-[[[7-[4-(1-cyanocyclobutyl)phenyl]pyrido[3,4-b]pyrazin-5-yl]oxy]methyl]-4-morpholinecarboxamide; (2S)-2-[[[7-[4-(1-ethyl-1-hydroxypropyl)phenyl]pyrido[3,4-b]pyrazin-5-yl]oxy]methyl]-4-morpholinecarboxamide; (2S)-2-[[[7-[4-(1-cyanocyclopropyl)phenyl]pyrido[3,4-b]pyrazin-5-yl]oxy]methyl]-4-morpholinecarboxamide; (2S)-2-[[[7-[4-(1,1-dimethylethyl)phenyl]pyrido[3,4-b]pyrazin-5-yl]oxy]methyl]-4-morpholinecarboxamide; (2S)-2-[[[7-[4-(1-cyano-1-methylethyl)phenyl]pyrido[3,4-b]pyrazin-5-yl]oxy]methyl]-4-morpholinecarboxamide; (2S)-2-[(1S)-1-[[7-[4-(dimethylamino)phenyl]pyrido[3,4-b]pyrazin-5-yl]oxy]ethyl]-4-morpholinecarboxamide; (2S)-2-[(1R)-1-[[7-[4-(dimethylamino)phenyl]pyrido[3,4-b]pyrazin-5-yl]oxy]ethyl]-4-morpholinecarboxamide; (2S)-2-[[[7-(2,3-dihydro-1-methyl-1H-indol-5-yl)pyrido[3,4-b]pyrazin-5-yl]oxy]methyl]-4-morpholinecarboxamide; (2S)-2-[[[7-(1,2,3,4-tetrahydro-1-methyl-6-quinolinyl)pyrido[3,4-b]pyrazin-5-yl]oxy]methyl]-4-morpholinecarboxamide; (2S)-2-[[[7-[4-(dimethylamino)-3-fluorophenyl]pyrido[3,4-b]pyrazin-5-yl]oxy]methyl]-4-morpholinecarboxamide; (2S)-2-[[[7-[4-(1-methylethyl)phenyl]pyrido[3,4-b]pyrazin-5-yl]oxy]methyl]-4-morpholinecarboxamide; (2S)-2-[[[7-[3-chloro-4-(dimethylamino)phenyl]pyrido[3,4-b]pyrazin-5-yl]oxy]methyl]-4-morpholinecarboxamide; (2S)-2-[[[7-[4-(dimethylamino)-3-methylphenyl]pyrido[3,4-b]pyrazin-5-yl]oxy]methyl]-4-morpholinecarboxamide; (2S)-2-[[[7-(3,4-dimethoxyphenyl)pyrido[3,4-b]pyrazin-5-yl]oxy]methyl]-4-morpholinecarboxamide; [(2S)-2-[[[7-[4-(methylamino)phenyl]pyrido[3,4-b]pyrazin-5-yl]oxy]methyl]-4-morpholinyl][(3 S)-1-methyl-3-pyrrolidinyl]-methanone; N′-[2-[(2S)-2-[[[7-[4-(dimethylamino)phenyl]pyrido[3,4-b]pyrazin-5-yl]oxy]methyl]-4-morpholinyl]ethyl]-N,N-dimethyl-sulfamide; N′-[2-[(2S)-2-[[[7-[4-(dimethylamino)phenyl]pyrido[3,4-b]pyrazin-5-yl]oxy]methyl]-4-morpholinyl]ethyl]-N,N-dimethyl-urea; N-[2-[(2S)-2-[[[7-[4-(dimethylamino)phenyl]pyrido[3,4-b]pyrazin-5-yl]oxy]methyl]-4-morpholinyl]ethyl]-methanesulfonamide; N-[2-[(2S)-2-[[[7-[4-(dimethylamino)phenyl]pyrido[3,4-b]pyrazin-5-yl]oxy]methyl]-4-morpholinyl]ethyl]-acetamide; 2-[[2-[[(2S)-2-[[[7-[4-(dimethylamino)phenyl]pyrido[3,4-b]pyrazin-5-yl]oxy]methyl]-4-morpholinyl]sulfonyl]ethyl]methylamino]-acetonitrile; (3S)-1-[2-[[(2S)-2-[[[7-[4-(dimethylamino)phenyl]pyrido[3,4-b]pyrazin-5-yl]oxy]methyl]-4-morpholinyl]sulfonyl]ethyl]-3-pyrrolidinol; (3R)-1-[2-[[(2S)-2-[[[7-[4-(dimethylamino)phenyl]pyrido[3,4-b]pyrazin-5-yl]oxy]methyl]-4-morpholinyl]sulfonyl]ethyl]-3-pyrrolidinol; N,N-dimethyl-4-[5-[[(2S)-4-[[2-(2-methyl-1H-imidazol-1-yl)ethyl]sulfonyl]-2-morpholinyl]methoxy]pyrido[3,4-b]pyrazin-7-yl]-benzenamine; tetrahydro-4-[4-[5-[[(2S)-4-(methylsulfonyl)-2-morpholinyl]methoxy]pyrido[3,4-b]pyrazin-7-yl]phenyl]-2H-pyran-4-ol; N-[1-methyl-1-[4-[5-[[(2S)-4-(methylsulfonyl)-2-morpholinyl]methoxy]pyrido[3,4-b]pyrazin-7-yl]phenyl]ethyl]-acetamide; (2S)-2-[[[7-[4-(1-hydroxycyclobutyl)phenyl]pyrido[3,4-b]pyrazin-5-yl]oxy]methyl]-4-morpholinesulfonamide; 7-(3,4-dihydro-1,1-dimethyl-1H-2-benzopyran-6-yl)-5-[[(2S)-4-(methylsulfonyl)-2-morpholinyl]methoxy]-pyrido[3,4-b]pyrazine; 1-[(2S)-2-[[[7-(3,4-dihydro-1,1-dimethyl-1H-2-benzopyran-6-yl)pyrido[3,4-b]pyrazin-5-yl]oxy]methyl]-4-morpholinyl]-ethanone; 7-(1,3-dihydro-1,1-dimethyl-5-isobenzofuranyl)-5-[[(2S)-4-(methylsulfonyl)-2-morpholinyl]methoxy]-pyrido[3,4-b]pyrazine; 1-[(2S)-2-[[[7-(1,3-dihydro-1,1-dimethyl-5-isobenzofuranyl)pyrido[3,4-b]pyrazin-5-yl]oxy]methyl]-4-morpholinyl]-ethanone; 1-[4-[5-[[(2S)-4-acetyl-2-morpholinyl]methoxy]pyrido[3,4-b]pyrazin-7-yl]phenyl]-cyclobutanecarbonitrile; (2S)-2-[[[7-[4-(1-ethyl-1-hydroxypropyl)phenyl]pyrido[3,4-b]pyrazin-5-yl]oxy]methyl]-4-morpholinesulfonamide; 1-[4-[5-[[(2S)-4-acetyl-2-morpholinyl]methoxy]pyrido[3,4-b]pyrazin-7-yl]phenyl]-cyclopropanecarbonitrile; (2S)-2-[[[7-[4-(tetrahydro-2H-pyran-4-yl)phenyl]pyrido[3,4-b]pyrazin-5-yl]oxy]methyl]-4-morpholinesulfonamide; (2S)-2-[[[7-[4-(1,1-dimethylethyl)phenyl]pyrido[3,4-b]pyrazin-5-yl]oxy]methyl]-4-morpholinesulfonamide; 7-[4-(1,1-dimethylethyl)phenyl]-5-[[(2S)-4-(methylsulfonyl)-2-morpholinyl]methoxy]-pyrido[3,4-b]pyrazine; 1-[(2S)-2-[[[7-[4-(1,1-dimethylethyl)phenyl]pyrido[3,4-b]pyrazin-5-yl]oxy]methyl]-4-morpholinyl]-ethanone; (2S)-2-[[[7-[4-(1-cyano-1-methylethyl)phenyl]pyrido[3,4-b]pyrazin-5-yl]oxy]methyl]-N-methyl-4-morpholinesulfonamide; (2S)-2-[[[7-[4-(1-cyano-1-methylethyl)phenyl]pyrido[3,4-b]pyrazin-5-yl]oxy]methyl]-4-morpholinesulfonamide; (2S)-2-[(1 S)-1-[[7-[4-(dimethylamino)phenyl]pyrido[3,4-b]pyrazin-5-yl]oxy]ethyl]-4-morpholinesulfonamide; (2S)-2-[(1R)-1-[[7-[4-(dimethylamino)phenyl]pyrido[3,4-b]pyrazin-5-yl]oxy]ethyl]-4-morpholinesulfonamide; N,N-dimethyl-4-[5-[(1S)-1-[(2S)-4-(methylsulfonyl)-2-morpholinyl]ethoxy]pyrido[3,4-b]pyrazin-7-yl]-benzenamine; (2S)-2-[(1 S)-1-[[7-[4-(dimethylamino)phenyl]pyrido[3,4-b]pyrazin-5-yl]oxy]ethyl]-N-methyl-4-morpholinecarboxamide; 1-[(2S)-2-[(1S)-1-[[7-[4-(dimethylamino)phenyl]pyrido[3,4-b]pyrazin-5-yl]oxy]ethyl]-4-morpholinyl]-ethanone; N,N-dimethyl-4-[5-[(1R)-1-[(2S)-4-(methylsulfonyl)-2-morpholinyl]ethoxy]pyrido[3,4-b]pyrazin-7-yl]-benzenamine; (2S)-2-[(1R)-1-[[7-[4-(dimethylamino)phenyl]pyrido[3,4-b]pyrazin-5-yl]oxy]ethyl]-N-methyl-4-morpholinecarboxamide; 1-[(2S)-2-[(1R)-1-[[7-[4-(dimethylamino)phenyl]pyrido[3,4-b]pyrazin-5-yl]oxy]ethyl]-4-morpholinyl]-ethanone; (2S)-2-[[[7-(2,3-dihydro-1-methyl-1H-indol-5-yl)pyrido[3,4-b]pyrazin-5-yl]oxy]methyl]-4-morpholinesulfonamide; (2S)-2-[[[7-[4-(dimethylamino)phenyl]pyrido[3,4-b]pyrazin-5-yl]oxy]methyl]-N-methyl-4-morpholinesulfonamide; (2S)-2-[[[7-(1,2,3,4-tetrahydro-1-methyl-6-quinolinyl)pyrido[3,4-b]pyrazin-5-yl]oxy]methyl]-4-morpholinesulfonamide; 5-[[(2S)-4-(methylsulfonyl)-2-morpholinyl]methoxy]-7-(1,2,3,4-tetrahydro-1-methyl-6-quinolinyl)-pyrido[3,4-b]pyrazine; 1-[(2S)-2-[[[7-[4-(dimethylamino)-3-methylphenyl]pyrido[3,4-b]pyrazin-5-yl]oxy]methyl]-4-morpholinyl]-1-propanone; (2S)-1-[(2S)-2-[[[7-[4-(dimethylamino)-3-methylphenyl]pyrido[3,4-b]pyrazin-5-yl]oxy]methyl]-4-morpholinyl]-2-hydroxy-1-propanone; (2R)-1-[(2S)-2-[[[7-[4-(dimethylamino)-3-methylphenyl]pyrido[3,4-b]pyrazin-5-yl]oxy]methyl]-4-morpholinyl]-2-hydroxy-1-propanone; 1-[(2S)-2-[[[7-[4-(dimethylamino)phenyl]pyrido[3,4-b]pyrazin-5-yl]oxy]methyl]-4-morpholinyl]-1-propanone; (2S)-1-[(2S)-2-[[[7-[4-(dimethylamino)phenyl]pyrido[3,4-b]pyrazin-5-yl]oxy]methyl]-4-morpholinyl]-2-hydroxy-1-propanone; (2R)-1-[(2S)-2-[[[7-[4-(dimethylamino)phenyl]pyrido[3,4-b]pyrazin-5-yl]oxy]methyl]-4-morpholinyl]-2-hydroxy-1-propanone; 2-hydroxy-1-[(2S)-2-[[[7-[4-(1-methylethyl)phenyl]pyrido[3,4-b]pyrazin-5-yl]oxy]methyl]-4-morpholinyl]-ethanone; (2S)-2-[[[7-[4-(1-methylethyl)phenyl]pyrido[3,4-b]pyrazin-5-yl]oxy]methyl]-4-morpholinesulfonamide; (2S)-2-[[[7-[4-(dimethylamino)-3-fluorophenyl]pyrido[3,4-b]pyrazin-5-yl]oxy]methyl]-N-methyl-4-morpholinecarboxamide; 7-[4-(1-methylethyl)phenyl]-5-[[(2S)-4-(methylsulfonyl)-2-morpholinyl]methoxy]-pyrido[3,4-b]pyrazine; (2S)—N-methyl-2-[[[7-[4-(1-methylethyl)phenyl]pyrido[3,4-b]pyrazin-5-yl]oxy]methyl]-4-morpholinecarboxamide; 1-[(2S)-2-[[[7-[4-(dimethylamino)phenyl]pyrido[3,4-b]pyrazin-5-yl]oxy]methyl]-4-morpholinyl]-2-hydroxy-ethanone; 1-[(2S)-2-[[[7-[4-(dimethylamino)phenyl]pyrido[3,4-b]pyrazin-5-yl]oxy]methyl]-4-morpholinyl]-2,2-difluoro-1-propanone; (2S)-2-[[[7-[3-chloro-4-(dimethylamino)phenyl]pyrido[3,4-b]pyrazin-5-yl]oxy]methyl]-N-methyl-4-morpholinecarboxamide; (2S)-2-[[[7-[4-(dimethylamino)-3-methylphenyl]pyrido[3,4-b]pyrazin-5-yl]oxy]methyl]-N-methyl-4-morpholinecarboxamide; (2S)-2-[[[7-[4-(dimethylamino)phenyl]pyrido[3,4-b]pyrazin-5-yl]oxy]methyl]-N,N-dimethyl-4-morpholinecarboxamide; (2S)-2-[[[7-(3,4-dimethoxyphenyl)pyrido[3,4-b]pyrazin-5-yl]oxy]methyl]-N-methyl-4-morpholinecarboxamide; (2S)-2-[[[7-[4-(dimethylamino)phenyl]pyrido[3,4-b]pyrazin-5-yl]oxy]methyl]-N-methyl-4-morpholinecarboxamide; (2S)-2-[[[7-[4-(dimethylamino)phenyl]pyrido[3,4-b]pyrazin-5-yl]oxy]methyl]-4-morpholinecarboxamide; (2S)-2-[[[7-[4-(dimethylamino)phenyl]pyrido[3,4-b]pyrazin-5-yl]oxy]methyl]-N-methyl-4-morpholineethanesulfonamide; (2S)-2-[[[7-[4-(dimethylamino)phenyl]pyrido[3,4-b]pyrazin-5-yl]oxy]methyl]-N-methyl-4-morpholinepropanamide; (2S)-2-[[[7-[4-(dimethylamino)phenyl]pyrido[3,4-b]pyrazin-5-yl]oxy]methyl]-N-methyl-4-morpholineacetamide; 2-[[(2S)-2-[[[7-[4-(dimethylamino)phenyl]pyrido[3,4-b]pyrazin-5-yl]oxy]methyl]-4-morpholinyl]sulfonyl]-N-methyl-acetamide; cyclopentyl[(2S)-2-[[[7-[4-(dimethylamino)phenyl]pyrido[3,4-b]pyrazin-5-yl]oxy]methyl]-4-morpholinyl]-methanone; 1-[[(2S)-2-[[[7-[4-(dimethylamino)phenyl]pyrido[3,4-b]pyrazin-5-yl]oxy]methyl]-4-morpholinyl]carbonyl]-cyclopropanecarbonitrile; (2,2-difluorocyclopropyl)[(2S)-2-[[[7-[4-(dimethylamino)phenyl]pyrido[3,4-b]pyrazin-5-yl]oxy]methyl]-4-morpholinyl]-methanone; [(2S)-2-[[[7-[4-(dimethylamino)phenyl]pyrido[3,4-b]pyrazin-5-yl]oxy]methyl]-4-morpholinyl][(3 S)-tetrahydro-3-furanyl]-methanone; N,N-dimethyl-4-[5-[[(2S)-4-(methylsulfonyl)-2-morpholinyl]methoxy]pyrido[3,4-b]pyrazin-7-yl]-benzenamine; 4-[5-[[(2S)-4-[[(3-fluorophenyl)methyl]sulfonyl]-2-morpholinyl]methoxy]pyrido[3,4-b]pyrazin-7-yl]-N,N-dimethyl-benzenamine; N,N-dimethyl-4-[5-[[(2S)-4-(3-pyridinylsulfonyl)-2-morpholinyl]methoxy]pyrido[3,4-b]pyrazin-7-yl]-benzenamine; 4-[5-[[(2S)-4-(cyclohexylsulfonyl)-2-morpholinyl]methoxy]pyrido[3,4-b]pyrazin-7-yl]-N,N-dimethyl-benzenamine; 4-[5-[[(2S)-4-(cyclopropylsulfonyl)-2-morpholinyl]methoxy]pyrido[3,4-b]pyrazin-7-yl]-N,N-dimethyl-benzenamine; N,N-dimethyl-4-[5-[[(2S)-4-[(1-methylethyl)sulfonyl]-2-morpholinyl]methoxy]pyrido[3,4-b]pyrazin-7-yl]-benzenamine; 4-[5-[[(2S)-4-(ethylsulfonyl)-2-morpholinyl]methoxy]pyrido[3,4-b]pyrazin-7-yl]-N,N-dimethyl-benzenamine; [(2S)-2-[[[7-[4-(dimethylamino)phenyl]pyrido[3,4-b]pyrazin-5-yl]oxy]methyl]-4-morpholinyl](1-methyl-1H-pyrazol-4-yl)-methanone; [(2S)-2-[[[7-[4-(dimethylamino)phenyl]pyrido[3,4-b]pyrazin-5-yl]oxy]methyl]-4-morpholinyl]-1H-imidazol-2-yl-methanone; 1-[(2S)-2-[[[7-[4-(dimethylamino)phenyl]pyrido[3,4-b]pyrazin-5-yl]oxy]methyl]-4-morpholinyl]-3-(1H-imidazol-1-yl)-1-propanone; 1-[(2S)-2-[[[7-[4-(dimethylamino)phenyl]pyrido[3,4-b]pyrazin-5-yl]oxy]methyl]-4-morpholinyl]-2-(1H-imidazol-5-yl)-ethanone; 5-[[(2S)-4-(cyclopropylsulfonyl)-2-morpholinyl]methoxy]-7-[4-(4-morpholinyl)phenyl]-pyrido[3,4-b]pyrazine; 5-[[(2S)-4-[(1-methylethyl)sulfonyl]-2-morpholinyl]methoxy]-7-[4-(4-morpholinyl)phenyl]-pyrido[3,4-b]pyrazine; 2S)-2-[[[7-[4-(dimethylamino)phenyl]pyrido[3,4-b]pyrazin-5-yl]oxy]methyl]-δ-oxo-4-morpholinepentanenitrile; 1-[(2S)-2-[[[7-[4-(dimethylamino)phenyl]pyrido[3,4-b]pyrazin-5-yl]oxy]methyl]-4-morpholinyl]-2-(3-pyridinyl)-ethanone; 1-[(2S)-2-[[[7-[4-(dimethylamino)phenyl]pyrido[3,4-b]pyrazin-5-yl]oxy]methyl]-4-morpholinyl]-3-(3-pyridinyl)-1-propanone; [(2S)-2-[[[7-[4-(dimethylamino)phenyl]pyrido[3,4-b]pyrazin-5-yl]oxy]methyl]-4-morpholinyl]-3-pyridinyl-methanone; (2S)-2-[[[7-[4-(dimethylamino)phenyl]pyrido[3,4-b]pyrazin-5-yl]oxy]methyl]-γ-oxo-4-morpholinebutanenitrile; (2S)-2-[[[7-[4-(dimethylamino)phenyl]pyrido[3,4-b]pyrazin-5-yl]oxy]methyl]-β-oxo-4-morpholinepropanenitrile; [(2S)-2-[[[7-[4-(dimethylamino)phenyl]pyrido[3,4-b]pyrazin-5-yl]oxy]methyl]-4-morpholinyl](cis-4-hydroxycyclohexyl)-methanone; [(2S)-2-[[[7-[4-(dimethylamino)phenyl]pyrido[3,4-b]pyrazin-5-yl]oxy]methyl]-4-morpholinyl](trans-4-hydroxycyclohexyl)-methanone; [(2S)-2-[[[7-[4-(dimethylamino)phenyl]pyrido[3,4-b]pyrazin-5-yl]oxy]methyl]-4-morpholinyl](tetrahydro-2H-pyran-4-yl)-methanone; [(2S)-2-[[[7-[4-(dimethylamino)phenyl]pyrido[3,4-b]pyrazin-5-yl]oxy]methyl]-4-morpholinyl][(3R)-tetrahydro-3-furanyl]-methanone; (3,3-difluorocyclobutyl)[(2S)-2-[[[7-[4-(dimethylamino)phenyl]pyrido[3,4-b]pyrazin-5-yl]oxy]methyl]-4-morpholinyl]-methanone; 2-cyclopropyl-1-[(2S)-2-[[[7-[4-(dimethylamino)phenyl]pyrido[3,4-b]pyrazin-5-yl]oxy]methyl]-4-morpholinyl]-ethanone; cyclopropyl[(2S)-2-[[[7-[4-(dimethylamino)phenyl]pyrido[3,4-b]pyrazin-5-yl]oxy]methyl]-4-morpholinyl]-methanone; 1-[(2S)-2-[[[7-[4-(dimethylamino)phenyl]pyrido[3,4-b]pyrazin-5-yl]oxy]methyl]-4-morpholinyl]-2-methyl-1-propanone; 1-[(2S)-2-[[[7-[4-(dimethylamino)phenyl]pyrido[3,4-b]pyrazin-5-yl]oxy]methyl]-4-morpholinyl]-ethanone; 5-[[(2S)-4-[(2-methoxyethyl)sulfonyl]-2-morpholinyl]methoxy]-7-[4-(4-morpholinyl)phenyl]-pyrido[3,4-b]pyrazine; N,N-dimethyl-2-[[(2S)-2-[[[7-[4-(4-morpholinyl)phenyl]pyrido[3,4-b]pyrazin-5-yl]oxy]methyl]-4-morpholinyl]sulfonyl]-ethanamine; (2S)-2-[[[7-[4-(4-methyl-1-piperazinyl)phenyl]pyrido[3,4-b]pyrazin-5-yl]oxy]methyl]-4-morpholinesulfonamide; (2S)-2-[[[7-[4-(4-morpholinyl)phenyl]pyrido[3,4-b]pyrazin-5-yl]oxy]methyl]-4-morpholinecarboxamide; (2S)-2-[[[7-[4-(4-morpholinyl)phenyl]pyrido[3,4-b]pyrazin-5-yl]oxy]methyl]-4-morpholinesulfonamide; 2,2-difluoro-1-[(2S)-2-[[[7-[4-(4-morpholinyl)phenyl]pyrido[3,4-b]pyrazin-5-yl]oxy]methyl]-4-morpholinyl]-ethanone; 3-(dimethylamino)-1-[(2S)-2-[[[7-[4-(4-morpholinyl)phenyl]pyrido[3,4-b]pyrazin-5-yl]oxy]methyl]-4-morpholinyl]-1-propanone; 2-[2-methoxy-4-[5-[[(2S)-4-(methylsulfonyl)-2-morpholinyl]methoxy]pyrido[3,4-b]pyrazin-7-yl]phenoxy]-N,N-dimethyl-ethanamine; 7-[4-[1-(2-methoxyethyl)-4-piperidinyl]phenyl]-5-[[(2S)-4-(methylsulfonyl)-2-morpholinyl]methoxy]-pyrido[3,4-b]pyrazine; 4-[4-[5-[[(2S)-4-(methylsulfonyl)-2-morpholinyl]methoxy]pyrido[3,4-b]pyrazin-7-yl]phenyl]-1-piperidineethanol; 5-[[(2S)-4-(methylsulfonyl)-2-morpholinyl]methoxy]-7-[4-[1-(methylsulfonyl)-4-piperidinyl]phenyl]-pyrido[3,4-b]pyrazine; 1-[(2S)-2-[[[7-(4-fluoro-3,4-dihydro-1,1-dimethyl-1H-2-benzopyran-6-yl)pyrido[3,4-b]pyrazin-5-yl]oxy]methyl]-4-morpholinyl]-ethanone; 7-(4-fluoro-3,4-dihydro-1,1-dimethyl-1H-2-benzopyran-6-yl)-5-[[(2S)-4-(methylsulfonyl)-2-morpholinyl]methoxy]-pyrido[3,4-b]pyrazine; 1-[3-methyl-3-[4-[5-[[(2S)-4-(methylsulfonyl)-2-morpholinyl]methoxy]pyrido[3,4-b]pyrazin-7-yl]phenyl]-1-azetidinyl]-ethanone; α,α-dimethyl-4-[5-[[(2S)-4-(methylsulfonyl)-2-morpholinyl]methoxy]pyrido[3,4-b]pyrazin-7-yl]-benzeneacetic acid; 7-[4-(3-methyl-3-oxetanyl)phenyl]-5-[[(2S)-4-(methylsulfonyl)-2-morpholinyl]methoxy]-pyrido[3,4-b]pyrazine; (2S)-2-[[[7-[4-(1-hydroxycyclobutyl)phenyl]pyrido[3,4-b]pyrazin-5-yl]oxy]methyl]-4-morpholinecarboxamide; 1-[(2S)-2-[[[7-[4-(1-hydroxycyclobutyl)phenyl]pyrido[3,4-b]pyrazin-5-yl]oxy]methyl]-4-morpholinyl]-ethanone; 1-[4-[5-[[(2S)-4-(methylsulfonyl)-2-morpholinyl]methoxy]pyrido[3,4-b]pyrazin-7-yl]phenyl]-cyclobutanol; 1-[(2S)-2-[[[7-[4-(1-amino-2-methylpropyl)phenyl]pyrido[3,4-b]pyrazin-5-yl]oxy]methyl]-4-morpholinyl]-ethanone; β,β-dimethyl-4-[5-[[(2S)-4-(methylsulfonyl)-2-morpholinyl]methoxy]pyrido[3,4-b]pyrazin-7-yl]-benzeneethanol; α-(1-methylethyl)-4-[5-[[(2S)-4-(methylsulfonyl)-2-morpholinyl]methoxy]pyrido[3,4-b]pyrazin-7-yl]-benzenemethanol; 1-[(2S)-2-[[[7-[4-(1-hydroxy-2-methylpropyl)phenyl]pyrido[3,4-b]pyrazin-5-yl]oxy]methyl]-4-morpholinyl]-ethanone; 1-[(2S)-2-[[[7-[4-(1-hydroxy-2,2-dimethylpropyl)phenyl]pyrido[3,4-b]pyrazin-5-yl]oxy]methyl]-4-morpholinyl]-ethanone; α-(1,1-dimethylethyl)-4-[5-[[(2S)-4-(methylsulfonyl)-2-morpholinyl]methoxy]pyrido[3,4-b]pyrazin-7-yl]-benzenemethanol; 1-[4-[5-[[(2S)-4-(methylsulfonyl)-2-morpholinyl]methoxy]pyrido[3,4-b]pyrazin-7-yl]phenyl]-cyclobutanecarbonitrile; 1-[(2S)-2-[[[7-[4-(1-methoxyethyl)phenyl]pyrido[3,4-b]pyrazin-5-yl]oxy]methyl]-4-morpholinyl]-ethanone; α,α-diethyl-4-[5-[[(2S)-4-(methylsulfonyl)-2-morpholinyl]methoxy]pyrido[3,4-b]pyrazin-7-yl]-benzenemethanol; 1-[(2S)-2-[[[7-[4-[2-hydroxy-1-(hydroxymethyl)-1-methylethyl]phenyl]pyrido[3,4-b]pyrazin-5-yl]oxy]methyl]-4-morpholinyl]-ethanone; 1-[4-[5-[[(2S)-4-(methylsulfonyl)-2-morpholinyl]methoxy]pyrido[3,4-b]pyrazin-7-yl]phenyl]-cyclopropanecarbonitrile; α,α-dimethyl-4-[5-[[(2S)-4-(methylsulfonyl)-2-morpholinyl]methoxy]pyrido[3,4-b]pyrazin-7-yl]-benzeneethanol; N,N-dimethyl-4-[5-[[(2S)-4-(methylsulfonyl)-2-morpholinyl]methoxy]pyrido[3,4-b]pyrazin-7-yl]-benzeneethanamine; 1-[(2S)-2-[[[7-[4-[1-(dimethylamino)ethyl]phenyl]pyrido[3,4-b]pyrazin-5-yl]oxy]methyl]-4-morpholinyl]-ethanone; 1-[(2S)-2-[[[7-[4-(1-ethyl-1-hydroxypropyl)phenyl]pyrido[3,4-b]pyrazin-5-yl]oxy]methyl]-4-morpholinyl]-ethanone; 1-[(2S)-2-[[[7-[4-(1-methoxy-1-methylethyl)phenyl]pyrido[3,4-b]pyrazin-5-yl]oxy]methyl]-4-morpholinyl]-ethanone; 7-[4-(1-methoxy-1-methylethyl)phenyl]-5-[[(2S)-4-(methylsulfonyl)-2-morpholinyl]methoxy]-pyrido[3,4-b]pyrazine; 1-[(2S)-2-[[[7-[4-(2-amino-1,1-dimethylethyl)phenyl]pyrido[3,4-b]pyrazin-5-yl]oxy]methyl]-4-morpholinyl]-ethanone; (2S)-2-[[[7-[4-(1-hydroxy-1-methylethyl)phenyl]pyrido[3,4-b]pyrazin-5-yl]oxy]methyl]-4-morpholinecarboxamide; 1-[(2S)-2-[[[7-[4-(1-hydroxy-1-methylethyl)phenyl]pyrido[3,4-b]pyrazin-5-yl]oxy]methyl]-4-morpholinyl]-ethanone; (2S)-2-[[[7-[4-(1-hydroxy-1-methylethyl)phenyl]pyrido[3,4-b]pyrazin-5-yl]oxy]methyl]-N-methyl-4-morpholinecarboxamide; (2S)-2-[[[7-[4-(1-hydroxy-1-methylethyl)phenyl]pyrido[3,4-b]pyrazin-5-yl]oxy]methyl]-4-morpholinesulfonamide; (2S)-2-[[[7-[4-(1-hydroxy-1-methylethyl)phenyl]pyrido[3,4-b]pyrazin-5-yl]oxy]methyl]-N-methyl-4-morpholinesulfonamide; N-methyl-N-[[4-[5-[[(2S)-4-(methylsulfonyl)-2-morpholinyl]methoxy]pyrido[3,4-b]pyrazin-7-yl]phenyl]methyl]-acetamide; 5-[[(2S)-4-(methylsulfonyl)-2-morpholinyl]methoxy]-7-[4-(4-morpholinylmethyl)phenyl]-pyrido[3,4-b]pyrazine; 1-[(2S)-2-[[[7-[4-[(dimethylamino)methyl]phenyl]pyrido[3,4-b]pyrazin-5-yl]oxy]methyl]-4-morpholinyl]-ethanone; (2S)-2-[[[7-[4-(1-cyano-1-methylethyl)phenyl]pyrido[3,4-b]pyrazin-5-yl]oxy]methyl]-N-methyl-4-morpholinecarboxamide; 4-[5-[[(2S)-4-acetyl-2-morpholinyl]methoxy]pyrido[3,4-b]pyrazin-7-yl]-α,α-dimethyl-benzeneacetonitrile; 1-[(2S)-2-[[[7-[4-(1-amino-1-methylethyl)phenyl]pyrido[3,4-b]pyrazin-5-yl]oxy]methyl]-4-morpholinyl]-ethanone; α,α-dimethyl-4-[5-[[(2S)-4-(methylsulfonyl)-2-morpholinyl]methoxy]pyrido[3,4-b]pyrazin-7-yl]-benzenemethanamine; α,α-dimethyl-4-[5-[[(2S)-4-(methylsulfonyl)-2-morpholinyl]methoxy]pyrido[3,4-b]pyrazin-7-yl]-benzeneacetamide; 7-[4-(1,1-difluoroethyl)phenyl]-5-[[(2S)-4-(methylsulfonyl)-2-morpholinyl]methoxy]-pyrido[3,4-b]pyrazine; (2S)-2-[[[7-(4-ethylphenyl)pyrido[3,4-b]pyrazin-5-yl]oxy]methyl]-N-methyl-4-morpholinecarboxamide; (2S)-2-[[[7-[4-(dimethylamino)-3-methylphenyl]pyrido[3,4-b]pyrazin-5-yl]oxy]methyl]-N-methyl-4-morpholinesulfonamide; (2S)-2-[[[7-(3,4-dimethoxyphenyl)pyrido[3,4-b]pyrazin-5-yl]oxy]methyl]-N-methyl-4-morpholinesulfonamide; α,α-dimethyl-4-[5-[[(2S)-4-(methylsulfonyl)-2-morpholinyl]methoxy]pyrido[3,4-b]pyrazin-7-yl]-benzenemethanol; N,N-dimethyl-5-[5-[[(2S)-4-(methylsulfonyl)-2-morpholinyl]methoxy]pyrido[3,4-b]pyrazin-7-yl]-2-pyridinamine; 7-(2,3-dihydro-1-methyl-1H-indol-5-yl)-5-[[(2S)-4-(methylsulfonyl)-2-morpholinyl]methoxy]-pyrido[3,4-b]pyrazine; α,α-dimethyl-4-[5-[[(2S)-4-(methylsulfonyl)-2-morpholinyl]methoxy]pyrido[3,4-b]pyrazin-7-yl]-benzeneacetonitrile; (2S)-2-[[[7-(1H-indazol-5-yl)pyrido[3,4-b]pyrazin-5-yl]oxy]methyl]-N-methyl-4-morpholinecarboxamide; 7-(1H-indazol-5-yl)-5-[[(2S)-4-(methylsulfonyl)-2-morpholinyl]methoxy]-pyrido[3,4-b]pyrazine; 1-[(2S)-2-[[[7-(2,3-dihydro-1-methyl-1H-indol-5-yl)pyrido[3,4-b]pyrazin-5-yl]oxy]methyl]-4-morpholinyl]-ethanone; (2S)-2-[[[7-[4-(dimethylamino)-3-methylphenyl]pyrido[3,4-b]pyrazin-5-yl]oxy]methyl]-4-morpholinesulfonamide; 1-[(2S)-2-[[[7-[3-methyl-4-(4-methyl-1-piperazinyl)phenyl]pyrido[3,4-b]pyrazin-5-yl]oxy]methyl]-4-morpholinyl]-ethanone; 1-[(2S)-2-[[[7-(1,2,3,4-tetrahydro-1-methyl-6-quinolinyl)pyrido[3,4-b]pyrazin-5-yl]oxy]methyl]-4-morpholinyl]-ethanone; 1-[(2S)-2-[[[7-[3-methyl-4-(1-piperazinyl)phenyl]pyrido[3,4-b]pyrazin-5-yl]oxy]methyl]-4-morpholinyl]-ethanone; (2S)-2-[[[7-[4-(dimethylamino)phenyl]pyrido[3,4-b]pyrazin-5-yl]oxy]methyl]-4-morpholinesulfonamide; 1-[(2S)-2-[[[7-[6-(dimethylamino)-5-methyl-3-pyridinyl]pyrido[3,4-b]pyrazin-5-yl]oxy]methyl]-4-morpholinyl]-ethanone; 5-[5-[[(2S)-4-acetyl-2-morpholinyl]methoxy]pyrido[3,4-b]pyrazin-7-yl]-1,3-dihydro-1-methyl-2H-indol-2-one; 1-[(2S)-2-[[[7-[4-(4-piperidinyl)phenyl]pyrido[3,4-b]pyrazin-5-yl]oxy]methyl]-4-morpholinyl]-ethanone; 1-[(2S)-2-[[[7-[4-(dimethylamino)-3,5-dimethylphenyl]pyrido[3,4-b]pyrazin-5-yl]oxy]methyl]-4-morpholinyl]-ethanone; 1-[(2S)-2-[[[7-[4-(1-methylethyl)phenyl]pyrido[3,4-b]pyrazin-5-yl]oxy]methyl]-4-morpholinyl]-ethanone; 1-[(2S)-2-[[[7-[4-(dimethylamino)-3-methylphenyl]pyrido[3,4-b]pyrazin-5-yl]oxy]methyl]-4-morpholinyl]-ethanone; 1-[(2S)-2-[[[7-[4-methoxy-3-(1-methylethoxy)phenyl]pyrido[3,4-b]pyrazin-5-yl]oxy]methyl]-4-morpholinyl]-ethanone; 1-[(2S)-2-[[[7-(3,4-dihydro-4-methyl-2H-1,4-benzoxazin-7-yl)pyrido[3,4-b]pyrazin-5-yl]oxy]methyl]-4-morpholinyl]-ethanone; 1-[(2S)-2-[[[7-[3-methoxy-4-(1-methylethoxy)phenyl]pyrido[3,4-b]pyrazin-5-yl]oxy]methyl]-4-morpholinyl]-ethanone; 1-[(2S)-2-[[[7-[3-(dimethylamino)phenyl]pyrido[3,4-b]pyrazin-5-yl]oxy]methyl]-4-morpholinyl]-ethanone; 1-[(2S)-2-[[[7-(3-methoxyphenyl)pyrido[3,4-b]pyrazin-5-yl]oxy]methyl]-4-morpholinyl]-ethanone; 1-[(2S)-2-[[[7-[4-[(1-methylethyl)amino]phenyl]pyrido[3,4-b]pyrazin-5-yl]oxy]methyl]-4-morpholinyl]-ethanone; 1-[(2S)-2-[[[7-[3-chloro-4-(dimethylamino)phenyl]pyrido[3,4-b]pyrazin-5-yl]oxy]methyl]-4-morpholinyl]-ethanone; 1-[(2S)-2-[[[7-[4-(dimethylamino)-3-fluorophenyl]pyrido[3,4-b]pyrazin-5-yl]oxy]methyl]-4-morpholinyl]-ethanone; 1-[(2S)-2-[[[7-(3,5-dimethoxyphenyl)pyrido[3,4-b]pyrazin-5-yl]oxy]methyl]-4-morpholinyl]-ethanone; N,N,2-trimethyl-4-[5-[[(2S)-4-(methylsulfonyl)-2-morpholinyl]methoxy]pyrido[3,4-b]pyrazin-7-yl]-benzenamine; 1-[(2S)-2-[[[7-(3,4-dimethoxyphenyl)pyrido[3,4-b]pyrazin-5-yl]oxy]methyl]-4-morpholinyl]-ethanone; 1-[(2S)-2-[[[7-[4-(diethylamino)phenyl]pyrido[3,4-b]pyrazin-5-yl]oxy]methyl]-4-morpholinyl]-ethanone; N,N-dimethyl-2-[3-[5-[[(2S)-4-(methylsulfonyl)-2-morpholinyl]methoxy]pyrido[3,4-b]pyrazin-7-yl]phenoxy]-ethanamine; N-methyl-2-[4-[5-[[(2S)-4-(methylsulfonyl)-2-morpholinyl]methoxy]pyrido[3,4-b]pyrazin-7-yl]phenoxy]-acetamide; 1-[4-[4-[5-[[(2S)-4-(methylsulfonyl)-2-morpholinyl]methoxy]pyrido[3,4-b]pyrazin-7-yl]phenyl]-1-piperazinyl]-ethanone; 5-[[(2S)-4-(methylsulfonyl)-2-morpholinyl]methoxy]-7-(3,4,5-trimethoxyphenyl)-pyrido[3,4-b]pyrazine; 7-(1-methyl-1H-indol-6-yl)-5-[[(2S)-4-(methylsulfonyl)-2-morpholinyl]methoxy]-pyrido[3,4-b]pyrazine; N,N-diethyl-4-[5-[[(2S)-4-(methylsulfonyl)-2-morpholinyl]methoxy]pyrido[3,4-b]pyrazin-7-yl]-benzenamine; 1-[(2S)-2-[[[7-[4-[(2-methoxyethyl)amino]phenyl]pyrido[3,4-b]pyrazin-5-yl]oxy]methyl]-4-morpholinyl]-ethanone; 5-[[(2S)-4-(methylsulfonyl)-2-morpholinyl]methoxy]-7-(6-quinolinyl)-pyrido[3,4-b]pyrazine; 7-(1,3-benzodioxol-5-yl)-5-[[(2S)-4-(methylsulfonyl)-2-morpholinyl]methoxy]-pyrido[3,4-b]pyrazine; 7-(2,3-dihydro-1,4-benzodioxin-6-yl)-5-[[(2S)-4-(methylsulfonyl)-2-morpholinyl]methoxy]-pyrido[3,4-b]pyrazine; 2-chloro-N,N-dimethyl-4-[5-[[(2S)-4-(methylsulfonyl)-2-morpholinyl]methoxy]pyrido[3,4-b]pyrazin-7-yl]-benzenamine; 7-(1-methyl-1H-indazol-5-yl)-5-[[(2S)-4-(methylsulfonyl)-2-morpholinyl]methoxy]-pyrido[3,4-b]pyrazine; 1-[(2S)-2-[[[7-[4-(dimethylamino)phenyl]pyrido[3,4-b]pyrazin-5-yl]oxy]methyl]-4-morpholinyl]-2,2-difluoro-ethanone; (2S)—N,N-dimethyl-2-[[[7-[4-(4-morpholinyl)phenyl]pyrido[3,4-b]pyrazin-5-yl]oxy]methyl]-4-morpholinesulfonamide; (2S)—N-methyl-2-[[[7-[4-(4-morpholinyl)phenyl]pyrido[3,4-b]pyrazin-5-yl]oxy]methyl]-4-morpholinesulfonamide; 1-[4-[4-[5-[[(2S)-4-acetyl-2-morpholinyl]methoxy]pyrido[3,4-b]pyrazin-7-yl]phenyl]-1-piperidinyl]-ethanone; 2-fluoro-N,N-dimethyl-4-[5-[[(2S)-4-(methylsulfonyl)-2-morpholinyl]methoxy]pyrido[3,4-b]pyrazin-7-yl]-benzenamine; 2-fluoro-N-methyl-4-[5-[[(2S)-4-(methylsulfonyl)-2-morpholinyl]methoxy]pyrido[3,4-b]pyrazin-7-yl]-benzenamine; [(2S)-2-[[[7-[4-(methylamino)phenyl]pyrido[3,4-b]pyrazin-5-yl]oxy]methyl]-4-morpholinyl][(2R)-tetrahydro-2-furanyl]-methanone; [(2S)-2-[[[7-[4-(methylamino)phenyl]pyrido[3,4-b]pyrazin-5-yl]oxy]methyl]-4-morpholinyl][(3 S)-tetrahydro-3-furanyl]-methanone; 1-[(2S)-2-[[[7-[4-[4-(2-hydroxyethyl)-1-piperazinyl]phenyl]pyrido[3,4-b]pyrazin-5-yl]oxy]methyl]-4-morpholinyl]-ethanone; 1-[(2S)-2-[[[7-[4-(4-methoxy-1-piperidinyl)phenyl]pyrido[3,4-b]pyrazin-5-yl]oxy]methyl]-4-morpholinyl]-ethanone; 1-[(2S)-2-[[[7-[4-(methylamino)phenyl]pyrido[3,4-b]pyrazin-5-yl]oxy]methyl]-4-morpholinyl]-ethanone; 7-(1-methyl-1H-pyrazol-4-yl)-5-[[(2S)-4-(methylsulfonyl)-2-morpholinyl]methoxy]-pyrido[3,4-b]pyrazine; 4-[5-[[(2S)-4-(methylsulfonyl)-2-morpholinyl]methoxy]pyrido[3,4-b]pyrazin-7-yl]-benzonitrile; 7-(4-fluorophenyl)-5-[[(2S)-4-(methylsulfonyl)-2-morpholinyl]methoxy]-pyrido[3,4-b]pyrazine; 7-(3,4-dimethoxyphenyl)-5-[[(2S)-4-(methylsulfonyl)-2-morpholinyl]methoxy]-pyrido[3,4-b]pyrazine; 5-[[(2S)-4-(methylsulfonyl)-2-morpholinyl]methoxy]-7-[1-(tetrahydro-2H-pyran-4-yl)-1H-pyrazol-4-yl]-Pyrido[3,4-b]pyrazine; 7-(4-methylphenyl)-5-[[(2S)-4-(methylsulfonyl)-2-morpholinyl]methoxy]-pyrido[3,4-b]pyrazine; 7-(4-chlorophenyl)-5-[[(2S)-4-(methylsulfonyl)-2-morpholinyl]methoxy]-pyrido[3,4-b]pyrazine; 7-(1-methyl-1H-indol-5-yl)-5-[[(2S)-4-(methylsulfonyl)-2-morpholinyl]methoxy]-pyrido[3,4-b]pyrazine; 7-(4-methoxyphenyl)-5-[[(2S)-4-(methylsulfonyl)-2-morpholinyl]methoxy]-pyrido[3,4-b]pyrazine; 5-[[(2S)-4-(methylsulfonyl)-2-morpholinyl]methoxy]-7-[4-(1-pyrrolidinyl)phenyl]-pyrido[3,4-b]pyrazine; N-methyl-4-[5-[[(2S)-4-(methylsulfonyl)-2-morpholinyl]methoxy]pyrido[3,4-b]pyrazin-7-yl]-benzenamine; 7-(3-fluoro-4-methoxyphenyl)-5-[[(2S)-4-(methylsulfonyl)-2-morpholinyl]methoxy]-pyrido[3,4-b]pyrazine; 7-[4-(difluoromethoxy)phenyl]-5-[[(2S)-4-(methylsulfonyl)-2-morpholinyl]methoxy]-pyrido[3,4-b]pyrazine; 1-[4-[4-[5-[[(2S)-4-(methylsulfonyl)-2-morpholinyl]methoxy]pyrido[3,4-b]pyrazin-7-yl]phenyl]-1-piperidinyl]-ethanone; 1-[(2S)-2-[[[7-[4-(dimethylamino)-3-fluorophenyl]pyrido[3,4-b]pyrazin-5-yl]oxy]methyl]-4-morpholinyl]-2,2-difluoro-ethanone; 7-[4-(4-methoxy-1-piperidinyl)phenyl]-5-[[(2S)-4-(methylsulfonyl)-2-morpholinyl]methoxy]-pyrido[3,4-b]pyrazine; (2S)-2-[[[7-[4-(1-pyrrolidinyl)phenyl]pyrido[3,4-b]pyrazin-5-yl]oxy]methyl]-4-morpholinesulfonamide; (2S)-2-[[[7-[4-(4,4-difluoro-1-piperidinyl)phenyl]pyrido[3,4-b]pyrazin-5-yl]oxy]methyl]-4-morpholinesulfonamide; (2S)-2-[[[7-[4-(1-piperidinyl)phenyl]pyrido[3,4-b]pyrazin-5-yl]oxy]methyl]-4-morpholinesulfonamide; 4-[4-[5-[[(2S)-4-(methylsulfonyl)-2-morpholinyl]methoxy]pyrido[3,4-b]pyrazin-7-yl]phenyl]-1-piperazineethanol; 5-[[(2S)-4-(methylsulfonyl)-2-morpholinyl]methoxy]-7-[4-[4-(methylsulfonyl)-1-piperazinyl]phenyl]-pyrido[3,4-b]pyrazine; 1-[(2S)-2-[[[7-[4-[4-(ethylsulfonyl)-1-piperazinyl]phenyl]pyrido[3,4-b]pyrazin-5-yl]oxy]methyl]-4-morpholinyl]-ethanone; 1-[(2S)-2-[[[7-[4-(4-morpholinyl)phenyl]pyrido[3,4-b]pyrazin-5-yl]oxy]methyl]-4-morpholinyl]-ethanone; 1-[(2S)-2-[[[7-[4-[1-(ethylsulfonyl)-4-piperidinyl]phenyl]pyrido[3,4-b]pyrazin-5-yl]oxy]methyl]-4-morpholinyl]-ethanone; α,α-dimethyl-1-[4-[5-[[(2S)-4-(methylsulfonyl)-2-morpholinyl]methoxy]pyrido[3,4-b]pyrazin-7-yl]phenyl]-4-piperidinemethanol; 5-[[(2S)-4-(methylsulfonyl)-2-morpholinyl]methoxy]-7-[4-(1-piperidinyl)phenyl]-pyrido[3,4-b]pyrazine; 7-[4-(4,4-difluoro-1-piperidinyl)phenyl]-5-[[(2S)-4-(methylsulfonyl)-2-morpholinyl]methoxy]-pyrido[3,4-b]pyrazine; 7-[4-[(2R,6S)-2,6-dimethyl-4-morpholinyl]phenyl]-5-[[(2S)-4-(methylsulfonyl)-2-morpholinyl]methoxy]-pyrido[3,4-b]pyrazine; 7-[4-(2-methyl-4-morpholinyl)phenyl]-5-[[(2S)-4-(methylsulfonyl)-2-morpholinyl]methoxy]-pyrido[3,4-b]pyrazine; 1-[(2S)-2-[[[7-[4-(4-ethyl-1-piperazinyl)phenyl]pyrido[3,4-b]pyrazin-5-yl]oxy]methyl]-4-morpholinyl]-2,2-difluoro-ethanone; 2,2-difluoro-1-[(2S)-2-[[[7-[4-(4-methyl-1-piperazinyl)phenyl]pyrido[3,4-b]pyrazin-5-yl]oxy]methyl]-4-morpholinyl]-ethanone; 2,2-difluoro-1-[(2S)-2-[[[7-[3-fluoro-4-(4-morpholinyl)phenyl]pyrido[3,4-b]pyrazin-5-yl]oxy]methyl]-4-morpholinyl]-ethanone; 5-[[(2S)-4-(methylsulfonyl)-2-morpholinyl]methoxy]-7-[4-(1-piperazinyl)phenyl]-pyrido[3,4-b]pyrazine; 7-[4-(4-methyl-1-piperazinyl)phenyl]-5-[[(2S)-4-(methylsulfonyl)-2-morpholinyl]methoxy]-pyrido[3,4-b]pyrazine; 5-[[(2S)-4-(methylsulfonyl)-2-morpholinyl]methoxy]-7-[4-(4-morpholinyl)phenyl]-pyrido[3,4-b]pyrazine; 5-[[(2S)-4-(methylsulfonyl)-2-morpholinyl]methoxy]-7-[4-(tetrahydro-2H-pyran-4-yl)phenyl]-pyrido[3,4-b]pyrazine; 5-[[(2S)-4-(methylsulfonyl)-2-morpholinyl]methoxy]-7-[4-(4-piperidinyl)phenyl]-pyrido[3,4-b]pyrazine; 2,2-difluoro-1-[(2S)-2-[[[7-[4-(tetrahydro-2H-pyran-4-yl)phenyl]pyrido[3,4-b]pyrazin-5-yl]oxy]methyl]-4-morpholinyl]-ethanone; 7-[4-(1-methyl-4-piperidinyl)phenyl]-5-[[(2S)-4-(methylsulfonyl)-2-morpholinyl]methoxy]-pyrido[3,4-b]pyrazine; 7-[3-fluoro-4-(4-morpholinyl)phenyl]-5-[[(2S)-4-(methylsulfonyl)-2-morpholinyl]methoxy]-pyrido[3,4-b]pyrazine; 2,2-difluoro-1-[(2S)-2-[[[7-[4-(1-piperazinyl)phenyl]pyrido[3,4-b]pyrazin-5-yl]oxy]methyl]-4-morpholinyl]-ethanone; 2-[[[7-[4-[1-(methylsulfonyl)-4-piperidinyl]phenyl]pyrido[3,4-b]pyrazin-5-yl]oxy]methyl]-3-morpholinone; 2-[[[7-[4-(1-hydroxy-1-methylethyl)phenyl]pyrido[3,4-b]pyrazin-5-yl]oxy]methyl]-3-morpholinone; 2-[[[7-[4-(1-hydroxy-1-methylethyl)phenyl]pyrido[3,4-b]pyrazin-5-yl]oxy]methyl]-4-methyl-3-morpholinone; 4-methyl-2-[[[7-[4-[1-(methylsulfonyl)-4-piperidinyl]phenyl]pyrido[3,4-b]pyrazin-5-yl]oxy]methyl]-3-morpholinone; 5-[[[7-[4-(4-methyl-1-piperazinyl)phenyl]pyrido[3,4-b]pyrazin-5-yl]oxy]methyl]-2-piperidinone; 5-[[[7-[4-(4-morpholinyl)phenyl]pyrido[3,4-b]pyrazin-5-yl]oxy]methyl]-2-piperidinone; 4-[[7-[4-(4-morpholinyl)phenyl]pyrido[3,4-b]pyrazin-5-yl]oxy]-benzamide; 7-[4-[(2R,6S)-2,6-dimethyl-4-morpholinyl]phenyl]-5-[[(3R)-tetrahydro-3-furanyl]oxy]-pyrido[3,4-b]pyrazine; α,α-dimethyl-1-[4-[5-[[(3R)-tetrahydro-3-furanyl]oxy]pyrido[3,4-b]pyrazin-7-yl]phenyl]-4-piperidinemethanol; 4-[4-[5-[[(3R)-tetrahydro-3-furanyl]oxy]pyrido[3,4-b]pyrazin-7-yl]phenyl]-1-piperidineethanol; 5-[[(3R)-tetrahydro-3-furanyl]oxy]-7-[4-(tetrahydro-2H-pyran-4-yl)phenyl]-pyrido[3,4-b]pyrazine; 7-[3-methyl-4-[4-(methylsulfonyl)-1-piperazinyl]phenyl]-5-[[(3R)-tetrahydro-3-furanyl]oxy]-pyrido[3,4-b]pyrazine; 1-[4-[2-methyl-4-[5-[[(3R)-tetrahydro-3-furanyl]oxy]pyrido[3,4-b]pyrazin-7-yl]phenyl]-1-piperazinyl]-ethanone; 7-[3-chloro-4-(4-morpholinyl)phenyl]-5-[[(3R)-tetrahydro-3-furanyl]oxy]-pyrido[3,4-b]pyrazine; 7-[3-fluoro-4-(4-morpholinyl)phenyl]-5-[[(3R)-tetrahydro-3-furanyl]oxy]-pyrido[3,4-b]pyrazine; 7-[3-methyl-4-(4-morpholinyl)phenyl]-5-[[(3R)-tetrahydro-3-furanyl]oxy]-pyrido[3,4-b]pyrazine; 1-[4-[4-[5-[[(3R)-tetrahydro-2H-pyran-3-yl]oxy]pyrido[3,4-b]pyrazin-7-yl]phenyl]-1-piperidinyl]-ethanone; 7-[4-(4-piperidinyl)phenyl]-5-[[(3R)-tetrahydro-3-furanyl]oxy]-pyrido[3,4-b]pyrazine; 7-[4-[1-(methylsulfonyl)-4-piperidinyl]phenyl]-5-[[(3R)-tetrahydro-2H-pyran-3-yl]oxy]-pyrido[3,4-b]pyrazine; 7-[4-[4-(methylsulfonyl)-1-piperazinyl]phenyl]-5-[[(3R)-tetrahydro-2H-pyran-3-yl]oxy]-pyrido[3,4-b]pyrazine; 1-[4-[4-[5-[[(3R)-tetrahydro-2H-pyran-3-yl]oxy]pyrido[3,4-b]pyrazin-7-yl]phenyl]-1-piperazinyl]-ethanone; 7-[4-[1-(methylsulfonyl)-4-piperidinyl]phenyl]-5-[(tetrahydro-2-furanyl)methoxy]-pyrido[3,4-b]pyrazine; 7-[4-[1-(methylsulfonyl)-4-piperidinyl]phenyl]-5-[(tetrahydro-2H-pyran-2-yl)methoxy]-pyrido[3,4-b]pyrazine; N,N-dimethyl-4-[5-[(tetrahydro-2-furanyl)methoxy]pyrido[3,4-b]pyrazin-7-yl]-benzenamine; N,N-dimethyl-4-[5-[(tetrahydro-2H-pyran-2-yl)methoxy]pyrido[3,4-b]pyrazin-7-yl]-benzenamine; 1-[4-[4-[5-[[(3R)-tetrahydro-3-furanyl]oxy]pyrido[3,4-b]pyrazin-7-yl]phenyl]-1-piperidinyl]-ethanone; N,N,2-trimethyl-4-[5-[[(3R)-tetrahydro-3-furanyl]oxy]pyrido[3,4-b]pyrazin-7-yl]-benzenamine; 2-chloro-N,N-dimethyl-4-[5-[[(3R)-tetrahydro-3-furanyl]oxy]pyrido[3,4-b]pyrazin-7-yl]-benzenamine; 7-[4-[4-(methylsulfonyl)-1-piperazinyl]phenyl]-5-[[(3R)-tetrahydro-3-furanyl]oxy]-pyrido[3,4-b]pyrazine; 1-[4-[4-[5-[[(3R)-tetrahydro-3-furanyl]oxy]pyrido[3,4-b]pyrazin-7-yl]phenyl]-1-piperazinyl]-ethanone; N,N-diethyl-4-[5-[[(3R)-tetrahydro-3-furanyl]oxy]pyrido[3,4-b]pyrazin-7-yl]-benzenamine; 7-(3,4-dimethoxyphenyl)-5-[[(3R)-tetrahydro-3-furanyl]oxy]-pyrido[3,4-b]pyrazine; 7-[4-(4-morpholinyl)phenyl]-5-[[(3R)-tetrahydro-3-furanyl]oxy]-pyrido[3,4-b]pyrazine; N,N-dimethyl-4-[5-[(tetrahydro-2H-pyran-3-yl)oxy]pyrido[3,4-b]pyrazin-7-yl]-benzenamine; 7-[4-(4-methyl-1-piperazinyl)phenyl]-5-[[(3R)-tetrahydro-3-furanyl]oxy]-pyrido[3,4-b]pyrazine; 7-[4-[1-(methylsulfonyl)-4-piperidinyl]phenyl]-5-[[(3R)-tetrahydro-3-furanyl]oxy]-pyrido[3,4-b]pyrazine; N,N-dimethyl-4-[5-[[(3R)-tetrahydro-3-furanyl]oxy]pyrido[3,4-b]pyrazin-7-yl]-benzenamine; N,N-dimethyl-4-[5-[(tetrahydro-2H-pyran-4-yl)oxy]pyrido[3,4-b]pyrazin-7-yl]-benzenamine; N,N-dimethyl-4-[5-[[(3 S)-tetrahydro-3-furanyl]oxy]pyrido[3,4-b]pyrazin-7-yl]-benzenamine; 7-[4-(4-morpholinyl)phenyl]-5-[2-(1H-pyrazol-4-yl)ethoxy]-pyrido[3,4-b]pyrazine; 4-[[7-[4-(4-morpholinyl)phenyl]pyrido[3,4-b]pyrazin-5-yl]oxy]-cyclohexanol.

Benzamide and nicotinamde SYKi compounds are disclosed in U.S. Patent Publication No. 2015/0038492, the content of which are incorporated by reference herein.

The SYKi compounds disclosed in the following patents or patent publications can be utilized: U.S. Pat. Nos. 9,416,111; 9,334,278; 8,299,056; and U.S. Pat. Nos. 9,290,490; 8,470,835; and U.S. Patent Publication Nos. 2016/0185744; 2016/0130659; 2016/0058758; 2016/0045508; 2016/0052930; 2016/0031894; and 2016/0002221.

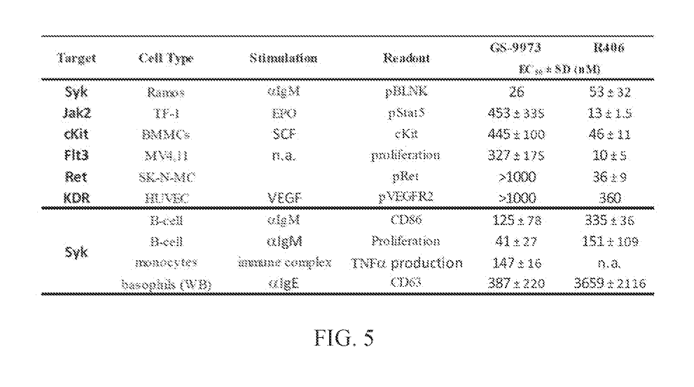

In certain embodiments, the SYKi has selective activity against spleen tyrosine kinase in comparison with the activity of the compound against one more kinases. In certain embodiments, the SYKi is at least ten fold more active against SYK compared to one or more kinases selected from Jak2, cKit, Flt3, Ret and KDR. In certain embodiments, the SYKi is at least twenty fold more active against SYK compared to one or more kinases selected from Jak2, cKit, Flt3, Ret and KDR. For example, as shown in FIG. 5, GS-9973 is at least ten fold more active against SYK than protein kinases Jak2, cKit, Flt3, Ret and KDR.

### Pharmaceutical Compositions and Modes of Administration

Compounds provided herein are usually administered in the form of pharmaceutical compositions. Thus, provides herein are also pharmaceutical compositions that contain one or more of the compounds of any of the formulae disclosed herein or a pharmaceutically acceptable salt, isomers, prodrug, or solvate thereof, and one or more pharmaceutically acceptable vehicles selected from carriers, adjuvants and excipients. Suitable pharmaceutically acceptable vehicles may include, for example, inert solid diluents and fillers, diluents, including sterile aqueous solution and various organic solvents, permeation enhancers, solubilizers and adjuvants. Such compositions are prepared in a manner well known in the pharmaceutical art. See, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

The pharmaceutical compositions may be administered in either single or multiple doses. The pharmaceutical composition may be administered by various methods including, for example, rectal, buccal, intranasal and transdermal routes. In certain embodiments, the pharmaceutical composition may be administered by intra-arterial injection, intravenously, intraperitoneally, parenterally, intramuscularly, subcutaneously, orally, topically, or as an inhalant. In some embodiments, the pharmaceutical composition is administered orally.

One mode for administration is parenteral, for example, by injection. The forms in which the pharmaceutical compositions described herein may be incorporated for administration by injection include, for example, aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Oral administration may be another route for administration of the compounds described herein. Administration may be via, for example, capsule or enteric coated tablets. In making the pharmaceutical compositions that include at least one compound of any of the formulae described herein or a pharmaceutically acceptable salt, prodrug, or solvate thereof, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders. In certain embodiments, the pharmaceutical composition is in the form of tablets.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include lubricating agents such as talc, magnesium stearate, and mineral oil; wetting agents; emulsifying and suspending agents; preserving agents such as methyl and propylhydroxy-benzoates; sweetening agents; and flavoring agents.

The compositions that include at least one compound of any of the formulae described herein or a pharmaceutically acceptable salt, isomer, prodrug, or solvate thereof, can be formulated so as to provide quick, sustained or delayed release of the active ingredient after administration to the subject by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present application employs transdermal delivery devices (“patches”). Such transdermal patches may be used to provide continuous or discontinuous infusion of the compounds described herein in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

For preparing solid compositions such as tablets, the principal active ingredient may be mixed with a pharmaceutical excipient to form a solid preformulation composition containing a homogeneous mixture of a compound of any of the above formulae or a pharmaceutically acceptable salt, prodrug, or solvate thereof. When referring to these preformulation compositions as homogeneous, the active ingredient may be dispersed evenly throughout the composition so that the composition may be readily subdivided into equally effective unit dosage forms such as tablets, pills and capsules.

The tablets or pills of the compounds described herein may be coated or otherwise compounded to provide a dosage form affording the advantage of prolonged action, or to protect from the acid conditions of the stomach. For example, the tablet or pill can include an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation may include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. In other embodiments, compositions in pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

### Dosing

The specific dose level of a compounds described herein for any particular subject will depend upon a variety of factors including the activity of the specific compound employed, the age, body weight, general health, sex, diet, time of administration, route of administration, and rate of excretion, drug combination and the severity of the particular disease in the subject undergoing therapy. For example, a dosage may be expressed as a number of milligrams of a compound of the formula per kilogram of the subject's body weight (mg/kg). Dosages of between about 0.01 and 200 mg/kg may be appropriate. In some embodiments, about 0.01 and 150 mg/kg may be appropriate. In other embodiments a dosage of between 0.05 and 100 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a compound of the formulae administered per dose or per day. Daily dosage of a compound may be between about 1 mg and 2,000 mg, between about 1,000 to 2,000 mg/day, between about 1 to 1,000 mg/day, between about 1 to 500 mg/day, between about 100 to 150 mg/day, between about 1 to 100 mg/day, between about between about 1 to 50 mg/day, between about 50 to 100 mg/day, between about 100 to 125 mg/day, between about 100 to 150 mg/day, between about 100 to 175 mg/day, between about 100 to 200 mg/day, between about 100 to 225 mg/day, between about 100 to 250 mg/day, between about 100 to 350 mg/day, between about 100 to 400 mg/day, between about 100 to 450 mg/day, or between about 100 to 500 mg/day.

When administered orally, the total daily dosage for a human subject may be between 1 mg and 1,000 mg/day, between about 1 to 100 mg/day, between about 1 to 50 mg/day, between about 50 to 100 mg/day, between 50 to 300 mg/day, between 50 to 200 mg/day, between 75 to 200 mg/day, between 75 to 150 mg/day, between 100 to 200 mg/day, between about 200 to 300 mg/day, between about 300 to 400 mg/day, between about 400 to 500 mg/day, between about 100 to 150 mg/day, between about 150 to 200 mg/day, between about 200 to 250 mg/day, between about 75 to 150 mg/day, or between about 150 to 300 mg/day.

The compounds of the present application or the compositions thereof may be administered once, twice, three, or four times daily, using any suitable mode described above.

### Anti-Cancer Treatments

In some embodiments, the application provides methods for treating a patient that is undergoing at least one, at least two, at least three, or at least four anti-cancer therapy (including, for example, standard or experimental chemotherapy) selected from fludarabine, rituximab, obinutuzumab, alkylating agents, alemtuzumab and other chemotherapy treatments such as CHOP (cyclophosphamide, doxorubicin, vincristine, prednisone); R-CHOP (rituximab-CHOP); hyperCVAD (hyperfractionated cyclophosphamide, vincristine, doxorubicin, dexamethasone, methotrexate, cytarabine); R-hyperCVAD (rituximab-hyperCVAD); FCM (fludarabine, cyclophosphamide, mitoxantrone); R-FCM (rituximab, fludarabine, cyclophosphamide, mitoxantrone); bortezomib and rituximab; temsirolimus and rituximab; temsirolimus and Velcade®; Iodine-131 tositumomab (Bexxar®) and CHOP; CVP (cyclophosphamide, vincristine, prednisone); R-CVP (rituximab-CVP); ICE (iphosphamide, carboplatin, etoposide); R-ICE (rituximab-ICE); FCR (fludarabine, cyclophosphamide, rituximab); FR (fludarabine, rituximab); and D.T. PACE (dexamethasone, thalidomide, cisplatin, Adriamycin®, cyclophosphamide, etoposide). Other examples of chemotherapy treatments (including standard or experimental chemotherapies) are described below. In addition, treatment of certain lymphomas is reviewed in Cheson, B. D., Leonard, J. P., “Monoclonal Antibody Therapy for B-Cell Non-Hodgkin's Lymphoma” The New England Journal of Medicine 2008, 359(6), p. 613-626; and Wierda, W. G., “Current and Investigational Therapies for Patients with CLL” Hematology 2006, p. 285-294. Lymphoma incidence patterns in the United States is profiled in Morton, L. M., et al. “Lymphoma Incidence Patterns by WHO Subtype in the United States, 1992-2001” Blood 2006, 107(1), p. 265-276. In some embodiments, the patient is refractory to at least one, at least two, at least three, or at least four of the above anti-cancer therapy.

In some embodiments, the patient is undergoing treatment of non-Hodgkin's lymphomas (NHL), especially of B-cell origin and the treatment includes the use of monoclonal antibodies, standard chemotherapy approaches (e.g., CHOP, CVP, FCM, MCP, and the like), radioimmunotherapy, and combinations thereof, especially integration of an antibody therapy with chemotherapy. Examples of unconjugated monoclonal antibodies for Non-Hodgkin's lymphoma/B-cell cancers include rituximab, alemtuzumab, human or humanized anti-CD20 antibodies, lumiliximab, anti-TRAIL, bevacizumab, galiximab, epratuzumab, SGN-40, and anti-CD74. Examples of experimental antibody agents used in treatment of Non-Hodgkin's lymphoma/B-cell cancers include ofatumumab, ha20, PRO131921, alemtuzumab, galiximab, SGN-40, CHIR-12.12, epratuzumab, lumiliximab, apolizumab, milatuzumab, and bevacizumab. Examples of standard regimens of chemotherapy for Non-Hodgkin's lymphoma/B-cell cancers include CHOP (cyclophosphamide, doxorubicin, vincristine, prednisone), FCM (fludarabine, cyclophosphamide, mitoxantrone), CVP (cyclophosphamide, vincristine and prednisone), MCP (mitoxantrone, chlorambucil, and prednisolone), R-CHOP (rituximab plus CHOP), R-FCM (rituximab plus FCM), R-CVP (rituximab plus CVP), and R-MCP (R-MCP). Examples of radioimmunotherapy for Non-Hodgkin's lymphoma/B-cell cancers include yttrium-90-labeled ibritumomab tiuxetan, and iodine-131-labeled tositumomab.

In another example, the patient is undergoing therapeutic treatments for mantle cell lymphoma (MCL) including combination chemotherapies such as CHOP (cyclophosphamide, doxorubicin, vincristine, prednisone), hyperCVAD (hyperfractionated cyclophosphamide, vincristine, doxorubicin, dexamethasone, methotrexate, cytarabine) and FCM (fludarabine, cyclophosphamide, mitoxantrone). In addition, these regimens can be supplemented with the monoclonal antibody rituximab (Rituxan) to form combination therapies R-CHOP, hyperCVAD-R, and R-FCM. Other approaches include combining any of the abovementioned therapies with stem cell transplantation or treatment with ICE (iphosphamide, carboplatin and etoposide). Other approaches to treating mantle cell lymphoma includes immunotherapy such as using monoclonal antibodies like Rituximab (Rituxan). Rituximab can be used for treating indolent B-cell cancers, including marginal-zone lymphoma, WM, CLL and small lymphocytic lymphoma. A modified approach is radioimmunotherapy, wherein a monoclonal antibody is combined with a radioisotope particle, such as Iodine-131 tositumomab (Bexxar®) and Yttrium-90 ibritumomab tiuxetan (Zevalin®). In another example, Bexxar® is used in sequential treatment with CHOP. Another immunotherapy example includes using cancer vaccines, which is based upon the genetic makeup of an individual patient's tumor. A lymphoma vaccine example is GTOP-99 (MyVax®). Yet other approaches to treating mantle cell lymphoma includes autologous stem cell transplantation coupled with high-dose chemotherapy, or treating mantle cell lymphoma includes administering proteasome inhibitors, such as Velcade® (bortezomib or PS-341), or antiangiogenesis agents, such as thalidomide, especially in combination with Rituxan. Another treatment approach is administering drugs that lead to the degradation of Bcl-2 protein and increase cancer cell sensitivity to chemotherapy, such as oblimersen (Genasense) in combination with other chemotherapeutic agents. Another treatment approach includes administering mTOR inhibitors, which can lead to inhibition of cell growth and even cell death; a non-limiting example is Temsirolimus (CCI-779), and Temsirolimus in combination with Rituxan®, Velcade® or other chemotherapeutic agents.

Other recent therapies for MCL have been disclosed (Nature Reviews; Jares, P. 2007). Such examples include Flavopiridol, PD0332991, R-roscovitine (Selicilib, CYC202), Styryl sulphones, Obatoclax (GX15-070), TRAIL, Anti-TRAIL DR4 and DR5 antibodies, Temsirolimus (CCl-779), Everolimus (RAD001), BMS-345541, Curcumin, Vorinostat (SAHA), Thalidomide, lenalidomide (Revlimid®, CC-5013), and Geldanamycin (17-AAG).

Examples of other therapeutic agents used to treat Waldenstrom's Macroglobulinemia (WM) include perifosine, bortezomib (Velcade®), rituximab, sildenafil citrate (Viagra®), CC-5103, thalidomide, epratuzumab (hLL2-anti-CD22 humanized antibody), simvastatin, enzastaurin, campath-1H, dexamethasone, DT PACE, oblimersen, antineoplaston A10, antineoplaston AS2-1, alemtuzumab, beta alethine, cyclophosphamide, doxorubicin hydrochloride, prednisone, vincristine sulfate, fludarabine, filgrastim, melphalan, recombinant interferon alfa, carmustine, cisplatin, cyclophosphamide, cytarabine, etoposide, melphalan, dolastatin 10, indium In 111 monoclonal antibody MN-14, yttrium Y 90 humanized epratuzumab, anti-thymocyte globulin, busulfan, cyclosporine, methotrexate, mycophenolate mofetil, therapeutic allogeneic lymphocytes, Yttrium Y 90 ibritumomab tiuxetan, sirolimus, tacrolimus, carboplatin, thiotepa, paclitaxel, aldesleukin, recombinant interferon alfa, docetaxel, ifosfamide, mesna, recombinant interleukin-12, recombinant interleukin-11, Bcl-2 family protein inhibitor ABT-263, denileukin diftitox, tanespimycin, everolimus, pegfilgrastim, vorinostat, alvocidib, recombinant flt3 ligand, recombinant human thrombopoietin, lymphokine-activated killer cells, amifostine trihydrate, aminocamptothecin, irinotecan hydrochloride, caspofungin acetate, clofarabine, epoetin alfa, nelarabine, pentostatin, sargramostim, vinorelbine ditartrate, WT-1 analog peptide vaccine, WT1 126-134 peptide vaccine, fenretinide, ixabepilone, oxaliplatin, monoclonal antibody CD19, monoclonal antibody CD20, omega-3 fatty acids, mitoxantrone hydrochloride, octreotide acetate, tositumomab and iodine 1-131 tositumomab, motexafin gadolinium, arsenic trioxide, tipifamib, autologous human tumor-derived HSPPC-96, veltuzumab, bryostatin 1, and PEGylated liposomal doxorubicin hydrochloride, and any combination thereof.

Examples of therapeutic procedures used to treat WM include peripheral blood stem cell transplantation, cord stem cell transplantation, autologous hematopoietic stem cell transplantation, autologous bone marrow transplantation, antibody therapy, biological therapy, enzyme inhibitor therapy, total body irradiation, infusion of stem cells, bone marrow ablation with stem cell support, in vitro-treated peripheral blood stem cell transplantation, umbilical cord blood transplantation, immunoenzyme technique, pharmacological study, low-LET cobalt-60 gamma ray therapy, bleomycin, conventional surgery, radiation therapy, and nonmyeloablative allogeneic hematopoietic stem cell transplantation.

Examples of other therapeutic agents used to treat diffuse large B-cell lymphoma (DLBCL) drug therapies (Blood 2005 Abramson, J.) include cyclophosphamide, doxorubicin, vincristine, prednisone, anti-CD20 monoclonal antibodies, etoposide, bleomycin, many of the agents listed for Waldenstrom's, and any combination thereof, such as ICE and R-ICE.

Examples of other therapeutic agents used to treat chronic lymphocytic leukemia (CLL) (Spectrum, 2006, Fernandes, D.) include Chlorambucil (Leukeran), Cyclophosphamide (Cyloxan, Endoxan, Endoxana, Cyclostin), Fludarabine (Fludara), Pentstatin (Nipent), Cladribine (Leustarin), Doxorubicin (Adriamycin®, Adriblastine), Vincristine (Oncovin), Prednisone, Prednisolone, Alemtuzumab (Campath, MabCampath), many of the agents listed for Waldenstrom's, and combination chemotherapy and chemoimmunotherapy, including the common combination regimen: CVP (cyclophosphamide, vincristine, prednisone); R-CVP (rituximab-CVP); ICE (iphosphamide, carboplatin, etoposide); R-ICE (rituximab-ICE); FCR (fludarabine, cyclophosphamide, rituximab); and FR (fludarabine, rituximab).

### Example

In this phase 1b/2 study (NCT02343939), patients age 18 to 70 years with previously untreated AML, preserved organ function, and ECOG≦2 were eligible to receive dose escalated entospletinib for 14 days as monotherapy (days −14 to 0) followed by combination with daunorubicin 60 mg/m2/d, cycle 1 day 1 to 3, and cytarabine 100 mg/m2/d, cycle 1 day 1 to 7. All patients received entospletinib monotherapy for up to 14 days prior to starting induction. Chemotherapy could be initiated after 5 days of monotherapy (and entospletinib continued for 4+ weeks) in patients with leukemia-related complications necessitating chemotherapy. Patients enrolled to dose level (DL) 0 and DL 1 received entospletinib (also referred to herein as Compound 1, ENTO or GS-9973) 200 mg po BID and 400 mg po BID, respectively. Patients with residual disease two weeks after chemotherapy received a second induction cycle identical to the first. Entospletinib was continued without interruption until remission was assessed at count recovery.

Twelve patients enrolled with a median age of 54 (range, 18-69) years. Patients were in the following European LeukemiaNet genetic risk groups: favorable (n=1), intermediate I (n=3), intermediate II (n=2), and adverse (n=4), respectively. Three patients were not evaluable for dose limiting toxicity (DLT) assessment and were replaced (due to detection of CNS disease requiring non-study therapy (n=1), and withdrawal of consent unrelated to drug toxicity (n=2)). Single-agent entospletinib during the window period was well tolerated; toxicities after combination with intensive chemotherapy were common and typical. Among three patients treated at 200 mg BID, no DLT was observed. Of three patients treated at 400 mg BID, a patient with documented fungal pneumonia developed grade 3 pneumonitis that was possibly related to entospletinib. Although this did not meet DLT criteria, DL 1 was expanded with 3 additional patients, none of whom experienced DLT. Overall, the most common non hematologic adverse events (inclusive of intensive chemotherapy periods) were febrile neutropenia, nausea, and diarrhea. Based on this clinical experience and compiled pharmacokinetic data demonstrating lack of benefit to further dose escalation, 400 mg BID was selected as the recommended phase 2 dose. Responses were seen at both levels. Among the 3 patients treated at 200 mg BID, two required a second induction but each achieved a complete remission (CR) (3/3; 100%). Of the 6 patients treated at 400 mg BID, none required a second induction and the CR rate was also 100%. Remarkably, an 18 year old male with 11q23-rearranged AML achieved morphologic and cytogenetic CR after only the 14 day entospletinib monotherapy window (prior to chemotherapy). Another patient with 11q23-rearranged AML had significant platelet response during the window period (this patient refused disease evaluation by marrow aspiration prior to chemotherapy).

Entospletinib appears to have significant clinical activity in AML, and its combination at doses up to 400 mg BID with intensive chemotherapy is well tolerated. Patients with 11q23-rearranged AML can be seen as sensitive to SYK inhibition by entospletinib.

Throughout this specification, various patents, patent applications and other types of publications (e.g., journal articles) are referenced. The disclosure of all patents, patent applications, and publications cited herein are hereby incorporated by reference in their entirety for all purposes.

What is claimed is:

1. A method of treating a reducing tumor burden or leukemic burden in a patient in need thereof, comprising the step of administering an effective amount of an inhibitor of spleen tyrosine kinase (SYKi).

2. A method of increasing the platelet count in a patient receiving chemotherapy or radiotherapy comprising the step of administering to said patient an effective amount of of an inhibitor of spleen tyrosine kinase (SYKi).

3. A method of increasing the neutrophil count in a patient receiving chemotherapy or radiotherapy comprising the step of administering to said patient an effective amount of an inhibitor of spleen tyrosine kinase (SYKi).

4. A method of increasing bone marrow production, neutrophil count or platelet count in a patient diagnosed with myelodysplastic syndrome (MDS) comprising the step of administering an effective amount of an inhibitor of spleen tyrosine kinase (SYKi).

5. A method of decreasing myelosuppression, comprising administering an effective amount of an inhibitor of an inhibitor of spleen tyrosine kinase (SYKi) to a patient in need thereof.

6. The method according to claim 5, wherein said myelosuppression is induced by administration of a myelosuppressive agent to said patient.

7. The method according to claim 6, wherein said myelosuppressive agent is an anti-cancer drug, or a combination of anti-cancer drugs.

8. The method according to any of claims 2-4 and 7, wherein said chemotherapy is an anti-cancer agent selected from a DNA damaging agent, an antibiotic agent, an antimitotic agent, a steroid and a glucocorticoid, or a combination thereof.

9. The method according to claim 8, wherein said anti-cancer drug is selected from a DNA damaging agent, an antibiotic agent, an antimitotic agent, a steroid and a glucocorticoid, or a combination thereof.

10. The method according to claim 9, wherein said DNA alkylating agent is selected from actinomycin, amsacrine, busulfan, carboplatin, chlorambucil, cisplatin, cyclophosphamide, Cytoxan, dactinomycin, daunorubicin, doxorubicin, epirubicin, iphosphamide, melphalan, merchlorehtamine, mitomycin, mitoxantrone, nitrosourea, procarbazine, taxol, taxotere, teniposide, etoposide and triethylenethiophosphoramide.

11. The method according to claim 9, wherein said antibiotic is selected from dactinomycin (actinomycin D), daunorubicin, doxorubicin (adriamycin), idarubicin, anthracyclines, mitoxantrone, bleomycins, plicamycin (mithramycin) and mitomycin.

12. The method according to claim 9, wherein said antimitotic agent is selected from a vinca alkaloid and a taxane, nocodazole, epothilones, navelbine and epidipodophyllotoxins.

13. The method according to claim 12, wherein said vinca alkaloid is selected from vinblastine and vincristine.

14. A method of treating a patient diagnosed with cancer or myelodysplastic syndromes, comprising the step of administering an effective amount of an inhibitor of spleen tyrosine kinase (SYKi) to said patient, one day, two days, three days, four days, five days, six days, a week, two weeks, three weeks, a month or more than a month prior to the initiation of chemotherapy or radiotherapy as pre-treatment.

15. The method according to any of the above claims wherein said patient is diagnosed with a disease or disorder is selected from acute lymphocytic leukemia (ALL), acute myeloid leukemia (AML), chronic lymphocytic leukemia (CLL), small lymphocytic lymphoma (SLL), myelodysplastic syndrome (MDS), myeloproliferative disease (MPD), chronic myeloid leukemia (CML), multiple myeloma (MM), non-Hodgkin's lymphoma (NHL), mantle cell lymphoma (MCL), follicular lymphoma, Waldestrom's macroglobulinemia (WM), T-cell lymphoma, B-cell lymphoma, diffuse large B-cell lymphoma (DLBCL), pancreatic cancer, bladder cancer, colorectal cancer, breast cancer, prostate cancer, renal cancer, hepatocellular cancer, lung cancer, ovarian cancer, cervical cancer, gastric cancer, esophageal cancer, head and neck cancer, melanoma, neuroendocrine cancer, CNS cancer, brain cancer, bone cancer, soft tissue sarcoma, non-small cell lung cancer, small-cell lung cancer and colon cancer.

16. The method according to claim 15, wherein said disease or disorder is acute lymphocytic leukemia (ALL).

17. The method according to claim 15, wherein said disease or disorder is acute myeloid leukemia (AML).

18. The method according to claim 15, wherein said disease or disorder is chronic lymphocytic leukemia (CLL).

19. The method according to claim 15, wherein said disease or disorder is myeloproliferative disease (MPD).

20. The method according to claim 15, wherein said disease or disorder is chronic myeloid leukemia (CML).

21. The method according to claim 15, wherein said disease or disorder is multiple myeloma (MM).

22. The method according to claim 15, wherein said disease or disorder is non-Hodgkin's lymphoma (NHL).

23. The method according to claim 15, wherein said disease or disorder is mantle cell lymphoma (MCL).

24. The method according to claim 15, wherein said disease or disorder is B-cell lymphoma, diffuse large B-cell lymphoma (DLBCL).

25. The method according to any of the above claims wherein said SYKi is Compound 1 having the formula: 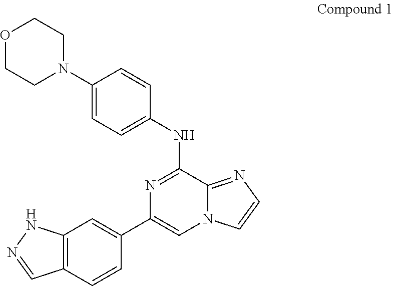 [SMILES: c1cn2cc(-c3ccc4cn[nH]c4c3)nc(Nc3ccc(N4CCOCC4)cc3)c2n1]
or a pharmaceutically acceptable salt, pharmaceutically acceptable co-crystal, pharmaceutically acceptable ester, stereoisomer, mixture of stereoisomers, or tautomer thereof.

26. The method according to any of claims 1-24, wherein said SYKi is a bis-mesylate salt of Compound 1: 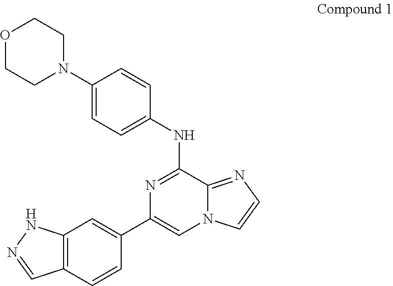 [SMILES: c1cn2cc(-c3ccc4cn[nH]c4c3)nc(Nc3ccc(N4CCOCC4)cc3)c2n1]
or a hydrate thereof.

27. The method according to any of claims 1-24, wherein said SYKi is a compound of formula: 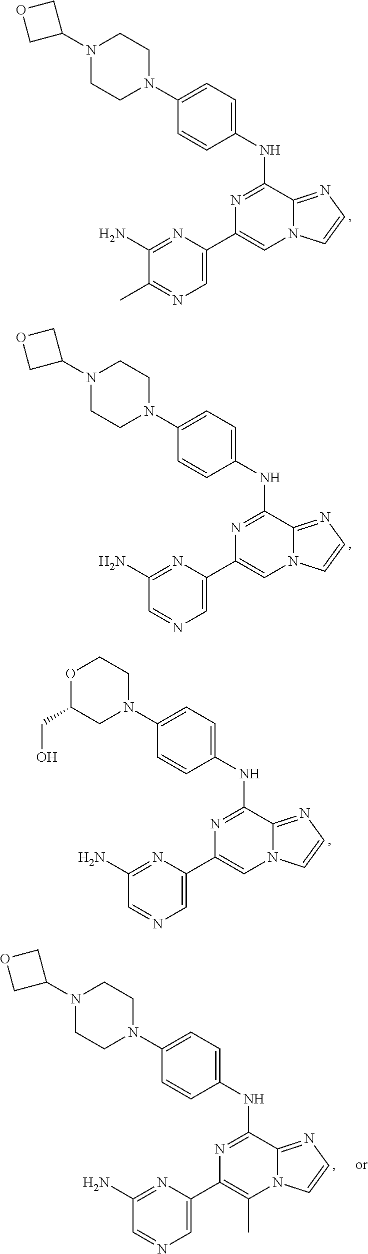 [SMILES: Cc1ncc(-c2cn3ccnc3c(Nc3ccc(N4CCN(C5COC5)CC4)cc3)n2)nc1N ; Nc1cncc(-c2cn3ccnc3c(Nc3ccc(N4CCN(C5COC5)CC4)cc3)n2)n1 ; Nc1cncc(-c2cn3ccnc3c(Nc3ccc(N4CCO[C@@H](CO)C4)cc3)n2)n1 ; Cc1c(-c2cncc(N)n2)nc(Nc2ccc(N3CCN(C4COC4)CC3)cc2)c2nccn12] 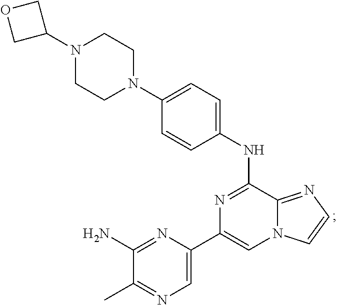 [SMILES: Cc1ncc(-c2cn3ccnc3c(Nc3ccc(N4CCN(C5COC5)CC4)cc3)n2)nc1N]
or a pharmaceutically acceptable salt, pharmaceutically acceptable co-crystal, pharmaceutically acceptable ester, stereoisomer, mixture of stereoisomers, or tautomer thereof.

28. The method according to any of claims 1-24, wherein said SYKi is a compound of formula: 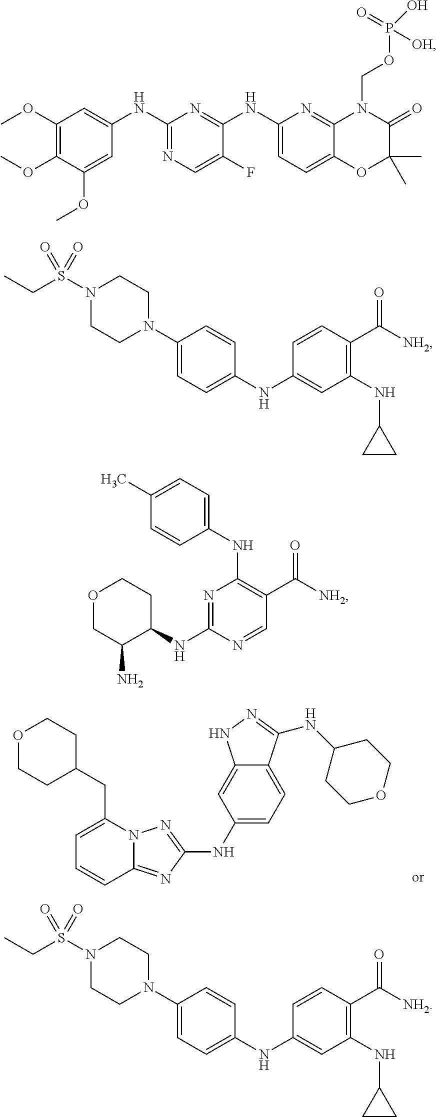
or a pharmaceutically acceptable salt, pharmaceutically acceptable co-crystal, pharmaceutically acceptable ester, stereoisomer, mixture of stereoisomers, or tautomer thereof.

29. The method according to any of claims 5-28, wherein said myelosuppression is selected from neutropenia, pancytopenia, thrombocytopenia, leukopenia and anemia.

30. The method according to any of the above claims wherein said SYKi is administered in combination with one or more additional drugs selected from corticosteroids, glucocorticoids, mineralocorticoids, hydrocortisone, dexamethasone, cortisone, prednisone, prednisolone, methylprednisolone, dexamethasone, betamethasone, triamcinolone, beclometasone, fludrocortisone, fludrocortisone acetate, deoxycorticosterone, deoxycorticosterone acetate, or aldosterone.

31. The method according to claim 30, wherein said additional drug is prednisone.

32. A method for treating AML in a patient with 11q23/MLL abnormalities comprising the step of administering an effective amount of a SYKi to said patient.

33. The method according to claim 32, wherein said SYKi is Compound 1 having the formula: 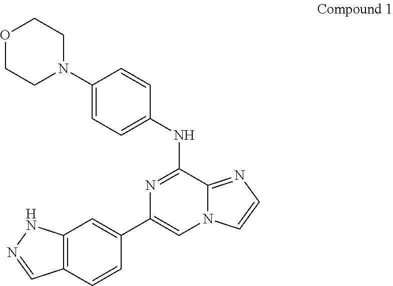 [SMILES: c1cn2cc(-c3ccc4cn[nH]c4c3)nc(Nc3ccc(N4CCOCC4)cc3)c2n1]
or a pharmaceutically acceptable salt, pharmaceutically acceptable co-crystal, pharmaceutically acceptable ester, stereoisomer, mixture of stereoisomers, or tautomer thereof.

34. The method according to claim 32, wherein said SYKi is a bis-mesylate salt of Compound 1: 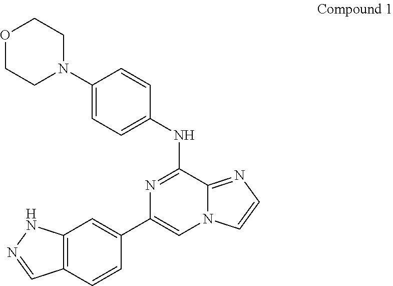 [SMILES: c1cn2cc(-c3ccc4cn[nH]c4c3)nc(Nc3ccc(N4CCOCC4)cc3)c2n1]
or a hydrate thereof.

35. The method according to claim 4, wherein said patient is not undergoing chemotherapy or radiotherapy.

36. The method according to any of the above claims wherein neutrophil count is increased by 10%, 20%, 30%, 40% or 50% after two weeks of treatment with said SYKi.

37. The method according to any of the above claims wherein platelet count is increased by 10%, 20%, 30%, 40% or 50% after two weeks of treatment with said SYKi.

38. The method according to any of claims 7-35, wherein said administration of anti-cancer agent reduces neutrophil or platelet count by 10%, 20%, 30%, 40%, 50%, 60%, 70%, 80% or 90% following one, two, three or four weeks after said administration of anti-cancer drug.

39. A method for protecting and stimulating proliferation of transplanted cells in the bone marrow of a human patient of a bone marrow transplant, wherein prior to the bone marrow transplant, the patient has received a myelosuppressing amount of an anti-cancer drug, said process comprising administering to the patient an effective amount of a SYKi.

40. The method according to claim 39, wherein said SYKi is Compound 1 having the formula: 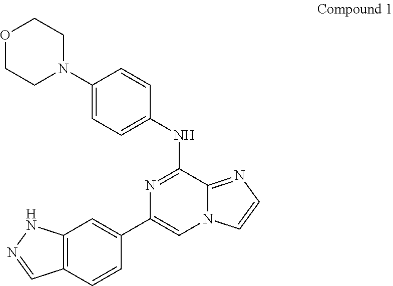 [SMILES: c1cn2cc(-c3ccc4cn[nH]c4c3)nc(Nc3ccc(N4CCOCC4)cc3)c2n1]
or a pharmaceutically acceptable salt, pharmaceutically acceptable co-crystal, pharmaceutically acceptable ester, stereoisomer, mixture of stereoisomers, or tautomer thereof.

41. The method according to claim 39, wherein said SYKi is a bis-mesylate salt of Compound 1: 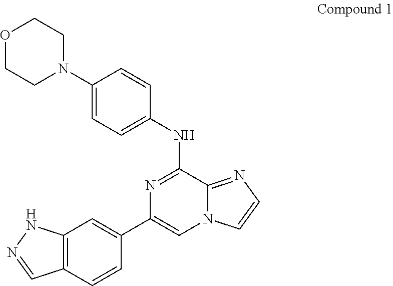 [SMILES: c1cn2cc(-c3ccc4cn[nH]c4c3)nc(Nc3ccc(N4CCOCC4)cc3)c2n1]
or a hydrate thereof.

42. The method according to claim 39, wherein said SYKi is a compound of formula: 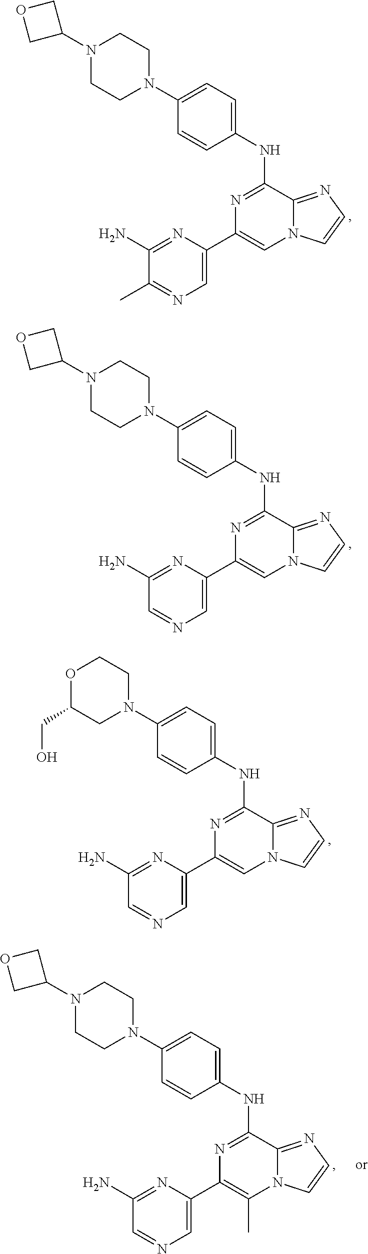 [SMILES: Cc1ncc(-c2cn3ccnc3c(Nc3ccc(N4CCN(C5COC5)CC4)cc3)n2)nc1N ; Nc1cncc(-c2cn3ccnc3c(Nc3ccc(N4CCN(C5COC5)CC4)cc3)n2)n1 ; Nc1cncc(-c2cn3ccnc3c(Nc3ccc(N4CCO[C@@H](CO)C4)cc3)n2)n1 ; Cc1c(-c2cncc(N)n2)nc(Nc2ccc(N3CCN(C4COC4)CC3)cc2)c2nccn12] 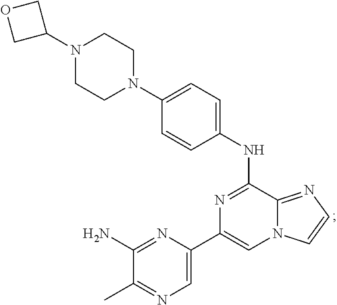 [SMILES: Cc1ncc(-c2cn3ccnc3c(Nc3ccc(N4CCN(C5COC5)CC4)cc3)n2)nc1N]
or a pharmaceutically acceptable salt, pharmaceutically acceptable co-crystal, pharmaceutically acceptable ester, stereoisomer, mixture of stereoisomers, or tautomer thereof.

43. The method according to claim 39, wherein said SYKi is a compound of formula: 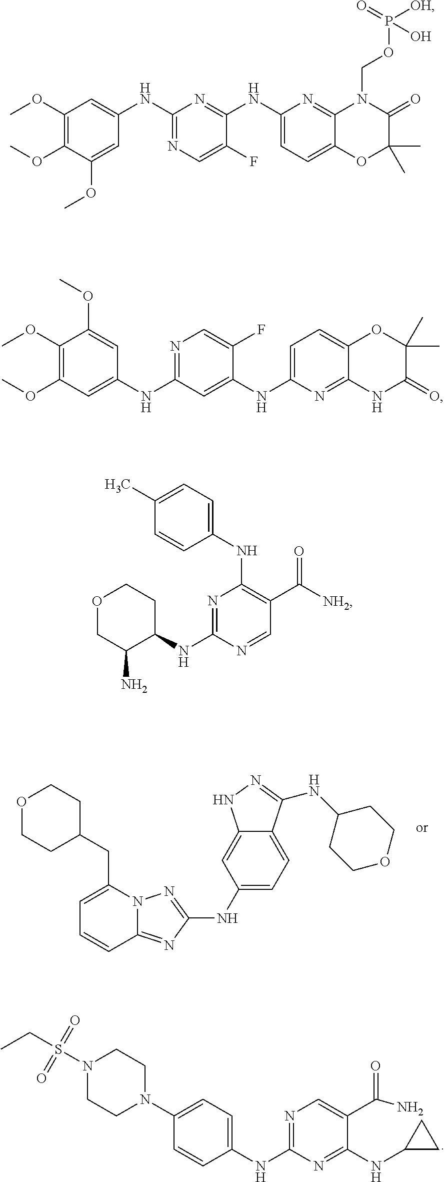
or a pharmaceutically acceptable salt, pharmaceutically acceptable co-crystal, pharmaceutically acceptable ester, stereoisomer, mixture of stereoisomers, or tautomer thereof.

44. A method of treating a patient undergoing radiotherapy comprising the step of administering an effective amount of an inhibitor of spleen tyrosine kinase (SYKi) wherein said SYKi is a radioprotectant.

45. A method of treating a myelosupprssive disorder in a patient diagnosed with or receiving treatment for a solid tumor selected from prostate cancer, pancreatic cancer, bladder cancer, colorectal cancer, breast cancer, renal cancer, hepatocellular cancer, lung cancer, ovarian cancer, cervical cancer, rectum cancer, liver cancer, kidney cancer, stomach cancer, skin cancer, gastric cancer, esophageal cancer, head and neck cancer, melanoma, neuroendocrine cancers, CNS cancers, brain tumors, bone cancer, soft tissue sarcoma, non-small cell lung cancer, small-cell lung cancer, colon cancer or melanoma comprising the step of administering an effective amount of an inhibitor of spleen tyrosine kinase (SYKi).

46. The method according to claim 45, wherein said myleosuppressive disorder is induced by an anti-cancer agent.

47. The method according to claim 46, wherein said anti-cancer agent is selected from enzalutamide, abiraterone, abiraterone acetate, apalutamide, galeterone, olaparib, niraparib, veliparib, rucaparib, flutamide, nilutamide, bicalutamide, ketonazole, orteronel, finasteride, dutasteride, bexlosteride, izonsteride, turosteride, episteride, dexamethasone, prednisone, leuprolide, goserelin, triptorelin, histrelin, estrogen, cyproterone acetate, spironolactone, flutamide, hydroxyflutamide, docetaxel, cabazitaxel, sipuleucel-T, ODM-201, VT-464 and EPI-506, or a combination thereof,

48. The method according to claim 46, wherein said anti-cancer agent is selected from actinomycin, amsacrine, busulfan, carboplatin, chlorambucil, cisplatin, cyclophosphamide, Cytoxan, dactinomycin, daunorubicin, doxorubicin, epirubicin, iphosphamide, melphalan, merchlorehtamine, mitomycin, mitoxantrone, nitrosourea, procarbazine, taxol, taxotere, teniposide, etoposide and triethylenethiophosphoramide, dactinomycin (actinomycin D), daunorubicin, doxorubicin (adriamycin), idarubicin, anthracyclines, mitoxantrone, bleomycins, plicamycin (mithramycin) and mitomycin, vinca alkaloids, taxanes, nocodazole, epothilones, navelbine and epidipodophyllotoxin.
